(19) United States
(12) Patent Application Publication
(10) Pub. No.: US 2024/0139198 A1
(43) Pub. Date: May 2, 2024

(54) METHOD AND COMBINATION FOR TREATING TUMORS

(71) Applicant: CURON BIOPHARMACEUTICAL (SHANGHAI) CO., LIMITED, Shanghai (CN)

(72) Inventors: Rongzhen LU, Shanghai (CN); Zhihong CHEN, Shanghai (CN)

(21) Appl. No.: 18/276,394

(22) PCT Filed: Feb. 9, 2022
(86) PCT No.: PCT/CN2022/075642

§ 371 Date: Aug. 8, 2023

(30) Foreign Application Priority Data
Feb. 10, 2021 (CN) 202110184081.5
Apr. 13, 2021 (CN) 202110410557.2
Jan. 27, 2022 (CN) 202210099148.X

(51) Int. Cl. A61K 31/52; A61K 45/06; A61K 47/68; A61P 35/00; C07K 14/71; C07K 16/22; C07K 16/28

(52) U.S. Cl. CPC A61K 31/52; A61K 45/06; A61K 47/6849; A61P 35/00; C07K 14/71; C07K 16/22; C07K 16/2827

(57) ABSTRACT

The present application relates to a pharmaceutical combination comprising a phosphoinositide 3-kinase (PI3K) inhibitor and a second therapeutic agent, wherein the second therapeutic agent is an immune checkpoint inhibitor, a TGFβ inhibitor, a bifunctional immune checkpoint/TGFβ inhibitors or combinations thereof. The present application also provides a method of treatment comprising administering the combination, and the use of the drug combination for treating tumors.

### FIELD

The present application relates to the field of biomedicine, in particular to a method and combination for treating tumors.

### BACKGROUND

The phosphoinositide 3-kinase (PI3K) signaling pathway is one of the most highly mutated systems in human cancers. PI3Ks are members of a unique and conserved family of intracellular lipid kinases that phosphorylate the 3′-OH group on phosphatidylinositols or phosphoinositides. The PI3K family includes 15 kinases with distinct substrate specificities, expression patterns, and regulatory patterns. Class I PI3Ks (p110α, p110β, p110δ, and p110γ) are normally activated by tyrosine kinases or G-protein-coupled receptors to generate (3,4,5)-phosphatidylinositol triphosphate (PIP3), which engages downstream effectors, such as those in the AKT/PDK1 pathway, mTOR, Tec family kinases and Rho family GTPases. By synthesizing phosphatidylinositol 3-diphosphate (PI(3)P) and (3,4)-phosphatidylinositol diphosphate (PI(3,4)P2), Class II and III PI3Ks plays a key role in intracellularly transport. PI3Ks are protein kinases that control cell growth (mTORCl) or monitor genome integrity (ATM, ATR, DNA-PK, and hSmg-1).

There are four mammalian subtypes of class I PI3Ks: PI3K-alpha, β, delta (class Ia PI3Ks) and PI3K-gamma (class lb PI3Ks). These enzymes catalyze the production of PIP3, leading to the activation of downstream effector pathways important for cell survival, differentiation and function. PI3K-α and PI3K-β are ubiquitously expressed and are important mediators of signal transduction from cell surface receptors. PI3K-α is the most commonly mutated isoform in cancer and plays a role in insulin signaling and glucose homeostasis (Knight et al Cell (2006) 125(4):733-47; Vanhaesebroeck et al Current Topic Microbiol. Immunol. (2010) 347:1-19). PI3K-β is activated in phosphatase and tensin homolog (PTEN)-deficient cancers. Both subtypes are cancer targets for small molecule therapies in development.

PI3K-δ and -γ are preferentially expressed in leukocytes and play a key role in leukocyte function. These subtypes also contribute to the development and maintenance of hematological malignancies (Vanhaesebroeck et al. Current Topic Microbiol. Immunol. (2010) 347:1-19; Clayton et al. J Exp Med. (2002) 196(6):753-19 63; Fung-Leung Cell Signal. (2011) 23(4):603-8; Okkenhaug et al. Science (2002) 297(5583):1031-34). PI3K-delta is activated by cellular receptors (eg, receptor tyrosine kinases) through interaction with the Sarc homology 2 (SH2) domain of the PI3K regulatory subunit (p85) or through direct interaction with RAS.

Transforming growth factor β (TGFβ) is a potent cytokine that has a significant effect on the immune system. The major function of TGFβ in the immune system is to maintain tolerance and initial immune response to foreign pathogens. Three isoforms of TGF[β] have been identified in mammals, TGFβ1, TGFβ2 and TGFβ3, with TGFβ1 being the predominant isoform. TGFβ is secreted in a latent form, and only a small fraction of the total TGFβ secreted is activated under physiological conditions. Most of the biological effects of TGFβ are realized through the binding of TGFβ to receptor ALK5 and TGFβ receptor II (TGFβR2). Specifically, active TGFβ dimers can bind tetrameric ALK5 and TGFβR2 complexes to initiate signal transduction. ALK5 is not required for initial binding of TGFβ but is required for signal transduction.

Programmed death receptor 1 (programmed death 1, PD-1) is a member of the CD28 superfamily PD-1 is expressed in activated T cells, B cells and myeloid cells, and it has two ligands, namely programmed death ligand 1 (PD-L1) and PD-L2. PD-L1 interacts with the receptor PD-1 on T cells and plays an important role in the negative regulation of the immune response. The expression of PD-L1 protein can be detected in many human tumor tissues. The microenvironment of the tumor site can induce the expression of PD-L1 on tumor cells. The expressed PD-L1 is conducive to the occurrence and growth of tumors, and induces anti-tumor Apoptosis of T cells. PD-1/PD-L1 pathway inhibitors can block the combination of PD-1 and PD-L1, block negative regulatory signals, restore the activity of T cells, and enhance the immune response.

Transforming growth factor-β (transforming growth factor-β, TGF-β) belongs to the TGF-β superfamily that regulates cell growth and differentiation. TGF-β deliver signals by a heterotetrameric receptor complex consisting of two type I and two type II transmembrane serine/threonine kinase receptors.

TGFβ can affect many cellular functions, such as cell proliferation, differentiation, cell-cell and cell-matrix adhesion, cell motility, and activation of lymphocytes. (For a review of the role of TGFβ in regulating immune responses, see Li et al. (2006) Annu. Rev. Immunol. 24:99-146.) Furthermore, TGFβ is believed to induce or mediate the progression of many diseases, such as osteoporosis disease, hypertension, atherosclerosis, cirrhosis, fibrotic disease of the kidney, liver, and lung, and tumor progression. In animal models of certain diseases, such as diabetic nephropathy, glomerulonephritis, cyclosporin-mediated kidney injury, and systemic lupus erythematosus (SLE), TGFβ can enhance end-organ damage caused by chronic inflammation, while TGFβ Antagonists can effectively reduce this damage (Border et al. (1990) Nature 346:371-374; Border et al. (1992) Nature 360:361-364; Isaka et al. (1999) Kidneylnt. 55:465-475; Sharma et al. Diabetes 45: 522; Xin et al. (2004) Transplantation 15: 1433; Benigni et al. (2003) J. Am. Soc. Nephrol. 14: 1816). In terms of cancer, TGFβ may have a direct inhibitory effect on malignant cells, and may increase the production or activity of a series of tumor growth factors and angiogenesis factors.

In the treatment of cancer, it has long been recognized that chemotherapy brings high toxicity and can lead to negative effects on drug-resistant cancer cells. Even with therapies that target overexpressed or activated proteins associated with tumor survival and growth, cancer cells mutate to reduce or escape reliance on the pathway targeted by the therapy and exploit other pathways to continue Survive. Tumor immunotherapy mainly improves the immunogenicity of tumor cells and the sensitivity to effector cell killing through immunological principles and methods, stimulates and enhances the body's anti-tumor immune response, and uses immune cells and effector molecules to infuse the host body to cooperate with the body. The immune system kills tumors and inhibits tumor growth. Reducing the toxicity and resistance of tumor drugs, and reducing the side effects are still urgent problems to be solved in tumor treatment.

### SUMMARY

The object of the present application is to provide combinations and methods comprising a PI3K inhibitor and a selected second therapeutic agent. In certain embodiment, a combination of a PI3K inhibitor and a second therapeutic agent selected from one or more of the following has been found to be synergistic in treating tumors (e.g., in reducing cancer cell growth or viability, or both): Inhibitors of immune checkpoints, TGFβ inhibitors, or combinations thereof. The combination of a PI3K inhibitor and a selected second therapeutic agent may allow for a desired lower dose of the PI3K inhibitor, second therapeutic agent, or both, while achieving the same therapeutic effect as compared to monotherapy doses apply. In certain embodiment, the combination can allow for less frequent administration of the PI3K inhibitor, the second therapeutic agent, or both than when the PI3K inhibitor or the second therapeutic agent is administered as a monotherapy. Such a combination may provide an advantageous effect, e.g., in reducing, preventing, delaying and/or reducing the occurrence of one or more of side effects, toxicity or resistance otherwise associated with the administration of higher doses of the agents.

In one aspect, the present application is to provide a pharmaceutical combination comprising a phosphoinositide 3-kinase (PI3K) inhibitor and a second therapeutic agent, wherein the second therapeutic agent is an immune checkpoint inhibitor, a TGFβ inhibitor, a bifunctional immune checkpoint/TGFβ inhibitors or combinations thereof.

In certain embodiment, the immune checkpoint inhibitor comprises an agent capable of blocking the interaction between programmed death 1 (PD-1) and programmed death ligand 1 (PD-L1).

In certain embodiment, the immune checkpoint inhibitor comprises a programmed death ligand 1 (PD-L1) and/or programmed death 1 (PD-1) inhibitor.

In certain embodiment, the bifunctional immune checkpoint/TGFβ inhibitor comprises a PD-L1/TGFβ dual inhibitor, or a PD-1/TGFβ dual inhibitor.

In certain embodiment, the combination comprises:
1) the combination of PD-L1 inhibitor and PI3K inhibitor;
2) the combination of TGFβ inhibitor and PI3K inhibitor;
3) the combination of PD-1 inhibitor, TGFβ inhibitor and PI3K inhibitor;
4) the combination of PD-L1 inhibitor, TGFβ inhibitor and PI3K inhibitor;
5) the combination of PD-L1/TGFβ dual inhibitor and PI3K inhibitor; or
6) the combination of PD-1/TGFβ dual inhibitor and PI3K inhibitor.

In certain embodiment, the immune checkpoint inhibitor comprises a nucleic acid, an antibody or an antigen-binding fragment thereof, a fusion protein or a chemical compound.

In certain embodiment, wherein the inhibitors of immune checkpoints include nucleic acids(e.g. dsRNA, siRNA or sh RNA), polypeptide(e.g. soluble ligands, antibodies or antigen-binding fragments thereof, immuneoadhesins, fusion proteins, oligopeptides and other molecules) or compound.

In certain embodiment, said antibody is selected from the group consisting of: human antibody, humanized antibody, chimeric antibody, multispecific antibody, monoclonal antibody and polyclonal antibody.

In certain embodiment, the antigen-binding fragment is selected from Fab, Fab′, F(ab′)₂, Fv, scFv, bispecific antibody, Fd, dAb, VHH, large antibody and complementarity determining region (CDR) fragments.

In certain embodiment, the antibody or antigen-binding fragment thereof comprises a heavy chain variable region (VH) comprising HCDR1, HCDR2 and HCDR3.

In certain embodiment, the antibody or antigen-binding fragment thereof further comprises or does not comprise a CH1 domain.

In certain embodiment, the antibody or antigen-binding fragment thereof further comprises or does not comprise CH2 and CH3 domains.

In certain embodiment, the antibody or antigen-binding fragment thereof further comprises an antibody heavy chain constant region.

In certain embodiment, the antibody heavy chain constant region is a human antibody heavy chain constant region.

In certain embodiment, the human antibody heavy chain constant region is selected from constant regions derived from the group consisting of IgG1, IgG2, IgG3, IgG4 and variants thereof.

In certain embodiment, the antibody or antigen-binding fragment thereof further comprises a light chain variable region (VL) comprising LCDR1, LCDR2, LCDR3.

In certain embodiment, the antibody or antigen-binding fragment thereof further comprises a light chain constant region (CL).

In certain embodiment, the immune checkpoint inhibitor comprises an anti-PD-L1 antibody or an antigen-binding fragment thereof, or an anti-PD-1 antibody or an antigen-binding fragment thereof.

In certain embodiment, the anti-PD-L1 antibody or antigen-binding fragment thereof comprises a heavy chain variable region of an antibody, wherein the heavy chain variable region comprises HCDR1, HCDR2, HCDR3, which has at least about 80% sequence identity to HCDR1, HCDR2, HCDR3, respectively, of atezolizumab, avelumab, BMS-936559, MPDL3280A (RG7446), or durvalumab (MEDI-4736).

In certain embodiment, the anti-PD-L1 antibody or antigen-binding fragment thereof comprises an antibody heavy chain variable region, wherein the heavy chain variable region is separately associated with The heavy chain variable regions of the following molecules have at least about 80% sequence identity: atezolizumab, avelumab, BMS-936559, MPDL3280A, or durvalumab.

In certain embodiment, the anti-PD-L1 antibody or antigen-binding fragment thereof comprises a heavy chain of an antibody, wherein the heavy chain is respectively associated with a heavy chain of the following molecule having at least about 80% sequence identity: Atezolizumab, Avelumab, BMS-936559, MPDL3280A, or durvalumab.

In certain embodiment, the anti-PD-L1 antibody or antigen-binding fragment thereof further comprises an antibody light chain variable region, wherein the light chain variable region comprises LCDR1, LCDR2, LCDR3, each of which has at least about 80% sequence identity to LCDR1, LCDR2, LCDR3, respectively, of atezolizumab, avelumab, BMS-936559, MPDL3280A, or durvalumab.

For example, the anti-PD-L1 antibody or antigen-binding fragment thereof comprises a heavy chain variable region and a light chain variable region of an antibody, wherein the heavy chain variable region comprises HCDR1, HCDR2, HCDR3, wherein the light chain can be The variable regions comprise LCDR1, LCDR2, LCDR3, each of which has at least about 80% (e.g., about 85%, 90%, or 95%) sequence identity to HCDR1, HCDR2, HCDR3, LCDR1, LCDR2, LCDR3, respectively, of: Atezolizumab, Avelumab, BMS-936559, MPDL3280A (RG7446), or durvalumab (MEDI4736).

In certain embodiment, wherein the light chain variable regions each have at least about 80% (e.g., about 85%, 90%, or 95%) sequence identity to the light chain variable regions of: Atezolizumab, Avelumab, BMS-936559, MPDL3280A or durvalumab.

For example, the anti-PD-L1 antibody or antigen-binding fragment thereof comprises a heavy chain variable region and a light chain variable region of the antibody, each of which has at least about 80% (e.g., about 85%, 90%, or 95%) sequence identity: Atezolizumab, Avelumab, BMS-936559, MPDL3280A (RG7446), or durvalumab (MEDI4736).

In certain embodiment, wherein the anti-PD-L1 antibody or antigen-binding fragment thereof further comprises a light chain of the antibody, wherein the light chains are at least about 80% (e.g. about 85% identical) to the light chains of the following molecules, respectively, 90% or 95%) sequence identity: Atezolizumab, Avelumab, BMS-936559, MPDL3280A or durvalumab.

For example, the anti-PD-L1 antibody or antigen-binding fragment thereof comprises a heavy chain and a light chain of the antibody, each of which has at least about 80% (e.g. about 85%, 90%, or 95%) sequence identity: Atezolizumab, Avelumab, BMS-936559, MPDL3280A (RG7446), or durvalumab (MEDI4736).

In certain embodiment, wherein the anti-PD-L1 antibody or antigen-binding fragment thereof comprises: Atezolizumab, Avelumab, BMS-936559, MPDL3280A or durvalumab, or an antigen-binding fragment thereof, or a variant or biosimilar thereof, or its combination.

In certain embodiment, wherein the anti-PD-L1 antibody or antigen-binding fragment thereof comprises a heavy chain variable region of an antibody, wherein the heavy chain variable region comprises one or more heavy chain variable region CDRs (HCDRs) selected from the group consisting of: (a) HCDR1 having at least about 70% sequence identity to the amino acid sequence shown in SEQ ID NO:1; (b) having at least about 70% sequence identity to SEQ ID NO:2 HCDR2 of sequence identity; and (c) HCDR3 having at least about 70% sequence identity to SEQ ID NO:3.

In certain embodiment, wherein the anti-PD-L1 antibody or antigen-binding fragment thereof comprises an antibody heavy chain variable region, wherein the heavy chain variable region comprises one or more HCDRs selected from the group consisting of: (a) HCDR1 having the amino acid sequence shown in SEQ ID NO: 1 or having no more than 2 amino acid differences from the amino acid sequence shown in SEQ ID NO: 1 obtained through amino acid addition, elimination or substitution reactions HCDR1; (b) HCDR2 having the amino acid sequence shown in SEQ ID NO: 2 or HCDR2 having no more than 2 amino acid differences from the amino acid sequence shown in SEQ ID NO: 2 obtained through amino acid addition, elimination or substitution reactions; and (c) HCDR3 having the amino acid sequence shown in SEQ ID NO: 3 or HCDR3 having no more than 2 amino acid differences from the amino acid sequence shown in SEQ ID NO: 3 obtained through amino acid addition, elimination or substitution reactions.

In certain embodiment, wherein the anti-PD-L1 antibody or antigen-binding fragment thereof comprises a heavy chain variable region of an antibody, wherein the heavy chain variable region comprises HCDR1, HCDR2, HCDR3, the said HCDR1 comprises an amino acid sequence with at least about 70% sequence identity to the amino acid sequence shown in SEQ ID NO: 1, said HCDR2 comprises an amino acid sequence with at least about 70% sequence identity to the amino acid sequence shown in SEQ ID NO: 2, wherein the HCDR3 comprises an amino acid sequence having at least about 70% sequence identity to the amino acid sequence set forth in SEQ ID NO:3.

In certain embodiment, wherein the anti-PD-L1 antibody or antigen-binding fragment thereof comprises a heavy chain variable region of an antibody, wherein the heavy chain variable region comprises HCDR1, HCDR2, HCDR3, the said HCDR1 comprises the amino acid sequence shown in SEQ ID NO: 1 or an amino acid sequence obtained by amino acid addition, elimination or substitution reaction with no more than 2 amino acid differences from the amino acid sequence shown in SEQ ID NO: 1; HCDR2 comprises the amino acid sequence shown in SEQ ID NO: 2 or an amino acid sequence obtained by amino acid addition, elimination or substitution reaction with no more than 2 amino acid differences from the amino acid sequence shown in SEQ ID NO: 2; the HCDR3 comprising the amino acid sequence shown in SEQ ID NO: 3 or an amino acid sequence obtained by amino acid addition, elimination or substitution reaction and having no more than 2 amino acid differences from the amino acid sequence shown in SEQ ID NO: 3.

In certain embodiment, wherein the anti-PD-L1 antibody or antigen-binding fragment thereof comprises an antibody heavy chain variable region, wherein the heavy chain variable region comprises an amino acid sequence selected from the group consisting of: (a) the amino acid sequence set forth in SEQ ID NO: 4; (b) an amino acid sequence at least about 85%, 90%, 95% or 99% sequence identity to the amino acid sequence set forth in SEQ ID NO: 4; and (c) an amino acid sequence having 1 or more differences from the amino acid sequence shown in SEQ ID NO: 4 obtained by addition, elimination or substitution reaction.

In certain embodiment, wherein the TGFβ inhibitor binds to TGFβ1, TGFβ2 or TGFβ3.

In certain embodiment, wherein the TGFβ inhibitor binds to TGFβ or transforming growth factor β receptor (TGFβR).

In certain embodiment, wherein the TGFβ inhibitor binds to TGFβ type I receptor (TGFβRI), TGFβ type II receptor (TGFβRII) or TGFβ type III receptor (TGFβRIII).

In certain embodiment, wherein the TGFβ inhibitor comprises: 1) an anti-TGFβ antibody or an antigen-binding fragment thereof; 2) an anti-TGFβR antibody or an antigen-binding fragment thereof; 3) small molecule TGFβinhibitors; 4) proteins comprising transforming growth factor β receptor II (TGFβRII) or functionally active fragments thereof, or variants or biosimilars thereof, or combinations thereof.

In certain embodiment, wherein said anti-TGFβ antibody or antigen-binding fragment thereof comprises an antibody heavy chain variable region, wherein said heavy chain variable region comprises HCDR1, HCDR2, HCDR3, each of which has at least about 80% sequence identity with HCDR1, HCDR2, and HCDR3 of the fresolimumab molecule.

In certain embodiment, wherein the anti-TGFβ antibody or its antigen-binding fragment comprises an antibody heavy chain variable region, wherein the heavy chain variable region has at least about 80% sequence identity to the heavy chain variable region of Fresolimumab.

In certain embodiment, wherein the anti-TGFβ antibody or its antigen-binding fragment comprises an antibody heavy chain, wherein the heavy chain has at least about 80% sequence identity to the heavy chain of Fresolimumab.

In certain embodiment, wherein said anti-TGFβ antibody or antigen-binding fragment thereof comprises an antibody's light chain variable region, wherein said light chain variable region comprises LCDR1, LCDR2, LCDR3, each of which has at least about 80% sequence identity with LCDR1, LCDR2, and LCDR3 of the Fresolimumab.

In certain embodiment, wherein the anti-TGFβ antibody or its antigen-binding fragment comprises an antibody light chain variable region, wherein the light chain variable region has at least about 80% sequence identity to the light chain variable region.

In certain embodiment, wherein the anti-TGFβ antibody, or antigen-binding fragment thereof, comprises a light chain of an antibody, wherein the light chain has at least about 80% sequence identity with the light chain of a Fresolimumab molecule.

In certain embodiment, wherein the anti-TGFβ antibody or antigen-binding fragment thereof comprises: Fresolimumab, or an antigen-binding fragment thereof, or a variant or biosimilar thereof.

In certain embodiment, wherein the small molecule TGFβ inhibitor comprises the following compound: SB525334, SD-208, SB431542, LY2109761, LY2157299, GW788388, RepSox, SIS3, LDN-193189, EW-7197, LY364947, or a combination thereof.

In certain embodiment, wherein the TGFβRII comprises a polypeptide having a wild-type human TGFβ receptor type 2 isoform A sequence(For example, the amino acid sequence of NCBI reference sequence (Ref Seq) accession number NP_001020018), or a polypeptide having a wild-type human TGFβ receptor type 2 isoform B sequence(For example, the amino acid sequence of NCBI reference sequence (Ref Seq) accession number NP_003233), or a polypeptide having a sequence substantially identical to an amino acid sequence thereof.

In certain embodiment, wherein said TGFβ RII or its functionally active fragment comprises: human TGFβRII extracellular domain (ECD), any part of NCBI Ref Seq NO. NP_001020018 or NCBI Ref Seq NO. NP_003233, or an amino acid sequence substantially identical to sequence.

In certain embodiment, wherein the TGFβRII or a functionally active fragment thereof comprises:
(a) the amino acid sequence of SEQ ID NO: 6;
(b) an amino acid sequence having at least about 85% sequence identity to the amino acid sequence set forth in SEQ ID NO: 6; Or
(c) has an amino acid sequence in which one or more amino acids are added, deleted and/or substituted as compared to the amino acid sequence shown in SEQ ID NO: 6.

In certain embodiment, wherein the bifunctional immune checkpoint/TGFβ inhibitor is a fusion protein.

In certain embodiment, wherein the PD-L1/TGFβ dual inhibitor or PD-1/TGFβ dual inhibitor is a fusion protein.

In certain embodiment, wherein the PD-L1/TGFβ dual inhibitor comprises a PD-L1 targeting moiety and a TGFβ receptor domain comprising transforming growth factor beta receptor II (TGF β RII) or a functionally active fragment thereof.

In certain embodiment, wherein the PD-L1//TGFβ dual inhibitor comprises a polypeptide, wherein the polypeptide comprises at least: (i) a heavy chain variable region of an anti-PD-L1 antibody; and (ii) TGFβII or a functionally active fragment thereof.

In certain embodiment, wherein the anti-PD-L1 antibody heavy chain variable region comprises HCDR1, HCDR2, HCDR3; Wherein the HCDR1 comprises an amino acid sequence having at least about 70% sequence identity to the amino acid sequence set forth in SEQ ID NO: 1, the HCDR2 comprises an amino acid sequence having at least about 70% sequence identity to the amino acid sequence set forth in SEQ ID NO: 2, and the HCDR3 comprises an amino acid sequence having at least about 70% sequence identity to the amino acid sequence set forth in SEQ ID NO: 3; or wherein the HCDR1, HCDR2, HCDR3 have at least about 80% sequence identity to the HCDR1, HCDR2, HCDR3, respectively, of Atezolizumab, Avelumab, BMS-936559, MPDL3280A (RG7446), or durvalumab (MEDI-4736).

In certain embodiment, wherein the anti-PD-L1 antibody heavy chain variable region comprises HCDR1, HCDR2, HCDR3, and the HCDR1 comprises the amino acid sequence shown in SEQ ID NO: 1 or an amino acid sequence obtained by amino acid addition, elimination or substitution reaction and having no more than 2 amino acid differences from the amino acid sequence shown in SEQ ID NO: 1; The HCDR2 comprises the amino acid sequence represented by SEQ ID NO: 2 or an amino acid sequence obtained by amino acid addition, elimination or substitution reaction and having no more than 2 amino acid differences from the amino acid sequence represented by SEQ ID NO: 2; the HCDR3 comprises an amino acid sequence represented by SEQ ID NO: 3 or an amino acid sequence obtained by an amino acid addition, elimination or substitution reaction and having no more than 2 amino acid differences from the amino acid sequence represented by SEQ ID NO: 3.

In certain embodiment, wherein the anti-PD-L1 antibody heavy chain variable region comprises an amino acid sequence selected from the group consisting of: (a) the amino acid sequence set forth in SEQ ID NO: 4; (b) an amino acid sequence having at least about 85%, 90%, 95%, or 99% sequence identity to the amino acid sequence set forth in SEQ ID NO: 4; (c) an amino acid sequence having 1 or more differences from the amino acid sequence shown in SEQ ID NO: 4 obtained by addition, elimination or substitution; And (d) an amino acid sequence having at least about 80% sequence identity to the heavy chain variable region of Atezolizumab, Avelumab, BMS-936559, MPDL3280A, or durvalumab.

In certain embodiment, wherein the TGFβRII or a functionally active fragment thereof comprises:
(a) the amino acid sequence of SEQ ID NO: 6;
(b) an amino acid sequence having at least about 85% sequence identity to the amino acid sequence set forth in SEQ ID NO: 6; Or
(c) has an amino acid sequence in which one or more amino acids are added, deleted and/or substituted as compared to the amino acid sequence shown in SEQ ID NO: 6.

In certain embodiment, wherein the polypeptide further comprises a linker that links the C-terminus of the heavy chain variable region of the anti-PD-L1 antibody or antigen-binding fragment thereof to the N-terminus of the TGFβRII or functionally active fragment thereof.

In certain embodiment, said polypeptide further comprising CH2, CH3 domains, said polypeptide comprising the heavy chain variable region (VH) of anti-PD-L1 antibody, CH2, CH3 domains and TGF ORB or a functionally active fragment thereof in sequence from N-terminus to C-terminus, said CH3 domain having its C-terminus connected to the N-terminus of said TGF ORB or a functionally active fragment thereof by a linker.

In certain embodiment, wherein the CH2, CH3 domains are derived from IgG. For example, said CH2, CH3 are derived from IgG1, IgG2, IgG3 or IgG4.

In certain embodiment, wherein the linker is a peptide linker.

In certain embodiment, wherein the amino acid sequence of the peptide linker is (G₄S)ₓ, wherein x is any integer from 3 to 6.

In certain embodiment, wherein the peptide linker comprises an amino acid sequence selected from the group consisting of: (a) the amino acid sequence set forth in SEQ ID NO: 5; (b) an amino acid sequence having at least about 85%, 90%, 95%, or 99% sequence identity to the amino acid sequence set forth in SEQ ID NO: 5; And (c) an amino acid sequence having 1 or more differences from the amino acid sequence shown in SEQ ID NO: 5 obtained by addition, elimination or substitution reaction.

In certain embodiment, wherein the polypeptide comprises an amino acid sequence selected from the group consisting of: (a) the amino acid sequence set forth in SEQ ID NO: 7; (b) an amino acid sequence having at least about 85%, 90%, 95%, or 99% sequence identity to the amino acid sequence set forth in SEQ ID NO: 7; (c) an amino acid sequence having 1 or more differences from the amino acid sequence shown in SEQ ID NO: 7 obtained by addition, elimination or substitution reactions.

In certain embodiment, wherein the PD-L1//TGFβ dual inhibitor comprises two aforementioned polypeptides.

In certain embodiment, wherein the PD-L1/TGFβ dual inhibitor comprises a first polypeptide and a second polypeptide, wherein the first polypeptide comprises at least: (i) a heavy chain of an anti-PD-L1 antibody variable region; and (ii) TGFβRII or a functionally active fragment thereof; wherein the second polypeptide comprises at least the light chain variable region of an anti-PD-L1 antibody; wherein the heavy chain variable region of the first polypeptide and the light chain variable regions of the second polypeptide are capable of specifically binding to PD-L1.

In certain embodiment, wherein the first polypeptide further comprises a linker, said linker connects the C-terminus of the heavy chain variable region of the anti-PD-L1 antibody or antigen-binding fragment thereof with the TGFβRII or the N-terminus of its functionally active fragments.

In certain embodiment, wherein the PD-L1/TGFβ dual inhibitor comprises a first polypeptide and a second polypeptide, wherein, the first polypeptide comprises the heavy chain variable region (VH) of the anti-PD-L1 antibody, the CH1 domain, and TGFβRII or its functionally active fragment sequentially from the N-terminus to the C-terminus, and the C-terminus of the CH1 domain is connected to the TGFβRII or its functionally active fragment N-terminus is connected by a linker; and the second polypeptide comprises a light chain variable region (VL) and a light chain constant region (CL) of an anti-PD-L1 antibody from the N-terminus to the C-terminus).

In certain embodiment, wherein the said linker is peptide linker.

In certain embodiment, wherein the first polypeptide has at least about 80% sequence identity to a polypeptide having the same function as: M7824 or SHR-1701.

In certain embodiment, wherein the second polypeptide has at least about 80% sequence identity to a polypeptide having the same function as: M7824 or SHR-1701.

In certain embodiment, wherein the PD-L1/TGFβ dual inhibitor comprises M7824, SHR-1702, or variant or biosimilar or their combination.

In certain embodiment, wherein the PI3K inhibitor is PI3Kδ/γ dual inhibitor.

In certain embodiment, wherein the said PI3K inhibitors comprise compounds as formula (IA-I), (IA-II), (IA-III), (IA-IV):

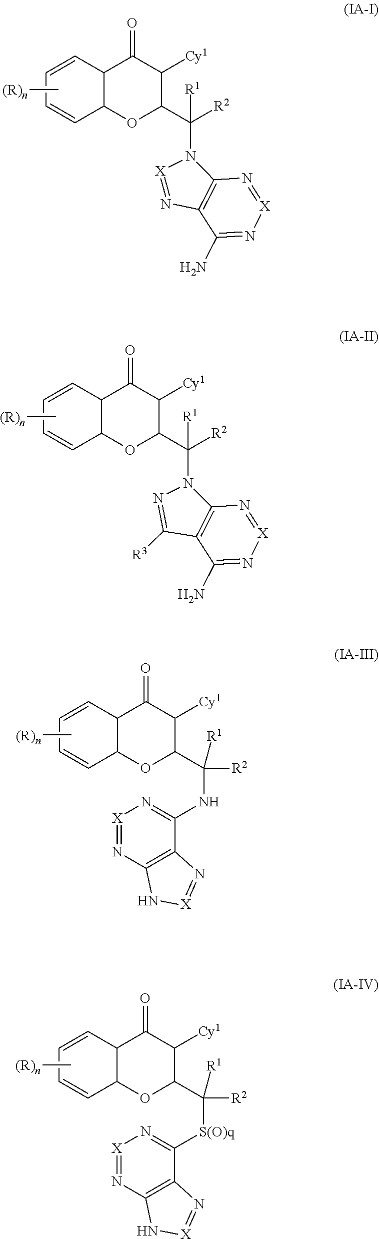

or their enantiomers, mixtures of enantiomers, mixtures of two or more diastereoisomers, isotopic variants, pharmaceutically acceptable salts, solvates, hydrates or prodrug, wherein,
each occurrence of R is independently selected from hydrogen, halogen, —OR^{a}, CN, substituted or unsubstituted C₁₋₆ alkyl, substituted or unsubstituted C₂₋₆ alkenyl, substituted or unsubstituted C₂₋₆ alkynyl, substituted or unsubstituted C₃₋₈ cycloalkyl, and substituted or unsubstituted heterocyclic group;
R¹ and R² may be the same or different and are independently selected from hydrogen, halogen, and substituted or unsubstituted C₁₋₆ alkyl, or both R¹ and R² directly bound to a common atom, may be joined to form an oxo group (═O) or a substituted or unsubstituted saturated or unsaturated 3-10 member ring, including the carbon atom to which R¹ and R² are bound, which may optionally include one or more heteroatoms which may be the same or different and are selected from O, NR^{a} and S;
Cy¹ is selected from substituted or unsubstituted C₃₋₈ cycloalkyl, substituted or unsubstituted 3 to 15 membered heterocyclic groups having at least one heteroatom selected from N, O and S, substituted or unsubstituted C₆₋₂₀ aryl and substituted or unsubstituted 5 to 14 membered heteroaryl monocyclic groups with one or more heteroatoms selected from N, O and S;
each occurrence of R^{a} may be the same or different and are independently selected from hydrogen, halogen, hydroxy, cyano, substituted or unsubstituted C₁₋₆alkyl, —NR^{c}R^{d}, wherein R^{c} and R^{d} are independently hydrogen, halogen, hydroxy, cyano, substituted or unsubstituted C₁₋₆alkyl, and C₁₋₆alkoxy, and —OR^{c}, wherein R^{c} is substituted or unsubstituted C₁₋₆alkyl,
n is an integer from 1 to 4;
q is 0, 1 or 2;
each occurrence of X is independently selected from CR³ or N;
and each occurrence of R³ is independently selected from hydrogen, hydroxy, halogen, carboxyl, cyano, nitro, substituted or unsubstituted C₁₋₈ alkyl, substituted or unsubstituted C₁₋₈ alkoxy, substituted or unsubstituted C₂₋₁₀ alkenyl, substituted or unsubstituted C₂₋₁₀ alkynyl, substituted or unsubstituted C₆₋₂₀ aryl, substituted or unsubstituted C₆₋₂₀ aryl C₁₋₈alkyl, substituted or unsubstituted C₃₋₂₀ cycloalkyl,
substituted or unsubstituted C₃₋₂₀ cycloalkyl C₁₋₈ alkyl, substituted or unsubstituted C₃₋₈ cycloalkenyl C₁₋₈ alkyl, substituted or unsubstituted C₃₋₈ cycloalkene, substituted or unsubstituted 5-14-membered heteroaryl with one or more heteroatoms selected from N, O and S, substituted or unsubstituted with one or more heteroatoms selected from N, O, S 5-14 membered heteroaryl C₁-8 alkyl, substituted or unsubstituted 3 to 15 membered heterocycles having at least one heteroatom selected from N, O and S, substituted or unsubstituted 3 to 15 membered heterocyclyl C₁₋₈ alkyl rings having at least one heteroatom selected from N, O and S, substituted or unsubstituted guanidine, —COOR^{x}, —C(O)R^{x}, —C(S)R^{x}, —C(O)NR^{x}R^{y}, —C(O)ONR^{x}R^{y}, —NR^{y}R^{z}, —NR^{x}CONR^{y}R^{z}, —N(R^{x})SOR^{y}, —N(R^{x})SO₂R^{y}, —(═N—N(R^{x})R^{y}), —NR^{x}C(O)OR^{y}, —NR^{x}R^{y}, —NR^{x}C(O)R^{y}—, —NR^{x}C(S)R^{y}—NR^{x}C(S)NR^{y}R^{z}, —SONR^{x}R^{y}—, —S0₂NR^{x}R^{y}—, —OR^{x}, —OR^{x}C(O)NR^{y}R^{z}, —OR^{x}C(O)OR^{y}—, —OC(O)R^{x}, —OC(O)NR^{x}R^{y}, —R^{x}NR^{y}C(O)R^{z}, —R^{x}OR^{y}, —R^{x}C(O)OR^{y}, —R^{x}C(O)NR^{y}R^{z}, —R^{x}C(O)R^{x}, —R^{x}OC(O)R^{y}, —SR^{x}, —SOR^{x}, —SO₂R^{x}, —ONO₂,
wherein R^{x}, R^{y} and R^{z} in each of the above groups can be hydrogen, substituted or unsubstituted C¹⁻⁸ alkyl, substituted or unsubstituted C₁₋₈ alkoxy, substituted or unsubstituted C₂₋₁₀ alkenyl, substituted or unsubstituted C₂₋₁₂ alkynyl, substituted or unsubstituted C₆₋₂₀ aryl, substituted or unsubstituted C₆₋₂₀ aryl C₁₋₈ alkyl, substituted or unsubstituted C₃₋₂₀ cycloalkyl, substituted or unsubstituted C₃₋₂₀ cycloalkylC₁₋₈ alkyl, substituted or unsubstituted C₃₋₈ cycloalkenyl, substituted or unsubstituted 5 to 14 membered heteroaryl with one or more heteroatoms selected from N, O and S, substituted or unsubstituted heteroaryl with one or more heteroatoms selected from N, O and S atomic 5 to 14 membered heteroaryl C₁-8 alkyl, substituted or unsubstituted 3 to 15 membered heterocycle having at least one heteroatom selected from N, O and S, substituted or unsubstituted 3 to 15 membered heterocyclyl C₁₋₈ alkyl ring having at least one heteroatom selected from N, O and S or substituted or unsubstituted amino, or any two of R^{x}, R^{y} and R^{z} may be joined to form a substituted or unsubstituted saturated or unsaturated 3-10 membered ring, which may optionally include heteroatoms which may be the same or different and are selected from O, NR^{x} or S, wherein R^{x} is hydrogen or substituted or unsubstituted alkyl.

In certain embodiment, wherein the PI3K inhibitor comprises compounds as formula (IA-V):

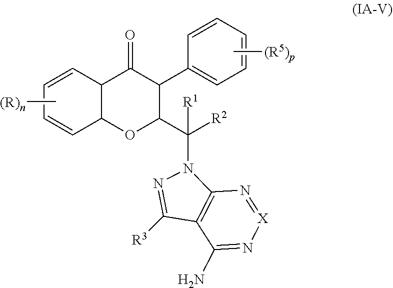

or their enantiomers, mixtures of enantiomers, mixtures of two or more diastereoisomers, isotopic variants, pharmaceutically acceptable salts, solvates, hydrates or prodrug, wherein
each occurrence of R is independently selected from hydrogen, halogen, —OR^{a}, CN, substituted or unsubstituted C₁₋₆ alkyl, substituted or unsubstituted C₂₋₆ alkenyl, substituted or unsubstituted C₂₋₆ alkynyl, substituted or unsubstituted C₃₋₈ cycloalkyl, and substituted or unsubstituted heterocyclic group;
R¹ and R² may be the same or different and are independently selected from hydrogen, halogen, and substituted or unsubstituted C₁₋₆ alkyl, or both R¹ and R² directly bound to a common atom, may be joined to form an oxo group (═O) or a substituted or unsubstituted saturated or unsaturated 3-10 member ring, including the carbon atom to which R¹ and R² are bound, which may optionally include one or more heteroatoms which may be the same or different and are selected from O, NR^{a} and S;
each occurrence of X is independently selected from CR³ or N; and
each occurrence of R³ is independently selected from hydrogen, hydroxy, halogen, carboxyl, cyano, nitro, substituted or unsubstituted C₁₋₈ alkyl, substituted or unsubstituted C₁₋₈ alkoxy, substituted or unsubstituted C₂₋₁₀ alkenyl, substituted or unsubstituted C₂₋₁₀ alkynyl, substituted or unsubstituted C₆₋₂₀ aryl, substituted or unsubstituted C₆₋₂₀ aryl C₁₋₈ alkyl, substituted or unsubstituted C₃₋₂₀ cycloalkyl, substituted or unsubstituted C₃₋₂₀ cycloalkyl C₁₋₈ alkyl, substituted or unsubstituted C₃₋₈ cycloalkenyl C₁₋₈ alkyl, substituted or unsubstituted C₃₋₈ cycloalkene, substituted or unsubstituted 5-14-membered heteroaryl with one or more heteroatoms selected from N, O and S, substituted or unsubstituted with one or more heteroatoms selected from N, O, S 5-14 membered heteroaryl C₁₋₈ alkyl, substituted or unsubstituted 3 to 15 membered heterocycles having at least one heteroatom selected from N, O and S, substituted or unsubstituted 3 to 15 membered heterocyclyl C₁₋₈ alkyl rings having at least one heteroatom selected from N, O and S, substituted or unsubstituted guanidine, —COOR^{x}, —C(O)R^{x}, —C(S)R^{x}, —C(O)NR^{x}R^{y}, —C(O)ONR^{x}R^{y}, —NR^{y}R^{z}, —NR^{x}CONR^{y}R^{z}, —N(R^{x})SOR^{y}, —N(R^{x})SO₂R^{y}, —(═N—N(R^{x})R^{y}), —NR^{x}C(O)OR^{y}, —NR^{x}R^{y}, —NR^{x}C(O)R^{y}—, —NR^{x}C(S)R^{y}—NR^{x}C(S)NR^{y}R^{z}, —SONR^{x}R^{y}—, —SO₂NR^{x}R^{y}—, —OR^{x}, —OR^{x}C(O)NR^{y}R^{z}, —OR^{x}C(O)OR^{y}—, —OC(O)R^{x}, —OC(O)NR^{x}R^{y}, —R^{x}NR^{y}C(O)R^{z}, —R^{x}OR^{y}, —R^{x}C(O)OR^{y}, —R^{x}C(O)NR^{y}R^{z}, —R^{x}C(O)R^{x}, —R^{x}OC(O)R^{y}, —SR^{x}, —SOR^{x}, —SO₂R^{x}, —ONO₂,
wherein R^{x}, R^{y} and R^{z} in each of the above groups can be hydrogen, substituted or unsubstituted C₁₋₈ alkyl, substituted or unsubstituted C₁₋₈ alkoxy, substituted or unsubstituted C₂₋₁₀ alkenyl, substituted or unsubstituted C₂₋₁₂ alkynyl, substituted or unsubstituted C₆₋₂₀ aryl, substituted or unsubstituted C₆₋₂₀ aryl C₁₋₈ alkyl, substituted or unsubstituted C₃₋₂₀ cycloalkyl, substituted or unsubstituted C₃₋₂₀ cycloalkylC₁₋₈ alkyl, substituted or unsubstituted C₃₋₈ cycloalkenyl, substituted or unsubstituted 5 to 14 membered heteroaryl with one or more heteroatoms selected from N, O and S, substituted or unsubstituted heteroaryl with one or more heteroatoms selected from N, O and S atomic 5 to 14 membered heteroaryl C₁₋₈ alkyl, substituted or unsubstituted 3 to 15 membered heterocycle having at least one heteroatom selected from N, O and S, substituted or unsubstituted 3 to 15 membered heterocyclyl C₁₋₈ alkyl ring having at least one heteroatom selected from N, O and S or substituted or unsubstituted amino, or any two of R^{x}, R^{y} and R^{z} may be joined to form a substituted or unsubstituted saturated or unsaturated 3-10 membered ring, which may optionally include heteroatoms which may be the same or different and are selected from O, NR^{x} or S, wherein R^{x} is hydrogen or substituted or unsubstituted alkyl,
each occurrence of R⁵ is hydrogen, C₁₋₆ alkyl or halogen,
n is 0, 1, 2, 3 or 4; and
P is 0, 1, 2, 3, 4 or 5.

In certain embodiment, wherein the PI3K inhibitor comprises compounds as formula (IA-VI):

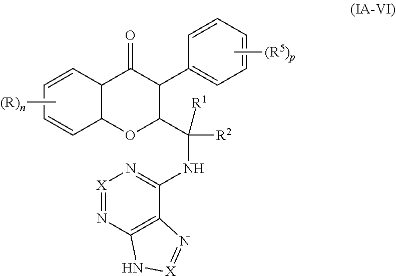

or their enantiomers, mixtures of enantiomers, mixtures of two or more diastereoisomers, isotopic variants, pharmaceutically acceptable salts, solvates, hydrates or prodrug, wherein
each occurrence of R is independently selected from hydrogen, halogen, —OR^{a}, CN, substituted or unsubstituted C₁₋₆ alkyl, substituted or unsubstituted C₂₋₆ alkenyl, substituted or unsubstituted C₂₋₆ alkynyl, substituted or unsubstituted C₃₋₈ cycloalkyl, and substituted or unsubstituted heterocyclic group;
R¹ and R² may be the same or different and are independently selected from hydrogen, halogen, and substituted or unsubstituted C₁₋₆ alkyl, or both R¹ and R² directly bound to a common atom, may be joined to form an oxo group (═O) or a substituted or unsubstituted saturated or unsaturated 3-10 member ring, including the carbon atom to which R¹ and R² are bound, which may optionally include one or more heteroatoms which may be the same or different and are selected from O, NR^{a} and S;
each occurrence of X is independently selected from CR³ or N; and
each occurrence of R³ is independently selected from hydrogen, hydroxy, halogen, carboxyl, cyano, nitro, substituted or unsubstituted C₁₋₈ alkyl, substituted or unsubstituted C₁₋₈ alkoxy, substituted or unsubstituted C₂₋₁₀ alkenyl, substituted or unsubstituted C₂₋₁₀ alkynyl, substituted or unsubstituted C₆₋₂₀ aryl, substituted or unsubstituted C₆₋₂₀ aryl C₁₋₈alkyl, substituted or unsubstituted C₃₋₂₀ cycloalkyl, substituted or unsubstituted C₃₋₂₀ cycloalkyl C₁₋₈ alkyl, substituted or unsubstituted C₃₋₈ cycloalkenyl C₁₋₈ alkyl, substituted or unsubstituted C₃₋₈ cycloalkene, substituted or unsubstituted 5-14-membered heteroaryl with one or more heteroatoms selected from N, O and S, substituted or unsubstituted with one or more heteroatoms selected from N, O, S 5-14 membered heteroaryl C₁₋₈ alkyl, substituted or unsubstituted 3 to 15 membered heterocycles having at least one heteroatom selected from N, O and S, substituted or unsubstituted 3 to 15 membered heterocyclyl C₁₋₈ alkyl rings having at least one heteroatom selected from N, O and S, substituted or unsubstituted guanidine, —COOR^{x}, —C(O)R^{x}, —C(S)R^{x}, —C(O)NR^{x}R^{y}, —C(O)ONR^{x}R^{y}, —NR^{y}R^{z}, —NR^{x}CONR^{y}R^{z}, —N(R^{x})SOR^{y}, —N(R^{x})SO₂R^{y}, —(═N—N(R^{x})R^{y}), —NR^{x}C(O)OR^{y}, —NR^{x}R^{y}, —NR^{x}C(O)R^{y}—, —NR^{x}C(S)R^{y}—NR^{x}C(S)NR^{y}R^{z}, —SONR^{x}R^{y}—, —SO₂NR^{x}R^{y}—, —OR^{x}, —OR^{x}C(O)NR^{y}R^{z}, —OR^{x}C(O)OR^{y}—, —OC(O)R^{x}, —OC(O)NR^{x}R^{y}, —R^{x}NR^{y}C(O)R^{z}, —R^{x}OR^{y}, —R^{x}C(O)OR^{y}, —R^{x}C(O)NR^{y}R^{z}, —R^{x}C(O)R^{x}, —R^{x}OC(O)R^{y}, —SR^{x}, —SOR^{x}, —SO₂R^{x}, —ONO₂,
wherein R^{x}, R^{y} and R^{z} in each of the above groups can be hydrogen, substituted or unsubstituted C¹⁻⁸ alkyl, substituted or unsubstituted C₁₋₈ alkoxy, substituted or unsubstituted C₂₋₁₀ alkenyl, substituted or unsubstituted C₂₋₁₂ alkynyl, substituted or unsubstituted C₆₋₂₀ aryl, substituted or unsubstituted C₆₋₂₀ aryl C₁₋₈ alkyl, substituted or unsubstituted C₃₋₂₀ cycloalkyl, substituted or unsubstituted C₃₋₂₀ cycloalkylC₁₋₈ alkyl, substituted or unsubstituted C₃₋₈ cycloalkenyl, substituted or unsubstituted 5 to 14 membered heteroaryl with one or more heteroatoms selected from N, O and S, substituted or unsubstituted heteroaryl with one or more heteroatoms selected from N, O and S atomic 5 to 14 membered heteroaryl C₁₋₈ alkyl, substituted or unsubstituted 3 to 15 membered heterocycle having at least one heteroatom selected from N, O and S, substituted or unsubstituted 3 to 15 membered heterocyclyl C₁₋₈ alkyl ring having at least one heteroatom selected from N, O and S or substituted or unsubstituted amino, or any two of R^{x}, R^{y} and R^{z} may be joined to form a substituted or unsubstituted saturated or unsaturated 3-10 membered ring, which may optionally include heteroatoms which may be the same or different and are selected from O, NR^{x} or S, wherein R^{x} is hydrogen or substituted or unsubstituted alkyl,
each occurrence of R⁵ is hydrogen, C₁₋₆ alkyl or halogen,
n is 0, 1, 2, 3 or 4; and
P is 0, 1, 2, 3, 4 or 5.

In certain embodiment, wherein R is hydrogen, halogen, substituted or unsubstituted C₁₋₆ alkyl or OR^{a}.

In certain embodiment, wherein R^{a} is alkyl.

In certain embodiment, wherein Cy¹ is selected from:

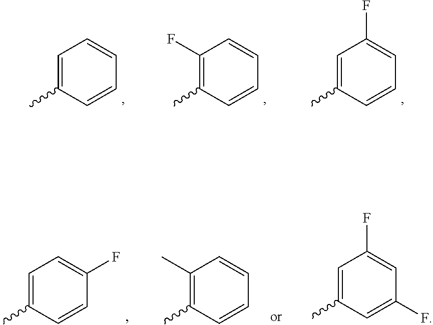

In certain embodiment, wherein R¹ and R² each independently represent hydrogen or substituted or unsubstituted C₁₋₆ alkyl.

In certain embodiment, wherein R³ is iodo, cyano, substituted unsubstituted alkyl, substituted or unsubstituted alkynyl, substituted or unsubstituted aryl, substituted or unsubstituted heteroaryl.

In certain embodiment, wherein X is CR³ and each occurrence of R³ is independently hydrogen, halogen, hydroxyl or NH₂.

In certain embodiment, wherein R³ is hydrogen.

In certain embodiment, wherein said PI3K inhibitors comprise:
2-[(6-Amino-9H-purin-9-yl) methyl]-6-bromo-3-phenyl-4H-chromen-4-one;
6-Bromo-2-(morpholinomethyl)-3-phenyl-4H-chromen-4-one;
6-Bromo-2-(morpholinomethyl)-3-phenyl-4H-chromen-4-one hydrochloride;
2-[(6-Amino-9H-purin-9-yl) methyl]-3-phenyl-4H-chromen-4-one;
2-(Morpholinomethyl)-3-phenyl-4H-chromen-4-one;
2-(Morpholinomethyl)-3-phenyl-4H-chromen-4-one hydrochloride;
2-[(1H-Benzo[d]imidazol-1-yl) methyl]-6-bromo-3-phenyl-4H-chromen-4-one;
6-Bromo-2-[(4-methyl-1H-benzo[d]imidazol-1-yl) methyl]-3-phenyl-4H-chromen-4-one;
2-[(1 H-benzo [d] imidazol-1-yl) methyl]-3-phenyl-4H-chromen-4-one;
2-[(4-methyl-1 H-benzo [d] imidazol-1-yl)methyl]-3-phenyl-4H-chromen-4-one;
2-[(6-chloro-9H-purin-9-yl)methyl]-3-phenyl-4H-chromen-4-one;
6-Bromo-2-[(6-chloro-9H-purin-9-yl)methyl]-3-phenyl-4H-chromen-4-one;
2-((9H-Purin-6-ylthio)methyl)-3-phenyl-4H-chromen-4-one;
2-[(1H-Imidazol-1-yl)methyl]-3-phenyl-4H-chromen-4-one;
2-[(9H-Purin-6-ylthio)methyl]-6-bromo-3-phenyl-4H-chromen-4-one;
2-((4-Amino-1H-pyrazolo[3,4-d]pyrimidin-1-yl)methyl)-6-bromo-3-phenyl-4H-chromen-4-one;
2-[(6-Amino-9H-purin-9-yl)methyl]-6-bromo-3-(4-fluorophenyl)-4H-chromen-4-one;
2-[(6-Arnino-9H-purin-9-yl)methyl]-3-(4-fluorophenyl)-4H-chromen-4-one; 6-Bromo-3-(4-fluorophenyl)-2-(morpholinomethyl)-4H-chromen-4-one; 6-Bromo-3-(4-fluorophenyl)-2-(morpholinomethyl)-4H-chromen-4-one hydro chloride;
3-(4-fluorophenyl)-2-(morpholinomethyl)-4H-chromen-4-one; 3-(4-fluorophenyl)-2-(morpholinomethyl)-4H-chromen-4-one hydrochloride;
2-[(6-Amino-9H-purin-9-yl)methyl]-6-bromo-3-o-tolyl-4H-chromen-4-one;
7-[(6-Bromo-4-oxo-3-phenyl-4H-chromen-2-yl)methyl]-1,3-dimethyl-1H-purine-2,6(3H,7H)-dione;
2-(1-(6-Amino-9H-purin-9-yl)ethyl)-6-bromo-3-phenyl-4H-chromen-4-one;
2-(1-(9H-Purin-6-ylthio)ethyl)-6-bromo-3-phenyl-4H-chromen-4-one;
2-(1-(6-Amino-9H-purin-9-yl)ethyl)-3-phenyl-4H-chromen-4-one;
(S)-2-(1-(9H-purin-6-ylamino)ethyl)-6-bromo-3-phenyl-4H-chromen-4-one;
2-((9H-purin-6-ylamino)methyl)-6-bromo-3-phenyl-4H-chromen-4-one;
2-(1-(4-amino-1 H-pyrazolo [3,4-d]pyrimidin-1-yl)ethyl)-6-bromo-3-phenyl-4H-chromen-4-one;
2-((6-Amino-9H-purin-9-yl)methyl)-6-methoxy-3-phenyl-4H-chromen-4-one;
2-(1-(6-Amino-9H-purin-9-yl)ethyl)-6-bromo-3-(2-fluorophenyl)-4H-chromen-4-one;
2-((6-Amino-9H-purin-9-yl)methyl)-6-bromo-3-(2-fluorophenyl)-4H-chromen-4-one;
2-(1-(4-Amino-1 H-pyrazolo [3,4-d]pyrimidin-1-yl)ethyl)-3-phenyl-4H-chromen-4-one;
2-(1-(6-Amino-9H-purin-9-yl) propyl)-3-phenyl-4H-chromen-4-one; 2-(1-(6-Amino-9H-purin-9-yl)ethyl)-3-(3-fluorophenyl)-4H-chromen-4-one;
2-((6-Amino-9H-purin-9-yl)methyl)-3-(2-fluorophenyl)-4H-chromen-4-one;
2-(1-(6-Amino-9H-purin-9-yl)ethyl)-3-(2-fluorophenyl)-4H-chromen-4-one;
2-(1-(6-Amino-9H-purin-9-yl)propyl)-3-(2-fluorophenyl)-4H-chromen-4-one;
2-(1-(6-amino-9H-purin-9-yl)propyl)-3-(3-fluorophenyl)-4H-chromen-4-one;
2-(1-(6-Amino-9H-purin-9-yl)propyl)-3-(4-fluorophenyl)-4H-chromen-4-one;
2-(1-(6-amino-9H-purin-9-yl)propyl)-6-fluoro-3-phenyl-4H-chromen-4-one;
2-(1-(6-Amino-9H-purin-9-yl)ethyl)-3-(4-fluorophenyl)-4H-chromen-4-one;
2-(1-(6-Amino-9H-purin-9-yl)ethyl)-6-fluoro-3-phenyl-4H-chromen-4-one;
2-(1-(4-Amino-3-(3-methoxyphenyl)-1H-pyrazolo[3,4-d]pyrimidin-1-yl)ethyl)-3-phenyl-4H-chromen-4-one;
2-(1-(4-Amino-3-(3-hydroxyphenyl)-1 H-pyrazolo [3,4-d]pyrimidin-1-yl)ethyl)-3-phenyl-4H-chromen-4-one;
2-((9H-purin-6-ylamino)methyl)-3-phenyl-4H-chromen-4-one;
2-(1-(6-Amino-9H-purin-9-yl)ethyl)-3-o-tolyl-4H-chromen-4-one;
2-((9H-purin-6-ylamino)methyl)-3-(2-fluorophenyl)-4H-chromen-4-one;
2-((9H-purin-6-ylamino)methyl)-3-(3-fluorophenyl)-4H-chromen-4-one;
(S)-2-(1-(9H-purin-6-ylamino)ethyl)-3-(3-fluorophenyl)-4H-chromen-4-one;
2-(1-(6-amino-9H-purin-9-yl)ethyl)-6-fluoro-3-(2-fluorophenyl)-4H-chromen-4-one;
2-(1-(6-Amino-9H-purin-9-yl)ethyl)-3-(3,5-difluorophenyl)-4H-chromen-4-one;
2-(1-(6-amino-9H-purin-9-yl)ethyl)-6-fluoro-3-(3-fluorophenyl)-4H-chromen-4-one;
2-(1-(4-amino-3-(3-methoxyphenyl)-1H-pyrazolo[3,4-d]pyrimidin-1-yl)ethyl)-3-(3-fluorophenyl)-4H-chromen-4-one;
2-(1-(4-amino-3-(3-hydroxyphenyl)-1 H-pyrazolo [3,4-d] pyrimidin-1-yl) ethyl)-3-(3-fluorophenyl)-4H-chromen-4-one;
2-((4-Amino-3-(3-methoxyphenyl)-1H-pyrazolo [3,4-d] pyrimidin-1-yl) methyl)-3-phenyl-4H-chromen-4-one;
2-((4-Amino-3-(3-hydroxyphenyl)-1H-pyrazolo [3,4-d] pyrimidin-1-yl) methyl)-3-phenyl-4H-chromen-4-one;
2-((4-amino-3-(3-methoxyphenyl)-1H-pyrazolo [3,4-d] pyrimidin-1-yl) methyl)-3-(3-fluorophenyl)-4H-chromen-4-one;
2-((4-amino-3-(3-hydroxyphenyl)-1H-pyrazolo [3,4-d] pyrimidin-1-yl) methyl)-3-(3-fluorophenyl)-4H-chromen-4-one;
(R)-2-(1-(9H-purin-6-ylamino) ethyl)-3-(3-fluorophenyl)-4H-chromen-4-one;
(S)-2-(1-(9H-purin-6-ylamino) ethyl)-6-fluoro-3-phenyl-4H-chromen-4-one;
2-((4-Amino-3-iodo-1H-pyrazolo [3,4-d] pyrimidin-1-yl) methyl)-3-phenyl-4H-chromen-4-one;
2-(1-(4-amino-3-iodo-1H-pyrazolo [3,4-d] pyrimidin-1-yl) ethyl)-3-phenyl-4H-chromen-4-one;
2-(1-(4-amino-3-iodo-1H-pyrazolo [3,4-d] pyrimidin-1-yl) ethyl)-3-(3-fluorophenyl)-4H-chromen-4-one;
2-((4-amino-3-iodo-1H-pyrazolo [3,4-d] pyrimidin-1-yl) methyl)-6-fluoro-3-phenyl-4H-chromen-4-one;
2-(1-(4-amino-3-iodo-1H-pyrazolo [3,4-d] pyrimidin-1-yl) ethyl)-6-fluoro-3-phenyl-4H-chromen-4-one;
2-(1-(4-amino-3-iodo-1H-pyrazolo [3,4-d] pyrimidin-1-yl) ethyl)-6-fluoro-3-(3-fluorophenyl)-4H-chromen-4-one;
2-(1-(4-ammo-3-iodo-1H-pyrazolo [3,4-d] pyrimidin-1-yl) propyl)-3-(3-fluorophenyl)-4H-chromen-4-one;
2-((4-amino-3-(pyridin-3-yl)-1H-pyrazolo [3,4-d] pyrimidin-1-yl) methyl)-3-phenyl-4H-chromen-4-one;
2-((4-amino-3-(3-hydroxyprop-1-ynyl)-1H-pyrazolo [3,4-d] pyrimidin-1-yl) methyl)-3-phenyl-4H-chromen-4-one;
2-((4-amino-3-(1H-pyrazol-4-yl)-1H-pyrazolo [3,4-d] pyrimidin-1-yl) methyl)-3-phenyl-4H-chromen-4-one;
2-((4-amino-3-(3-(hydroxymethyl) phenyl)-1H-pyrazolo [3,4-d] pyrimidin-1-yl) methyl)-3-phenyl-4H-chromen-4-one;
2-((4-amino-3-(1H-indazol-4-yl)-1H-pyrazolo [3,4-d] pyrimidin-1-yl) methyl)-3-phenyl-4H-chromen-4-one;
2-((4-amino-3-(3-fluorophenyl)-1H-pyrazolo [3,4-d] pyrimidin-1-yl) methyl)-3-phenyl-4H-chromen-4-one
2-((4-amino-3-(3-hydroxypropyl)-1H-pyrazolo [3,4-d] pyrimidin-1-yl) methyl)-3-phenyl-4H-chromen-4-one;
N-(3-(4-amino-1-((4-oxo-3-phenyl-4H-chromen-2-yl) methyl)-1H-pyrazolo [3,4-d] pyrimidin-3-yl) phenyl) acetamide;
2-((4-amino-3-(3-fluoro-5-methoxyphenyl)-1 H-pyrazolo [3,4-d] pyrimidin-1-yl) methyl)-3-phenyl-4H-chromen-4-one;
2-((4-amino-3-(3-fluoro-5-hydroxyphenyl)-1H-pyrazolo [3,4-d] pyrimidin-1-yl) methyl)-3-phenyl-4H-chromen-4-one;
2-((4-amino-3-(3-fluoro-5-methoxyphenyl)-1 H-pyrazolo [3,4-d] pyrimidin-1-yl) methyl)-3-(3-fluorophenyl)-4H-chromen-4-one;
2-((4-amino-3-(3-fluoro-5-hydroxyphenyl)-1H-pyrazolo [3,4-d] pyrimidin-1-yl) methyl)-3-(3-fluorophenyl)-4H-chromen-4-one;
2-(1-(4-amino-3-(1 H-pyrazol-4-yl)-1 H-pyrazolo [3,4-d] pyrimidin-1-yl) ethyl)-3-phenyl-4H-chromen-4-one;
2-(1-(4-amino-3-(1H-indazol-6-yl)-1H-pyrazolo [3,4-d] pyrimidin-1-yl) ethyl)-3-phenyl-4H-chromen-4-one;
2-(1-(4-amino-3-(3-hydroxy-3-methylbut-1-ynyl)-1H-pyrazolo [3,4-d] pyrimidin-1-yl) ethyl)-3-phenyl-4H-chromen-4-one;
2-(1-(4-amino-3-(1 H-pyrazol-4-yl)-1 H-pyrazolo [3,4-d] pyrimidin-1-yl) ethyl)-3-(3-fluorophenyl)-4H-chromen-4-one;
(S)-2-(1-(9H-purin-6-ylamino) ethyl)-3-phenyl-4H-chromen-4-one;
(S)-2-(1-(9H-purin-6-ylamino) ethyl)-6-fluoro-3-(3-fluorophenyl)-4H-chromen-4-one;
2-((4-amino-3-(1H-indazol-6-yl)-1H-pyrazolo [3,4-d] pyrimidin-1-yl) methyl)-3-phenyl-4H-chromen-4-one;
2-(1-(4-amino-3-(3-fluoro-5-methoxyphenyl)-1H-pyrazolo [3,4-d] pyrimidin-1-yl) ethyl)-3-(3-fluorophenyl)-4H-chromen-4-one;
2-(1-(4-amino-3-(3-fluoro-5-hydroxyphenyl)-1H-pyrazolo [3,4-d] pyrimidin-1-yl) ethyl)-3-(3-fluorophenyl)-4H-chromen-4-one;
2-(1-(4-amino-3-(1H-indazol-4-yl)-1H-pyrazolo [3,4-d] pyrimidin-1-yl) ethyl)-3-(3-fluorophenyl)-4H-chromen-4-one;
2-(1-(4-amino-3-(3,5-dimethyl-1H-pyrazol-4-yl)-1H-pyrazolo [3,4-d] pyrimidin-1-yl) ethyl)-3-(3-fluorophenyl)-4H-chromen-4-one;
2-(1-(4-amino-3-(3-methyl-1H-indazol-6-yl)-1H-pyrazolo [3,4-d] pyrimidin-1-yl) ethyl)-3-(3-fluorophenyl)-4H-chromen-4-one;
2-(1-(4-amino-3-(1H-indazol-6-yl)-1H-pyrazolo [3,4-d] pyrimidin-1-yl) ethyl)-3-(3-fluorophenyl)-4H-chromen-4-one;
2-(1-(4-amino-3-(2-(hydroxymethyl) phenyl)-1H-pyrazolo [3,4-d] pyrimidin-1-yl) ethyl)-3-(3-fluorophenyl)-4H-chromen-4-one;
2-(1-(4-amino-3-(4-fluoro-3-methoxyphenyl)-1H-pyrazolo [3,4-d] pyrimidin-1-yl) ethyl)-3-(3-fluorophenyl)-4H-chromen-4-one;
2-(1-(4-amino-3-(4-fluoro-3-hydroxyphenyl)-1H-pyrazolo [3,4-d] pyrimidin-1-yl) ethyl)-3-(3-fluorophenyl)-4H-chromen-4-one;
2-(1-(4-amino-3-(3-hydroxyprop-1-ynyl)-1H-pyrazolo [3,4-d] pyrimidin-1-yl) ethyl)-3-(3-fluorophenyl)-4H-chromen-4-one;
2-(1-(4-amino-3-(3-fluoro-4-methoxyphenyl)-1H-pyrazolo [3,4-d] pyrimidin-1-yl) ethyl)-3-(3-fluorophenyl)-4H-chromen-4-one;
2-(1-(4-amino-3-(3-fluoro-4-hydroxyphenyl)-1H-pyrazolo [3,4-d] pyrimidin-1-yl) ethyl)-3-(3-fluorophenyl)-4H-chromen-4-one;
2-(1-(4-amino-3-(3-chloro-5-methoxyphenyl)-1H-pyrazolo [3,4-d] pyrimidin-1-yl) ethyl)-3-(3-fluorophenyl)-4H-chromen-4-one;
2-(1-(4-amino-3-(3-chloro-5-hydroxyphenyl)-1H-pyrazolo [3,4-d] pyrimidin-1-yl) ethyl)-3-(3-fluorophenyl)-4H-chromen-4-one;
2-(1-(4-amino-3-(3-(trifluoromethoxy) phenyl)-1H-pyrazolo [3,4-d] pyrimidin-1-yl) ethyl)-
3-(3-fluorophenyl)-4H-chromen-4-one;
2-(1-(4-amino-3-(4-methoxyphenyl)-1H-pyrazolo [3,4-d] pyrimidin-1-yl) ethyl)-3-(3-fluorophenyl)-4H-chromen-4-one;
2-(1-(4-amino-3-(4-hydroxyphenyl)-1H-pyrazolo [3,4-d] pyrimidin-1-yl) ethyl)-3-(3-fluorophenyl)-4H-chromen-4-one;
2-((6-amino-9H-purin-9-yl) methyl)-3-(3-fluorophenyl)-4H-chromen-4-one;
2-(1-(4-amino-3-(4-fluoro-2-methoxyphenyl)-1H-pyrazolo [3,4-d] pyrimidin-1-yl) ethyl)-3-(3-fluorophenyl)-4H-chromen-4-one;
2-(1-(4-amino-3-(4-fluoro-2-hydroxyphenyl)-1H-pyrazolo [3,4-d] pyrimidin-1-yl) ethyl)-3-(3-fluorophenyl)-4H-chromen-4-one;
2-((4-amino-3-(3-aminophenyl)-1H-pyrazolo [3,4-d] pyrimidin-1-yl) methyl)-3-phenyl-4H-chromen-4-one;
2-((4-amino-3-(3-methyl-1H-indazol-6-yl)-1H-pyrazolo [3,4-d] pyrimidin-1-yl) methyl)-3-phenyl-4H-chromen-4-one;
2-(1-(4-amino-3-(2-aminopyrimidin-5-yl)-1H-pyrazolo [3,4-d] pyrimidin-1-yl) ethyl)-3-(3-fluorophenyl)-4H-chromen-4-one;
2-(1-(4-amino-3-(1H-indol-6-yl)-1H-pyrazolo [3,4-d] pyrimidin-1-yl) ethyl)-3-(3-fluorophenyl)-4H-chromen-4-one;
2-(1-(4-amino-3-(4-chloro-3-methoxyphenyl)-1H-pyrazolo [3,4-d] pyrimidin-1-yl) ethyl)-3-(3-fluorophenyl)-4H-chromen-4-one;
2-(1-(4-amino-3-(4-chloro-3-hydroxyphenyl)-1H-pyrazolo [3,4-d] pyrimidin-1-yl) ethyl)-3-(3-fluorophenyl)-4H-chromen-4-one;
2-(1-(4-amino-3-(2-chloro-5-methoxyp enyl)-1H-pyrazolo [3,4-d] pyrimidin-1-yl) ethyl)-3-(3-fluorophenyl)-4H-chromen-4-one;
2-(1-(4-amino-3-(2-chloro-5-hydroxyphenyl)-1H-pyrazolo [3,4-d] pyrimidin-1-yl) ethyl)-3-(3-fluorophenyl)-4H-chromen-4-one;
2-(1-(4-amino-3-(3,4-dimethoxyphenyl)-1H-pyrazolo [3,4-d] pyrimidin-1-yl) ethyl)-3-(3-fluorophenyl)-4H-chromen-4-one;
2-(1-(4-amino-3-(3,4-dihydroxyphenyl)-1 H-pyrazolo [3,4-d] pyrimidin-1-yl) ethyl)-3-(3-fluorophenyl)-4H-chromen-4-one;
2-((4-amino-3-(3-methyl-1 H-indazol-6-yl)-1 H-pyrazolo [3,4-d] pyrimidin-1-yl) methyl)-3-(3-fluorophenyl)-4H-chromen-4-one;
2-(1-(4-amino-3-(1H-indol-5-yl)-1H-pyrazolo [3,4-d] pyrimidin-1-yl) ethyl)-3-(3-fluorophenyl)-4H-chromen-4-one;
2-(1-(4-Amino-3-(3-methyl-1H-indol-5-yl)-1H-pyrazolo [3,4-d] pyrimidin-1-yl) ethyl)-3-(3-fluorophenyl)-4H-chromen-4-one;
tert-butyl-(5-(4-amino-1-(1-(3-(3-fluorophenyl)-4-oxo-4H-chromen-2-yl) ethyl)-1 H-pyrazolo [3,4-d] pyrimidin-3-yl) thiophen-2-yl) methylcarbamate
2-(1-(4-amino-3-(5-(aminomethyl) thiophen-2-yl)-1H-pyrazolo [3,4-d] pyrimidin-1-yl) ethyl)-3-(3-fluorophenyl)-4H-chromen-4-one;
2-((4-amino-3-(3-methyl-1 H-indazol-6-yl)-1 H-pyrazolo [3,4-d] pyrimidin-1-yl) methyl)-6-fluoro-3-phenyl-4H-chromen-4-one;
2-(1-(4-amino-3-(3-methyl-1 H-indazol-6-yl)-1 H-pyrazolo [3,4-d] pyrimidin-1-yl) ethyl)-3-phenyl-4H-chromen-4-one;
2-(1-(4-amino-3-(3-methyl-1 H-indazol-6-yl)-1 H-pyrazolo [3,4-d] pyrimidin-1-yl) ethyl)-6-fluoro-3-phenyl-4H-chromen-4-one;
2-(1-(4-amino-3-(3-methyl-1H-indazol-5-yl)-1 H-pyrazolo [3,4-d] pyrimidin-1-yl) ethyl)-3-(3-fluorophenyl)-4H-chromen-4-one;
N-(4-(4-amino-1-(1-(3-(3-fluorophenyl)-4-oxo-4H-chromen-2-yl) ethyl)-1 H-pyrazolo [3,4-d] pyrimidin-3-yl) phenyl) acetamide;
2-(1-(4-amino-3-(4-aminophenyl)-1 H-pyrazolo [3,4-d] pyrimidin-1-yl) ethyl)-3-(3-fluorophenyl)-4H-chromen-4-one;
2-(1-(4-amino-3-(3-methyl-1H-indazol-6-yl)-1H-pyrazolo [3,4-d] pyrimidin-1-yl) ethyl)-6-fluoro-3-(3-fluorophenyl)-4H-chromen-4-one;
2-(1-(4-amino-3-(2,3-dihydrobenzofuran-5-yl)-1 H-pyrazolo [3,4-d] pyrimidin-1-yl) ethyl)-3-(3-fluorophenyl)-4H-chromen-4-one;
2-(1-(4-amino-3-(3-ethyl-1H-indazol-6-yl)-1H-pyrazolo [3,4-d] pyrimidin-1-yl) ethyl)-3-(3-fluorophenyl)-4H-chromen-4-one;
2-(1-(4-amino-3-(3-methyl-1 H-indol-6-yl)-1 H-pyrazolo [3,4-d] pyrimidin-1-yl) ethyl)-3-(3-fluorophenyl)-4H-chromen-4-one;
2-(1-(4-amino-3-(2-methoxypyrimidin-5-yl)-1 H-pyrazolo [3,4-d] pyrimidin-1-yl) ethyl)-3-(3-fluorophenyl)-4H-chromen-4-one;
4-(4-amino-1-(1-(3-(3-fluorophenyl)-4-oxo-4H-chromen-2-yl) ethyl)-1 H-pyrazolo [3,4-d] pyrimidin-3-yl) thiophene-2-carbaldehyde;
2-(1-(4-amino-3-(5-(hydroxymethyl) thiophen-3-yl)-1H-pyrazolo [3,4-d] pyrimidin-1-yl) ethyl)-3-(3-fluorophenyl)-4H-chromen-4-one;
2-(1-(4-amino-3-(2-methyl-1H-benzo[d]imidazol-5-yl)-1H-pyrazolo [3,4-d] pyrimidin-1-yl) ethyl)-3-(3-fluorophenyl)-4H-chromen-4-one;
2-(1-(4-amino-3-(3-methyl-1 H-indazol-6-yl)-1 H-pyrazolo [3,4-d] pyrimidin-1-yl) propyl)-3-(3-fluorophenyl)-4H-chromen-4-one;
2-(1-(4-amino-3-(3-methyl-1H-indol-6-yl)-1H-pyrazolo [3,4-d] pyrimidin-1-yl) ethyl)-3-(3-fluorophenyl)-4H-chromen-4-one;
2-((6-amino-9H-purin-9-yl) methyl)-6-fluoro-3-phenyl-4H-chromen-4-one;
2-((6-amino-9H-purin-9-yl) methyl)-6-fluoro-3-(3-fluorophenyl)-4H-chromen-4-one;
2-((4-amino-3-(3-fluoro-5-methoxyphenyl)-1H-pyrazolo [3,4-d] pyrimidin-1-yl) methyl)-6-fluoro-3-(3-fluorophenyl)-4H-chromen-4-one;
2-((4-amino-3-(3-fluoro-5-hydroxyphenyl)-1H-pyrazolo [3,4-d] pyrimidin-1-yl) methyl)-6-fluoro-3-(3-fluorophenyl)-4H-chromen-4-one;
2-(1-(4-amino-3-(3-methoxyphenyl)-1H-pyrazolo [3,4-d] pyrimidin-1-yl) ethyl)-6-fluoro-3-(3-fluorophenyl)-4H-chromen-4-one;
2-(1-(4-amino-3-(3-hydroxyphenyl)-1H-pyrazolo [3,4-d] pyrimidin-1-yl) ethyl)-6-fluoro-3-(3-fluorophenyl)-4H-chromen-4-one;
2-((9H-purin-6-ylamino) methyl)-6-fluoro-3-(3-fluorophenyl)-4H-chromen-4-one;
2-((9H-purin-6-ylamino) methyl)-6-fluoro-3-phenyl-4H-chromen-4-one;
(R)-2-(1-(9H-purin-6-ylamino) ethyl)-6-fluoro-3-(3-fluorophenyl)-4H-chromen-4-one;
2-((4-amino-3-(1H-pyrazol-4-yl)-1H-pyrazolo [3,4-d] pyrimidin-1-yl) methyl)-6-fluoro-3-phenyl-4H-chromen-4-one;
2-(1-(4-amino-3-(3,5-difluoro-4-methoxyphenyl)-1H-pyrazolo [3,4-d] pyrimidin-1-yl) ethyl)-3-(3-fluorophenyl)-4H-chromen-4-one;
2-(1-(4-amino-3-(3,5-difluoro-4-hydroxyphenyl)-1H-pyrazolo [3,4-d] pyrimidin-1-yl) ethyl)-3-(3-fluorophenyl)-4H-chromen-4-one;
2-((4-amino-3-(3,5-difluoro-4-methoxyphenyl)-1H-pyrazolo [3,4-d] pyrimidin-1-yl) methyl)-6-fluoro-3-(3-fluorophenyl)-4H-chromen-4-one;
2-((4-amino-3-(3,5-difluoro-4-hydroxyphenyl)-1H-pyrazolo [3,4-d] pyrimidin-1-yl) methyl)-6-fluoro-3-(3-fluorophenyl)-4H-chromen-4-one;
(+)-2-(1-(4-amino-3-(3-methyl-1H-indazol-6-yl)-1H-pyrazolo [3,4-d] pyrimidin-1-yl) ethyl)-3-(3-fluorophenyl)-4H-chromen-4-one;
(−)-2-(1-(4-amino-3-(3-methyl-1H-indazol-6-yl)-1H-pyrazolo [3,4-d] pyrimidin-1-yl) ethyl)-3-(3-fluorophenyl)-4H-chromen-4-one;
2-(1-(4-amino-3-(3,5-dimethoxyphenyl)-1H-pyrazolo [3,4-d] pyrimidin-1-yl) ethyl)-3-(3-fluorophenyl)-4H-chromen-4-one;
2-(1-(4-amino-3-(4-methoxy-3,5-dimethylphenyl)-1H-pyrazolo [3,4-d] pyrimidin-1-yl) ethyl)-3-(3-fluorophenyl)-4H-chromen-4-one;
2-(1-(4-amino-3-(2-fluoro-5-isopropoxyphenyl)-1H-pyrazolo [3,4-d] pyrimidin-1-yl) ethyl)-
3-(3-fluorophenyl)-4H-chromen-4-one;
2-(1-(4-amino-3-(2,3-dihydrobenzo[b] [1,4] dioxin-6-yl)-1H-pyrazolo [3,4-d] pyrimidin-1-yl) ethyl)-3-(3-fluorophenyl)-4H-chromen-4-one;
2-(1-(4-amino-3-(1-benzyl-1H-pyrazol-4-yl)-1H-pyrazolo [3,4-d] pyrimidin-1-yl) ethyl)-3-(3-fluorophenyl)-4H-chromen-4-one;
2-(1-(4-amino-3-(2-methylpyridin-4-yl)-1H-pyrazolo [3,4-d] pyrimidin-1-yl) ethyl)-3-(3-fluorophenyl)-4H-chromen-4-one;
2-(1-(4-amino-3-(3,4-dihydro-2H-benzo[b] [1,4] dioxepin-7-yl)-1H-pyrazolo [3,4-d] pyrimidin-1-yl) ethyl)-3-(3-fluorophenyl)-4H-chromen-4-one;
2-(1-(4-amino-3-(6-morpholinopyridin-3-yl)-1H-pyrazolo [3,4-d] pyrimidin-1-yl) ethyl)-3-(3-fluorophenyl)-4H-chromen-4-one;
2-(1-(4-amino-3-(dibenzo [b, d] furan-4-yl)-1H-pyrazolo [3,4-d] pyrimidin-1-yl) ethyl)-3-(3-fluorophenyl)-4H-chromen-4-one;
2-(1-(4-amino-3-(4-phenoxyphenyl)-1H-pyrazolo [3,4-d] pyrimidin-1-yl) ethyl)-3-(3-fluorophenyl)-4H-chromen-4-one;
2-(1-(4-amino-3-(4-(benzyloxy)-3-chlorophenyl)-1H-pyrazolo [3,4-d] pyrimidin-1-yl) ethyl)-3-(3-fluorophenyl)-4H-chromen-4-one;
2-(1-(4-amino-3-(3-chloro-4-isopropoxyphenyl)-1H-pyrazolo [3,4-d] pyrimidin-1-yl) ethyl)-
3-(3-fluorophenyl)-4H-chromen-4-one;
2-(1-(4-amino-3-(3-(dimethylamino) phenyl)-1H-pyrazolo [3,4-d] pyrimidin-1-yl) ethyl)-3-(3-fluorophenyl)-4H-chromen-4-one;
2-(1-(4-amino-3-(4-ethoxy-3-fluorophenyl)-1H-pyrazolo [3,4-d] pyrimidin-1-yl) ethyl)-3-(3-fluorophenyl)-4H-chromen-4-one;
2-(1-(4-amino-3-(4-isopropoxyphenyl)-1H-pyrazolo [3,4-d] pyrimidin-1-yl) ethyl)-3-(3-fluorophenyl)-4H-chromen-4-one;
2-(1-(4-amino-3-(4-(trifluoromethoxy) phenyl)-1H-pyrazolo [3,4-d] pyrimidin-1-yl) ethyl)-
3-(3-fluorophenyl)-4H-chromen-4-one;
2-(1-(3-(4-acetylphenyl)-4-amino-1H-pyrazolo [3,4-d] pyrimidin-1-yl) ethyl)-3-(3-fluorophenyl)-4H-chromen-4-one;
2-(1-(4-amino-3-(4-(benzyloxy) phenyl)-1H-pyrazolo [3,4-d] pyrimidin-1-yl) ethyl)-3-(3-fluorophenyl)-4H-chromen-4-one;
2-(1-(4-amino-3-(4-(dimethylamino) phenyl)-1H-pyrazolo [3,4-d] pyrimidin-1-yl) ethyl)-3-(3-fluorophenyl)-4H-chromen-4-one;
2-(1-(4-amino-3-(4-(methylsulfonyl) phenyl)-1H-pyrazolo [3,4-d] pyrimidin-1-yl) ethyl)-3-(3-fluorophenyl)-4H-chromen-4-one;
2-(1-(4-amino-3-(3-ethoxyphenyl)-1 H-pyrazolo [3,4-d] pyrimidin-1-yl) ethyl)-3-(3-fluorophenyl)-4H-chromen-4-one;
2-(1-(4-amino-3-(benzo[b]thiophen-2-yl)-1H-pyrazolo [3,4-d] pyrimidin-1-yl) ethyl)-3-(3-fluorophenyl)-4H-chromen-4-one;
2-(1-(4-amino-3-(5-chlorothiophen-2-yl)-1H-pyrazolo [3,4-d] pyrimidin-1-yl) ethyl)-3-(3-fluorophenyl)-4H-chromen-4-one;
2-(1-(4-amino-3-(3,5-dimethylisoxazol-4-yl)-1H-pyrazolo [3,4-d] pyrimidin-1-yl) ethyl)-3-(3-fluorophenyl)-4H-chromen-4-one;
2-(1-(4-amino-3-(3-propoxyphenyl)-1H-pyrazolo [3,4-d] pyrimidin-1-yl) ethyl)-3-(3-fluorophenyI)-4H-chromen-4-one;
2-(1-(4-amino-3-(furan-2-yl)-1H-pyrazolo [3,4-d] pyrimidin-1-yl) ethyl)-3-(3-fluorophenyl)-4H-chromen-4-one;
2-(1-(4-amino-3-(4-ethoxyphenyl)-1H-pyrazolo [3,4-d] pyrimidin-1-yl) ethyl)-3-(3-fluorophenyl)-4H-chromen-4-one;
2-(1-(4-amino-3-(3-chloro-4-methoxyphenyl)-1H-pyrazolo [3,4-d] pyrimidin-1-yl) ethyl)-3-(3-fluorophenyl)-4H-chromen-4-one;
2-(1-(4-amino-3-(3-fluoro-4-isopropoxyphenyl)-1H-pyrazolo [3,4-d] pyrimidin-1-yl) ethyl)-
3-(3-fluorophenyl)-4H-chromen-4-one;
2-(1-(4-amino-3-(6-fluoropyridin-3-yl)-1H-pyrazolo [3,4-d] pyrimidin-1-yl) ethyl)-3-(3-fluorophenyl)-4H-chromen-4-one;
2-(1-(4-amino-3-(pyrimidin-5-yl)-1H-pyrazolo [3,4-d] pyrimidin-1-yl) ethyl)-3-(3-fluorophenyl)-4H-chromen-4-one;
2-(1-(4-amino-3-(3-(methoxymethyl) phenyl)-1H-pyrazolo [3,4-d] pyrimidin-1-yl) ethyl)-3-(3-fluorophenyl)-4H-chromen-4-one;
2-(1-(4-amino-3-(6-hydroxynaphthalen-2-yl)-1H-pyrazolo [3,4-d] pyrimidin-1-yl) ethyl)-3-(3-fluorophenyl)-4H-chromen-4-one;
2-(1-(4-amino-3-(3-isopropoxyphenyl)-1H-pyrazolo [3,4-d] pyrimidin-1-yl) ethyl)-3-(3-fluorophenyl)-4H-chromen-4-one;
2-(1-(4-amino-3-(1-methyl-1H-pyrazol-4-yl)-1H-pyrazolo [3,4-d] pyrimidin-1-yl) ethyl)-3-(3-fluorophenyl)-4H-chromen-4-one;
6-Fluoro-3-(3-fluorophenyl)-2-(1-(4-methoxyphenylamino) ethyl)-4H-chromen-4-one;
2-(1-(4-Chloro-7H-pyrrolo [2,3-d] pyrimidin-7-yl) emyl)-3-(3-fluorophenyl)-4H-chromen-4-one; 2-(1-(4-Chloro-1H-pyrazolo [3,4-b] pyridin-1-yl) ethyl)-3-(3-fluorophenyl)-4H-chromen-4-one;
2-(1-(4-Chloro-1H-pyrazolo [3,4-d] pyrimidin-1-yl) ethyl)-3-(3-fluorophenyl)-4H-chromen-4-one;
2-(1-(4-Chloro-5H-pyrrolo [3,2-d] pyrimidin-5-yl) ethyl)-3-(3-fluorophenyl)-4H-chromen-4-one;
2-(1-(4-amino-3-(1,3-dimethyl-1H-indazol-6-yl)-1H-pyrazolo [3,4-d] pyrimidin-1-yl) ethyl)-3-(3-fluorophenyl)-4H-chromen-4-one;
2-(1-(4-amino-3-(2,3-dimethyl-2H-indazol-6-yl)-1H-pyrazolo [3,4-d] pyrimidin-1-yl) ethyl)-3-(3-fluorophenyl)-4H-chromen-4-one;
2-(1-(4-amino-3-(6-methoxynaphthalen-2-yl)-1 H-pyrazolo [3,4-d] pyrimidin-1-yl) ethyl)-3-(3-fluorophenyl)-4H-chromen-4-one;
2-(1-(4-amino-3-(benzo[b]thiophen-3-yl)-1H-pyrazolo [3,4-d] pyrimidin-1-yl) ethyl)-3-(3-fluorophenyl)-4H-chromen-4-one;
2-(1-(4-amino-3-(2,4-dimethoxypyrimidin-5-yl)-1 H-pyrazolo [3,4-d] pyrimidin-1-yl) ethyl)-
3-(3-fluorophenyl)-4H-chromen-4-one;
2-(1-(4-amino-3-(6-ethoxynaphthalen-2-yl)-1 H-pyrazolo [3,4-d] pyrimidin-1-yl) ethyl)-
3-(3-fluorophenyl)-4H-chromen-4-one;
3-(4-amino-1-(1-(3-(3-fluorophenyl)-4-oxo-4H-chromen-2-yl) ethyl)-1 H-pyrazolo [3,4-d] pyrimidin-3-yl)-N-cyclopropylbenzamide;
2-(1-(4-amino-3-(3-(morpholine-4-carbonyl) phenyl)-1 H-pyrazolo [3,4-d] pyrimidin-1-yl) ethyl)-3-(3-fluorophenyl)-4H-chromen-4-one;
2-(1-(4-amino-3-(3-(difluoromethoxy) phenyl)-1 H-pyrazolo [3,4-d] pyrimidin-1-yl) ethyl)-3-(3-fluorophenyl)-4H-chromen-4-one;
5-(4-amino-1-(1-(3-(3-fluorophenyl)-4-oxo-4H-chromen-2-yl) ethyl)-1 H-pyrazolo [3,4-d] pyrimidin-3-yl) furan-2-carbaldehyde and pharmaceutically acceptable salts thereof;
   or their enantiomers, mixtures of enantiomers, mixtures of two or more diastereoisomers, isotopic variants, pharmaceutically acceptable salts, solvates, hydrates or pro-drug.

In certain embodiment, wherein said PI3K inhibitor comprise: 2-(1-(9H-purin-6-ylamino) propyl group)-3-(3-fluorophenyl)-4H-chromen-4-one or its enantiomers, mixtures of enantiomers, mixtures of two or more diastereoisomers, isotopic variants, pharmaceutically acceptable salts, solvates, hydrates or prodrugs.

In certain embodiment, wherein said PI3K inhibitor comprise: (S)-2-(1-(9H-purin-6-ylamino) propyl group)-3-(3-fluorophenyl)-4H-chromen-4-one or its pharmaceutically acceptable salts, solvates, hydrates or prodrugs.

In certain embodiment, wherein said pharmaceutical composition comprises a PD-L1/TGFβ dual inhibitor and a PI3K inhibitor.

In certain embodiment, wherein the PD-L1/TGFβ dual inhibitor comprises an anti-PD-L1 antibody or an antigen-binding fragment thereof and TGFβRII or a functionally active fragment thereof.

In certain embodiment, wherein the PD-L1/TGFβ dual inhibitor comprises two polypeptides comprising an amino acid sequence selected from the group consisting of: (a) the amino acid sequence shown in SEQ ID NO:7; (b) an amino acid sequence having at least 85%, 90%, 95% or 99% sequence identity with the amino acid sequence shown in SEQ ID NO: 7; (c) an amino acid sequence having one or more differences from the amino acid sequence shown in SEQ ID NO:7 obtained by addition, elimination or substitution reaction.

In certain embodiment, wherein the PI3K inhibitor is a dual PI3K δ/γ inhibitor.

In In certain embodiment, wherein the PI3K inhibitor is (S)-2-(1-(9H-purin-6-ylamino) propyl group)-3-(3-fluorophenyl)-4H-chromen-4-one or its pharmaceutically acceptable salts, solvates, hydrates or prodrugs.

In certain embodiment, wherein the said PD-L1/TGFβ dual inhibitor and the PI3K inhibitor are not intermixed in the pharmaceutical combination.

In certain embodiment, wherein the second therapeutic agent and the PI3K inhibitor are each independently present in separate containers.

In another aspect, the present application provides a pharmaceutical composition, comprising the aforementioned PI3K inhibitor and a second therapeutic agent, and optionally one or more pharmaceutically acceptable carriers or excipients.

In certain embodiment, wherein the second therapeutic agent is the aforementioned PD-L1/TGFβ dual inhibitor.

In certain embodiment, the PI3K inhibitor is the aforementioned PI3Kδ/γ dual inhibitor. In certain embodiment, wherein said second therapeutic agent and said PI3K inhibitor are present in a single or separate dosage form.

In another aspect, the present application provides a method for treating and/or preventing tumors, which comprises administering to a subject in need: an effective amount of the aforementioned pharmaceutical combination or the aforementioned pharmaceutical composition.

In certain embodiment, the method includes administering to a subject in need: an effective amount of the aforementioned PI3K inhibitor and the aforementioned immune checkpoint inhibitor.

In certain embodiment, the method comprises administering to a subject in need: an effective amount of the aforementioned PI3K inhibitor and the aforementioned TGFβ inhibitor. In certain embodiment, the method includes administering to a subject in need: an effective amount of the aforementioned PI3K inhibitor, the aforementioned TGFβ inhibitor, and the aforementioned immune checkpoint inhibitor.

In certain embodiment, the method includes administering to a subject in need: an effective amount of the aforementioned PI3K inhibitor and the aforementioned PD-L1/TGFβ dual inhibitor.

In certain embodiment, the PI3K inhibitor and the PD-L1/TGFβ dual inhibitor are administered simultaneously.

In a certain embodiment, the PI3K inhibitor is administered after the PD-L1/TGFβ dual inhibitor.

In a certain embodiment, the PI3K inhibitor is administered before the PD-L1/TGFβ dual inhibitor.

In certain embodiment, the present invention also includes administering an effective amount of the aforementioned small molecule TGFβ inhibitor to a subject in need.

In certain embodiment, wherein the tumor includes solid tumors and non-solid tumors.

In In certain embodiment, wherein the tumor comprises a tumor with abnormal expression of PI3K.

In certain embodiment, wherein the tumor comprises a tumor with abnormal expression of PI3K.

In certain embodiment, wherein the tumor comprises a tumor with abnormal expression of PD-L1.

In certain embodiment, wherein the tumor comprises a tumor with abnormal expression of TGFβ.

In certain embodiment, the tumor comprises gastrointestinal tumors, melanoma, or lymphoma.

In certain embodiment, wherein said tumor has delayed resistance to said PI3K inhibitor.

In In certain embodiment, wherein the tumor in the subject has prolonged remission.

In In certain embodiment, wherein the subject experiences a complete remission of the tumor.

In In certain embodiment, wherein the level of minimal residual disease (MRD) is reduced.

In In another aspect, the present application provides the use of the aforementioned pharmaceutical combination or the aforementioned pharmaceutical composition in the preparation of a medicament for treating and/or preventing tumors.

For example, the use of the aforementioned PI3K inhibitors and the aforementioned immune checkpoint inhibitors in the manufacture of drugs for treating and/or preventing tumors. For example, the use of the aforementioned PI3K inhibitor and the aforementioned TGFβ inhibitor in the manufacture of medicaments for treating and/or preventing tumors. For example, the use of the aforementioned PI3K inhibitors, the aforementioned immune checkpoint inhibitors and the aforementioned TGFβ inhibitors in the manufacture of drugs for treating and/or preventing tumors.

In another aspect, the present application provides the aforementioned pharmaceutical combination or the aforementioned pharmaceutical composition for treating and/or preventing tumors.

In another aspect, the present application provides a method for inhibiting tumor growth, the said method comprising exposing the tumor to the aforementioned pharmaceutical combination or the aforementioned pharmaceutical composition.

In certain embodiment, the contacting is in an in vitro or ex vivo environment.

In In certain embodiment, the method comprises exposing the tumor to an aforementioned PI3K inhibitor and an aforementioned inhibitor of an immune checkpoint. In certain embodiment, the method comprises contacting the tumor with the aforementioned PI3K inhibitor and the aforementioned TGFβ inhibitor. In certain embodiment, the method comprises contacting the tumor with an aforementioned PI3K inhibitor, an aforementioned inhibitor of an immune checkpoint, and an aforementioned TGFβ inhibitor.

In another aspect, the present application provides a method of inhibiting tumor growth, the method comprising exposing the tumor to fresh PI3K and fresh PD-L1/TGFβ dual inhibitor.

In another aspect, the present application provides a kit, which includes: (1) a first container, and the aforementioned PI3K inhibitor located in the first container; (2) a second container, and the aforementioned PD-L1/TGFβ dual inhibitors in the second container.

In another aspect, the present application provides a kit, which includes: (1) a first container, and the aforementioned PI3K inhibitor located in the first container; (2) a second container, and the aforementioned immune checkpoint inhibitor or the aforementioned TGFβ inhibitor in the second container.

In certain embodiment, the kit further includes (3) a third container, and the aforementioned immune checkpoint inhibitor or the aforementioned TGFβ inhibitor located in the third container, the medicament in the third container different from the medicament in the second container.

In certain embodiment, the dosage form of the medicine in the kit is an oral dosage form or an injection dosage form.

In certain embodiment, the kit further includes instructions.

Those the skilled person in the field can easily perceive other aspects and advantages of the present application from the following detailed description. In the following detailed description, only exemplary embodiment of the present application are shown and described. As those skilled in the field will appreciate, the content of the present application enables those skilled in the art to make changes to the specific embodiment which are disclosed without departing from the spirit and scope of the invention to which this application relates. Correspondingly, the drawings and descriptions in the specification of the present application are only exemplary rather than restrictive.

### DESCRIPTION OF DRAWINGS

The particular features of the invention to which this application relates are set forth in the appended claims. The features and advantages of the invention to which this application relates can be better understood with reference to the exemplary embodiment described in detail hereinafter and the accompanying drawings. A brief description of the accompanying drawings is as follows:

FIGS. 1A-1F show the results of combined administration of CN401 (Tenalisib) (150 mg/kg) and CN202 (WBP1126) (5 mg/kg) in the A20 mouse B-cell lymphoma animal model.

FIGS. 2A-2F show the results of combined administration of CN401 (Tenalisib) (150 mg/kg) and CN202 (WBP1126) (15 mg/kg) in the A20 mouse B-cell lymphoma animal model.

FIGS. 3A-3B show the results of combined administration of CN401 (Tenalisib) (150 mg/kg) and CN202 (WBP1126) (5 mg/kg) (15 mg/kg) in the A20 mouse B-cell lymphoma animal model.

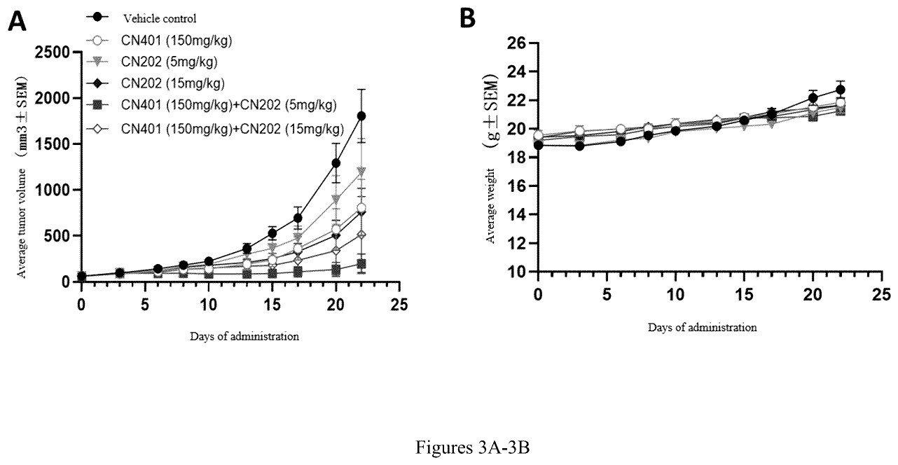

FIGS. 4A-4F show the results of combined administration of CN202 (WBP1126) (5 mg/kg) and CN401 (Tenalisib) (150 mg/kg) in the A20 mouse B-cell lymphoma animal model.

FIGS. 5A-5F show the results of combined administration of CN202 (WBP1126) (5 mg/kg) and Alpelisib (50 mg/kg) in the A20 mouse B-cell lymphoma animal model.

FIGS. 6A-6F show the results of combined administration of CN401 (Tenalisib) (150 mg/kg) and anti-PD-L1 antibody (Atezolizumab) (5 mg/kg) in the A20 mouse B-cell lymphoma animal model.

FIGS. 7A-7B show the effect of CN401 (Tenalisib) (150 mg/kg), CN202 (WBP1126) (15 mg/kg) and nab-paclitaxel (nab-pac) (10 mg/kg) on mouse EMT-60 (mouse breast cancer cells) tumor growth curve and body weight growth curve.

FIGS. 8A-8G show the tumor growth curve of mouse EMT-60 (mouse breast cancer cells) in different dosing groups.

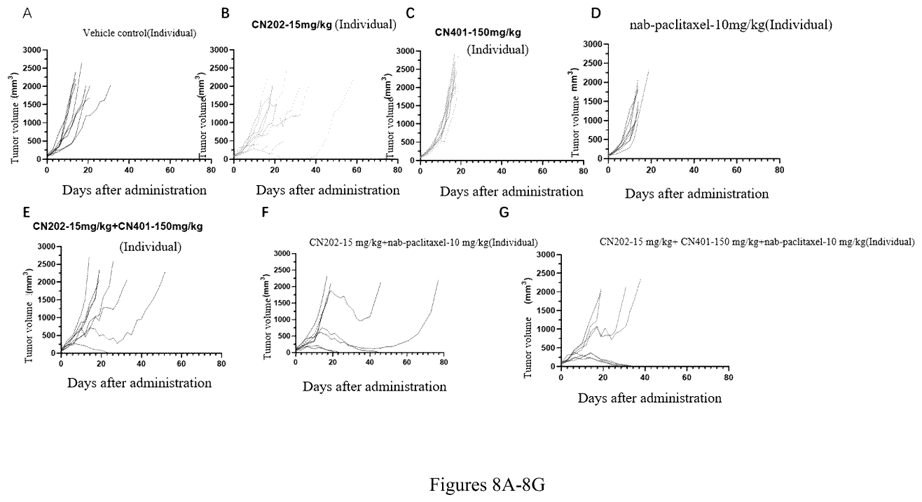

### EMBODIMENT

The implementation mode of the invention of the application is described below by specific examples, and those who are familiar with this technology can easily understand other advantages and effects of the invention of the application from the content disclosed in this description.

### Terms Definition

In the present application, “phosphoinositide 3-kinase (PI3K) inhibitor” or “PI3K inhibitor” generally refers to any inhibitor of PI3K. PI3Ks are members of a unique and conserved family of intracellular lipid kinases that phosphorylate the 3′-OH group on phosphatidylinositols or phosphoinositides. The PI3K family includes kinases with distinct substrate specificities, expression patterns, and regulatory patterns (see, e.g., Katso et al., 2001, Annu. Rev. Cell Dev. Biol. 17, 615-675; Foster, F. M. et al., 2003, J Cell Sci 116, 3037-3040). Class I PI3Ks (e.g., p110α, p110β, p110γ, and p110δ are normally activated by tyrosine kinases or G-protein-coupled receptors to generate PIPS, which binds downstream mediators such as those in the Akt/PDK1 pathway, mTOR, Tec family Kinases and Rho family GTPases. Class II PI3Ks (e.g. PI3K-C₂α, PI3K-C2β, PI3K-C2γ) and class III PI3Ks (e.g. Vps34) play a key role in intracellular trafficking by the synthesis of PI(3)P and PI(3,4)P2. Specific exemplary PI3K inhibitors are disclosed herein. For example, PI3K inhibitors inhibit PI3K-alpha, PI3K-beta, PI3K-gamma, and PI3K-delta subtypes or combinations thereof.

For example, the PI3K inhibitor can be Tenalisib, a dual PI3K delta/gamma inhibitor, 2-(1-(9H-purin-6-ylamino)propyl)-3-(3-fluorophenyl)-4H-chromene-4-ketone, its structural formula is as follows:

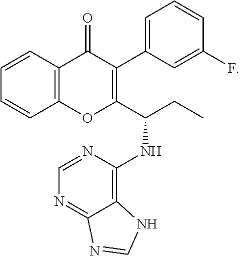 [SMILES: CC[C@H](Nc1ncnc2nc[nH]c12)c1oc2ccccc2c(=O)c1-c1cccc(F)c1]

For example, the PI3K inhibitor can be the PI3K inhibitor Alpelisib, (2S)-1-N-[4-methyl-5-[2-(1,1,1-trifluoro-2-methylpropan-2-yl)pyridin-4-yl]-1,3-thiazol-2-yl]pyrrolidine-1,2-dicarboxamide, its structural formula is as follows:

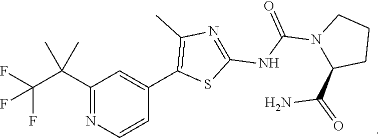 [SMILES: Cc1nc(NC(=O)N2CCC[C@H]2C(N)=O)sc1-c1ccnc(C(C)(C)C(F)(F)F)c1]

In certain embodiment, a PI3K inhibitor generally refers to a compound that inhibits one or more PI3K subtype with an IC50 of less than about 1000 nM, less than about 900 nM, less than about 800 nM, less than about 700 nM, less than about 600 nM, less than about 500 nM, Less than about 400 nM, less than about 300 nM, less than about 200 nM, less than about 100 nM, less than about 75 nM, less than about 50 nM, less than about 25 nM, less than about 20 nM, less than about 15 nM, less than about 10 nM, less than about 10 nM, less than about 5 nM, or less than about 1 nM.

The term “alkyl” refers to a straight or branched hydrocarbon chain radical consisting solely of carbon and hydrogen atoms, containing no unsaturation, having from one to eight carbon atoms, and which is attached to the rest of the molecule by a single bond, e.g., methyl, ethyl, n-propyl, 1-methylethyl (isopropyl), n-butyl, n-pentyl, and 1,1-dimethylethyl (t-butyl). The term “(C1-6)alkyl” refers to an alkyl group as defined above having up to 6 carbon atoms.

The term “alkenyl” refers to an aliphatic hydrocarbon group containing a carbon-carbon double bond and which may be a straight or branched or branched chain having about 2 to about 10 carbon atoms, e.g., ethenyl, 1-propenyl, 2-propenyl (allyl), iso-propenyl, 2-methyl-1-propenyl, 1-butenyl, and 2-butenyl. The term “(C2-6)alkenyl” refers to an alkenyl group as defined above having up to 6 carbon atoms.

The term “alkynyl” refers to a straight or branched chain hydrocarbyl radical having at least one carbon-carbon triple bond, and having in the range of 2 to up to 12 carbon atoms (with radicals having in the range of 2 to up to 10 carbon atoms presently being preferred) e.g., ethynyl, propynyl, and butnyl. The term “(C2-6) alkynyl” refers to an alkynyl group as defined above having up to 6 carbon atoms.

The term “alkoxy” denotes an alkyl, cycloalkyl, or cycloalkylalkyl group as defined above attached via an oxygen linkage to the rest of the molecule. The term “substituted alkoxy” refers to an alkoxy group where the alkyl constituent is substituted (i.e., -0-(substituted alkyl) wherein the term “substituted alkyl” is the same as defined above for “alkyl”. For example “alkoxy” refers to the group —O-alkyl, including from 1 to 8 carbon atoms of a straight, branched, cyclic configuration and combinations thereof attached to the parent structure through oxygen. Examples include methoxy, ethoxy, propoxy, isopropoxy, cyclopropyloxy, and cyclohexyloxy.

The term “cycloalkyl” denotes a non-aromatic mono or multicyclic ring system of about 3 to 12 carbon atoms such as cyclopropyl, cyclobutyl, cyclopentyl, and cyclohexyl. Examples of multicyclic cycloalkyl groups include perhydronaphthyl, adamantyl and norbornyl groups, bridged cyclic groups, and sprirobicyclic groups, e.g., sprio (4,4) non-2-yl. The term “(C3-8) cycloalkyl” refers to a cycloalkyl group as defined above having up to 8 carbon atoms.

The term “cycloalkylalkyl” refers to a cyclic ring-containing radical containing in the range of about 3 up to 8 carbon atoms directly attached to an alkyl group which are then attached to the main structure at any carbon from the alkyl group that results in the creation of a stable structure such as cyclopropylmethyl, cyclobutylethyl, and cyclopentylethyl.

The term “cycloalkenyl” refers to cyclic ring-containing radicals containing in the range of about 3 up to 8 carbon atoms with at least one carbon-carbon double bond such as cyclopropenyl, cyclobutenyl, and cyclopentenyl. The term “cycloalkenylalkyl” refers to a cycloalkenyl group directly attached to an alkyl group which are then attached to the main structure at any carbon from the alkyl group that results in the creation of a stable structure.

The term “aryl” refers to aromatic radicals having in the range of 6 up to 20 carbon atoms such as phenyl, naphthyl, tetrahydronaphthyl, indanyl, and biphenyl.

The term “arylalkyl” refers to an aryl group as defined above directly bonded to an alkyl group as defined above, e.g., —CH₂C₆H₅ and —C₂H₅C₆H₅.

The term “heterocyclic ring” refers to a non-aromatic 3 to 15 member ring radical which consists of carbon atoms and at least one heteroatom selected from nitrogen, phosphorus, oxygen and sulfur. For purposes of this invention, the heterocyclic ring radical may be a mono-, bi-, tri- or tetracyclic ring system, which may include fused, bridged or spiro ring systems, and the nitrogen, phosphorus, carbon, oxygen or sulfur atoms in the heterocyclic ring radical may be optionally oxidized to various oxidation states. In addition, the nitrogen atom may be optionally quatemized The heterocyclic ring radical may be attached to the main structure at any heteroatom or carbon atom that results in the creation of a stable structure.

The term “heterocyclyl” refers to a heterocylic ring radical as defined above. The heterocylcyl ring radical may be attached to the main structure at any heteroatom or carbon atom that results in the creation of a stable structure.

The term “heterocyclylalkyl” refers to a heterocylic ring radical as defined above directly bonded to an alkyl group. The heterocyclylalkyl radical may be attached to the main structure at carbon atom in the alkyl group that results in the creation of a stable structure. Examples of such heterocycloalkyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl.

The term “heteroaryl” refers to an optionally substituted 5 to 14 member aromatic ring having one or more heteroatoms selected from N, O, and S as ring atoms. The heteroaryl may be a mono-, bi- or tricyclic ring system. Examples of such “heterocyclic ring” or “heteroaryl” radicals include, but are not limited to, oxazolyl, thiazolyl, imidazolyl, pyrrolyl, furanyl, pyridinyl, pyrimidinyl, pyrazinyl, benzofuranyl, indolyl, benzothiazolyl, benzoxazolyl, carbazolyl, quinolyl , isoquinolyl, azetidinyl, acridinyl, benzodioxolyl, benzodioxanyl, benzofuranyl, carbazolyl, cinnolinyl, dioxolanyl, indolizinyl, naphthyridinyl, perhydroazepinyl, phenazinyl, phenothiazinyl, phenoxazinyl, phthalazinyl, pteridinyl, purinyl, quinazolinyl, quinoxalinyl, tetrazoyl, tetrahydroisoquinolyl, piperidinyl, piperazinyl, 2-oxopiperazinyl, 2-oxopiperidinyl, 2-oxopyrrolidinyl, 2-oxoazepinyl, azepinyl, 4-piperidonyl, pyrrolidinyl, pyridazinyl, oxazolinyl, oxazolidinyl, triazolyl, indanyl, isoxazolyl, isoxazolidinyl, morpholinyl, thiazolinyl, thiazolidinyl, isothiazolyl, quinuclidinyl, isothiazolidinyl, isoindolyl, indolinyl, isoindolinyl, octahydroindolyl, octahydroisoindolyl, decahydroisoquinolyl, benzimidazolyl, thiadiazolyl, benzopyranyl, tetrahydrofuryl, tetrahydropyranyl, thienyl, benzothienyl, thiamorpholinyl, thiamorpholinyl sulfoxide, thiamorpholinyl sulfone, dioxaphospholanyl, oxadiazolyl, chromanyl, and isochromanyl. The heteroaryl ring radical may be attached to the main structure at any heteroatom or carbon atom that results in the creation of a stable structure. The term “substituted heteroaryl” also includes ring systems substituted with one or more oxide (-0-) substituents, such as pyridinyl N-oxides.

The term “heteroarylalkyl” refers to a heteroaryl ring radical as defined above directly bonded to an alkyl group. The heteroarylalkyl radical may be attached to the main structure at any carbon atom from alkyl group that results in the creation of a stable structure.

The term “cyclic ring” refers to a cyclic ring containing 3 to 10 carbon atoms.

In the present application, the term “immune checkpoint” generally refers to a group of molecules on the cell surface of CD4 cells and CD8 T cells. These molecules can effectively act as “brakes” to downregulate or suppress anti-tumor immune responses Immune checkpoint molecules include but are not limited to programmed cell death 1 (PD-1), programmed cell death ligand 1 (PD-L1), programmed cell death ligand 2 (PD-L2), lymphocyte activation gene-3 (LAG-3; also known as CD223), galectin-3, B and T lymphocyte attenuator (BTLA), T-cell membrane protein 3 (TIM3), galectin-9 (GALS), B7-H1, B7-H3, B7-H4, T-cell immune receptors with Ig and ITIM domains (TIGIT/Vstm3/WUCAM/VSIG9), V-domain Ig inhibitor of T-cell activation (VISTA), Glucocorticoid-induced tumor necrosis factor receptor-related (GITR) protein, herpes virus entry medium (HVEM), OX40, CD27, CD28, CD80, CD86, CD137, CGEN-15001T, CGEN-15022, CGEN-15027, CGEN-15049, CGEN-15052, and CGEN-15092.

In the present application, the term “inhibitor of an immune checkpoint” generally refers to a molecule that inhibits, reduces or interferes with the activity of an inhibitory checkpoint molecule. Without being bound by a particular theory, inhibitory checkpoint molecules downregulate immune responses (eg, T-cell activation) by delivering negative signals to T cells upon their binding by ligands or counter-receptors. In certain embodiment, the checkpoint inhibitors used with the methods and compositions provided herein can directly inhibit the activity of inhibitory checkpoint molecules or reduce the expression of inhibitory checkpoint molecules or interfere with inhibitory checkpoint molecules and interaction of binding partners (eg, ligands).

In the present application, “inhibitors of immune checkpoints” include, but are not limited to, proteins, polypeptides, peptides, antisense oligonucleotides, antibodies, antibody fragments, or RNA molecules (e.g. that target the expression of inhibitory checkpoint molecules). inhibitory RNA molecules). Its inhibitory effect can be carried out at the DNA, RNA or protein level. For example, inhibitory nucleic acids (eg, dsRNA, siRNA, or shRNA) can be used to inhibit the expression of inhibitory molecules. In other embodiment, the inhibitor of the inhibitory signal is a polypeptide that binds to an immune checkpoint, e.g., a soluble ligand (e.g., PD-1-Ig), an antibody or an antigen-binding fragment thereof; e.g., with PD-1, PD—An antibody or fragment thereof that binds to L1, PD-L2, CTLA4, TIM3, LAGS, VISTA, BTLA, TIGIT, LAIR1, CD160, 2B4 and/or TGFRβ or a combination thereof.

In the present application, the term“TGFβRII” or“TGFβ Receptor II” is meant a polypeptide having the wild-type human TGFβ Receptor Type 2 Isoform A sequence (e.g., the amino acid sequence of NCBI Reference Sequence (RefSeq) Accession No. NP_001020018 (SEQ ID NO.8)), or a polypeptide having the wild-type human TGFβ Receptor Type 2 Isoform B sequence (e.g., the amino acid sequence of NCBI RefSeq Accession No. NP_003233 (SEQ ID NO.9)) or having a sequence substantially identical the amino acid sequence of SEQ ID NO.8 or of SEQ ID NO. 9. The TGFβRII may retain at least 0.1%, 0.5%, 1%, 5%, 10%, 25%, 35%, 50%, 75%, 90%, 95%, or 99% of the TGFβ-binding activity of the wild-type sequence.

In the present application, the term“fragment of TGFβRII capable of binding TGFβ” is meant any portion of NCBI RefSeq Accession No. NP_001020018 (SEQ ID NO.8) or of NCBI RefSeq Accession No. NP_003233 (SEQ ID NO.9), or a sequence substantially identical to SEQ ID NO.8 or SEQ ID NO.9 that is at least 20 (e.g., at least 30, 40, 50, 60, 70, 80, 90, 100, 110, 120, 130, 140, 150, 160, 175, or 200) amino acids in length that retains at least some of the TGFβ-binding activity (e.g., at least 0.1%, 0.5%, 1%, 5%, 10%, 25%, 35%, 50%, 75%, 90%, 95%, or 99%) of the wild-type receptor or of the corresponding wild-type fragment. Typically such fragment is a soluble fragment. An exemplary such fragment is a TGFβRII extra-cellular domain having the sequence of SEQ ID NO: 6

In the present application, the term“substantially identical” is meant a polypeptide exhibiting at least 50%, desirably 60%, 70%, 75%, or 80%, more desirably 85%, 90%, or 95%, and most desirably 99% amino acid sequence identity to a reference amino acid sequence. The length of comparison sequences will generally be at least 10 amino acids, desirably at least 15 contiguous amino acids, more desirably at least 20, 25, 50, 75, 90, 100, 150, 200, 250, 300, or 350 contiguous amino acids, and most desirably the full-length amino acid sequence. For example, suitable algorithms for determining percent sequence identity and percent sequence similarity may be the BLAST and BLAST 2.0 algorithms, respectively, see Altschul et al. (1977) Nucleic Acids Res. 25:3389 and Altschul et al. (1990) J. Mol. Biol. 215:403.

In the present application, the term “TGFβ inhibitor” generally refers to substances that inhibit the production and signal transduction of TGFβ signal molecules, and the inhibitors can be small molecular compounds or macromolecular compounds (such as antibodies). Exemplary small molecule TGFβ inhibitors include SB525334, SD-208, SB431542, LY2109761, LY2157299 (Galunisertib), GW788388, RepSox, SIS3, LDN-193189, EW-7197, and LY364947, among others.

| Compound | Chemical name |
|---|---|
| SB525334 | 6-(2-(Tert-Butyl)-5-(6-methylpyridin-2-yl)-1H-imidazol-4-yl)quinoxaline |
| SD-208 | 2-(5-Chloro-2-fluorophenyl)-N-(pyridin-4-yl)pteridin-4-amine |
| SB431542 | 4-(4-(Benzo[d][1,3]dioxol-5-yl)-5-(pyridin-2-yl)-1H-imidazol-2-yl)benzamide |
| LY2109761 | 4-(2-((4-(2-(Pyridin-2-yl)-5,6-dihydro-4H-pyrrolo[1,2-b]pyrazol-3-yl)quinolin-7- |
| | yl)oxy)ethyl)morpholine |
| LY2157299 | 4-[2-(6-methylpyridin-2-y1)-5,6-dihydro-4H-pyrrolo[1,2-b]pyrazol-3- |
| | yl]quinoline-6-carboxamide |
| GW788388 | N-(oxan-4-y1)-4-[4-(5-pyridin-2-yl-1H-pyrazol-4-y1)pyridin-2-yl]benzamide |
| RepSox | 2-[5-(6-methylpyridin-2-yl)-1H-pyrazol-4-y1]-1,5-naphthyridine |
| SIS3 | (E)-1-(6,7-dimethoxy-3,4-dihydro-1H-isoquinolin-2-y1)-3-(1-methyl-2- |
| | phenylpyrrolo[2,3-b]pyridin-3-yl)prop-2-en-1-one; hydrochloride |
| LDN-193189 | 4-[6-(4-piperazin-1-ylphenyl)pyrazolo[1,5-a]pyrimidin-3-y]]quinoline |
| EW-7197 | 2-fluoro-N-[[5-(6-methylpyridin-2-yl)-4-([1,2,4]triazolo[1,5-a]pyridin-6-yl)-1H- |
| | imidazol-2-yl]methyl]aniline |
| LY364947 | 4-(5-pyridin-2-yl-1H-pyrazol-4-yl)quinoline |

In the present application, the term “antibody” generally refers to immunoglobulins that are reactive to a specified protein or peptide or fragment thereof. Antibodies may be antibodies from any class, including but not limited to IgG, IgA, IgM, IgD and IgE, and antibodies from any subclass (e.g., IgG1, IgG2, IgG3, and IgG4). The antibody may have a heavy chain constant region selected from the group consisting of, for example, IgG1, IgG2, IgG3, or IgG4. The antibody may also have a light chain selected from the group consisting of, for example, kappa (kappa) or lambda (lambda). The antibody of the application can be derived from any species.

In the present application, the term “antigen-binding fragment” generally refers to a portion of an antibody molecule, which comprises an amino acid responsible for the specific binding between the antibody and the antigen. The portion of the antigen that is specifically recognized and bound by the antibody is referred to as the “epitope” as described above. The antigen binding domain may typically comprise an antibody light chain variable region (VL) and an antibody heavy chain variable region (VH); However, it is not necessary to contain both. The Fd fragment, for example, has two VH regions and generally retains some antigen binding function of the entire antigen-binding domain. Examples of antigen-binding fragments of an antibody include (1) a Fab fragment, a monovalent fragment having VL, VH, constant light chain (CL) and CH1 domain; (2) F (ab′) 2 fragment, a divalent fragment of two Fab fragments connected by disulfide bridge of the hinge region; (3) Fd fragments having two VH and CH1 domains; (4) Fv fragment with VL and VH domain of antibody single arm; (5) dAb fragment (Ward et al., “ Binding Actida of a Repertoire of Single Immunoglobulin. variable Domains Secreted From Escherichia coli, Nature 341: 544-546 (1989), which is incorporated herein by reference), which has a VH domain; (6) a separate complementarity determining region (CDR); (7) single chain Fv (scFv), such as from scFV-library. Although the two domains of the Fv fragment, VL and VH, are encoded by an independent gene, they can be joined by a recombinant method using a synthetic linker; the synthetic linker is prepared into a single protein chain (called single chain Fv (scFv)) wherein VL and VH regions are paired to form monovalent molecules (referred to as single chain Fv (scFv)) (see e.g. Huston et al., Protein Engineering of Antibody Binding Sites: Recovery of SpecificActivity in an Anti-Digoxin Single-Chain Fv Analogue Produced in Escherichia coli, ” Proc. Natl. Acad. Sci. USA 85: 5879-5883 (1988)); and (8) VHH, VHH refers to a camel (camel, single-peak camel, llama, alpaca, etc.) heavy chain antibody of variable antigen-binding domain (see Nguyen V. K. et al., 2000, The EMBO Journal, 19, 921-930; Muyldermans S., 2001, J Biotechnol, 74, 277-302, and the Vanlandschoot P. et al., 2011, Antiviral Research 92, 389-407). VHH also can be referred to as Nanobody (Nanobody) (Nb) and/or single domain antibody. These antibody fragments are obtained using conventional techniques known to those of ordinary skill in the art, and the function of the fragments is evaluated in the same manner as the intact antibody.

In the present application, the term “variable region” or “variable domain” generally refers to a domain of an antibody heavy or light chain involved in the binding of an antibody to an antigen. In the present application, the term “variable” generally refers to some part of the sequence of the variable domain of the antibody is strong, forming various specific antibody binding and specificity of the specific antigen. The variability is non-uniformly distributed throughout the variable region of the antibody. which is concentrated in three sections of light chain variable region and heavy chain variable region, called complementarity determining region (CDR) or hypervariable region (HVR), respectively is LCDR1, LCDR2, LCDR3, HCDR1, HCDR2 and HCDR3. A more highly conserved portion in the variable domain is referred to as a framework region (FR). The variable domains of the natural heavy chain and the light chain each comprise four FR regions (H-FR1, H-FR2, H-FR3, H-FR4, L-FR1, L-FR2, L-FR3 and L-FR4), most of which are in the beta-folded configuration, and are connected by three CDR structural ring regions. the CDRs in each strand are closely adjacent together through the FR region and form the antigen binding site of the antibody together with the CDRs from the other strand.

In the present application, the variable region of the antibody can be encoded by a variety of methods or CDRs of a divided antibody, such as Kabat numbering scheme and definition rule (see, Kabat et al, Immunology protein sequence, Fifth Edition, United States National Institution of Health, Besseda, based on sequence variability. Maryland (1991)), Chothia numbering scheme based on structural ring region position and defining rule (see, Al-Lazikani et al., JMol Biol 273: 927-48, 1997); IMGT numbering scheme and definition rule of efranc et al based V gene based on the amino acid sequence, and Honneger's numbering scheme (AHo's), Martin numbering scheme, Gelfand numbering scheme and so on, can refer to Mathieu Dondelinger et al., Understanding the Significance and Imperial of Antibody Numbering and Antigen-Binding Surface/Residue Definition, Front. Immunol., 16 Oct. 2018.

In the present application, “peptide linker” generally refers to the amino acid sequence through which the amino acid sequences of different structural domains in the PD-L 1/TGFβ dual inhibitor of the present application are connected to each other. An essential technical feature of this peptide linker is that the peptide linker does not contain any polymerization activity. Preferred amino acid residues for peptide linkers include Gly, Ser and Thr, characterized by a length of 5 to 25 amino acid residues. Suitable peptide linkers include those described in U.S. Pat. Nos. 4,751,180 and 4,935,233 or WO88/09344. A preferred embodiment of the peptide linker is characterized by the amino acid sequence Gly-Gly-Gly-Gly-Ser, ie Gly4Ser, or a polymer thereof, ie (Gly4Ser)x, where x is an integer 1 or greater. The characteristics of such peptide linkers, including not promoting secondary structure, are known in the art and described, for example, in Dall'Acqua et al. (Biochem. (1998) 37, 9266-9273), Cheadle et al. (Mol Immunol (1992) 29, 21-30) and Raag and Whitlow (FASEB (1995) 9(1), 73-80). Peptide linkers that also do not promote any secondary structure are preferred. Linkage of the domains to each other is provided, eg by genetic engineering, as described in the Examples. Methods for preparing fused and operably linked bispecific single chain constructs and expressing them in mammalian cells or bacteria are well known in the art (e.g. WO99/54440 or Sambrook et al., Molecular Cloning: A Laboratory Manual, Cold Spring Harbor Laboratory Press, Cold Spring Harbor, New York, 2001).

In the present application, the terms “antagonist” and “inhibitor” are used interchangeably, and generally refer to a compound or compound capable of reducing or inhibiting the biological function of a target protein or polypeptide, such as reducing or inhibiting the activity or expression of a target protein or polypeptide. potion. Inhibitors need not completely abolish the biological function of the target protein or polypeptide, and In certain embodiment, reduce activity by at least 50%, 60%, 70%, 80%, 90%, 95%, or 99%. While some antagonists of the present application specifically interact with (e.g., bind to) a target, inhibit the biological activity of the target protein or polypeptide by interacting with other members of a signal transduction pathway that includes the target protein or polypeptide Compounds of are also specifically included in this definition. Non-limiting examples of biological activities inhibited by antagonists include those associated with the development, growth or spread of tumors, or undesired immune responses manifested in autoimmune diseases.

In the present application, the term “effective amount” or “therapeutically effective amount” generally refers to the amount of the compound or pharmaceutical composition described in the application that is sufficient to achieve the intended application described below, including but not limited to disease treatment. The therapeutically effective amount can vary according to: the intended application (in vivo or in vitro); or the subject and the condition to be treated, for example, the weight and age of the subject, the severity of the condition; the mode of administration, etc., which can be determined by ordinary skill in the art Personnel are easily identified. The terms also apply to doses that will induce a specific response in target cells, such as platelet adhesion and/or cell migration. The specific dosage will vary according to, for example, the particular compound chosen, the dosing regimen followed, whether it is administered in combination with other agents, the time of administration, the tissue to which it is administered, and the physical delivery system by which it is delivered.

In the present application, the term “in vivo” generally refers to events that occur within the body of a subject.

In the present application, the term “in vitro” generally refers to events that occur outside the body of a subject. For example, an in vitro assay includes any assay performed outside of a subject. In vitro assays include cell-based assays in which live or dead cells are employed. In vitro assays also include cell-free assays, in which intact cells are not used

In the present application, the term “combination therapy” or “combination” generally refers to the use of more than one compound or agent to treat a particular disorder or condition. For example, a PI3K inhibitor can be administered in combination with at least one additional therapeutic agent. “Combination” is not intended to imply that the other therapy and the PI3K inhibitor must be administered and/or formulated for delivery together, but such methods of delivery are within the scope of this application. The PI3K inhibitor can be administered simultaneously with, prior to (e.g., 5 minutes, 15 minutes, 30 minutes, 45 minutes, 1 hour, 2 hours, 4 hours, 6 hours, 12 hours, 24 hours) the one or more additional agents hours, 48 hours, 72 hours, 96 hours, 1 week, 2 weeks, 3 weeks, 4 weeks, 5 weeks, 6 weeks, 8 weeks, 12 weeks or 16 weeks before) or after (for example, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks or 16 weeks later) administration. Generally, each therapeutic agent will be administered at a dosage and/or on a schedule established for that particular agent. Other therapeutic agents can be administered with the PI3K inhibitors provided herein in a single composition or separately in different compositions. This application also considers higher combinations, such as triple therapy of PI3K inhibitor+PD-L1 inhibitor+TGFβ inhibitor.

Combination therapies term “administering” generally refers to introducing the drug combination into the body of a subject by any route of introduction or delivery. Any method known to those skilled in the art for contacting cells, organs or tissues with the drug combination may be employed. Including but not limited to intraarterial, intranasal, intraperitoneal, intravenous, intramuscular, subcutaneous transdermal or oral. The daily dose may be divided into one, two or more doses in suitable form to be administered at one, two or more times during a certain period of time.

In certain embodiment, the term “administering” includes administering two or more agents to a subject such that the agents and/or metabolites thereof are present in the subject simultaneously or substantially simultaneously. In certain embodiment, co-administration of a PI3K inhibitor with an additional anticancer agent (both components hereinafter referred to as “two active agents”) refers to any administration of the two active agents, either separately or together, Wherein the two active agents are administered as part of a suitable dosage regimen aimed at obtaining the benefits of combination therapy. Thus, the two active agents may be administered as part of the same pharmaceutical composition or in separate pharmaceutical compositions. The additional agent may be administered before, simultaneously with, or after the administration of the PI3K inhibitor, or in some combination thereof. When a PI3K inhibitor is administered to a patient at repeated intervals, for example, during a standard course of treatment, the additional agent may be administered before, simultaneously with, or after each administration of the PI3K inhibitor, or in some combination thereof, or in a relative PI3K inhibiting The doses are administered at different intervals of treatment, or administered as a single dose before, at any time during the course of treatment with the PI3K inhibitor, or after the course of treatment with the PI3K inhibitor. In certain embodiment, the first agent can be administered before (e.g., 5 minutes, 15 minutes, 30 minutes, 45 minutes, 1 hour, 2 hours, 4 hours, 6 hours, 12 hours, 24 hours) the administration of the second therapeutic agent, 48 hours, 72 hours, 96 hours, 1 week, 2 weeks, 3 weeks, 4 weeks, 5 weeks, 6 weeks, 8 weeks, or 12 weeks ago), substantially at the same time or after (for example, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks or 12 weeks later) administration.

In the present application, “monotherapy” refers to the use of an agent (for example, as a single compound or agent) alone (also referred to herein as “single (alone)”), for example in the absence of a second active ingredients to treat the same indication, for example cancer. In this context, the term “monotherapy” includes the use of a PI3K single agent or a second agent alone in the treatment of cancer.

In the present application, the term “synergy” or “synergistic” includes the additive effect of the combination of two or more agents that is higher than the individual effects of the two or more agents. In certain embodiment, synergy or synergistic effect refers to the beneficial effect of two or more agents used in combination, e.g., in a pharmaceutical composition or in a method of treatment. In certain embodiment, one or more beneficial effects are achieved by using a PI3K inhibitor in combination with a second therapeutic agent (e.g., one or more second therapeutic agents) described herein.

In certain embodiment, synergy means that a lower dose of one or both agents is required to achieve an effect. For example, a combination may provide a selected effect, such as a therapeutic effect, when at least one agent is administered at a lower dose than that required to achieve the same therapeutic effect when the agent is administered as a monotherapy. In certain embodiment, the combination of a PI3K inhibitor (e.g., a PI3K inhibitor) and a second agent (as described herein) allows the PI3K inhibitor to achieve the same therapeutic effect than when the PI3K inhibitor is administered as monotherapy. Administer at lower doses as required.

In certain embodiment, the synergistic effect reduces, prevents, delays or reduces the occurrence or likelihood of one or more side effects, toxicity, resistance otherwise associated with the administration of at least one of the agents.

In certain embodiment, the synergistic effect reduces resistance to at least one of the agents (e.g., reduces the measure of resistance or reduces the likelihood of developing resistance) or delays the development of resistance to at least one of the agents produce.

In certain embodiment, the synergistic effect is a reduction in minimal residual disease (MRD). In certain embodiment, the combination of a PI3K inhibitor (e.g., a PI3K inhibitor described herein) and a second agent (e.g., a second agent described herein) is effective to reduce MRD in a subject, e.g., low levels previously measured in a subject (e.g., levels measured prior to administration of the combination). In certain embodiment, the combination of a PI3K inhibitor and a second agent is effective to reduce MRD in a subject below levels observed during or after treatment with a monotherapy comprising, for example, a PI3K inhibitor or a second agent. Monotherapy with any of the agents. In certain embodiment, the MRD is reduced below levels observed during treatment with a monotherapy comprising a PI3K inhibitor. In certain embodiment, the MRD is reduced below the level observed during treatment with the monotherapy comprising the second agent. In certain embodiment, the combination is effective to reduce the level of MRD to a preselected cutoff value (e.g., 1 malignant cell in 100 normal cells, 1 malignant cell in 1000 normal cells, or 10,000 normal cells 1 malignant tumor cell per cell, or 1 malignant tumor cell per 100,000 normal cells). In certain embodiment, the preselected cutoff value is 1 malignant cell in 1000 normal cells. In certain embodiment, the preselected cutoff value is 1 malignant cell in 100,000 normal cells.

In certain embodiment, synergy refers to a PI3K inhibitor (e.g., a PI3K inhibitor or a pharmaceutically acceptable form thereof) and a second therapeutic agent (e.g., one or more additional therapeutic agents or a pharmaceutically acceptable form thereof). The combination produces a higher therapeutic effect than the additive effect of the PI3K inhibitor and the second agent.

In the present application, the term “pharmaceutically acceptable” generally refers to one or more non-toxic substances that do not interfere with the effectiveness of the biological activity of the active ingredient. Such formulations will generally contain salts, buffers, preservatives, compatible carriers and optionally other therapeutic agents.

In the present application, “pharmaceutically acceptable form” of a compound includes, but is not limited to, pharmaceutically acceptable salts, hydrates, solvates, isomers, prodrugs and isotope-labeled derivatives of the disclosed compounds. In certain embodiment, “pharmaceutically acceptable form” includes, but is not limited to, pharmaceutically acceptable salts, isomers, prodrugs, and isotopically labeled derivatives of the disclosed compounds.

In certain embodiment, the pharmaceutically acceptable form is a pharmaceutically acceptable salt. As used herein, the term “pharmaceutically acceptable salt” refers to those salts which are, within the scope of sound medical judgment, suitable for use in contact with the tissues of subjects without undue toxicity, irritation, allergic response and the like, and are commensurate with a reasonable benefit/risk ratio. Pharmaceutically acceptable salts are well known in the art. For example, Berge et al. describes pharmaceutically acceptable salts in detail v& J. Pharmaceutical Sciences (1977) 66: 1-19. Pharmaceutically acceptable salts of the compounds provided herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, besylate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. In certain embodiment, organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

In certain embodiment, the pharmaceutically acceptable form is a pharmaceutically acceptable salt. As used herein, the term “pharmaceutically acceptable salt” refers to those salts which are, within the scope of sound medical judgment, suitable for use in contact with the tissues of subjects without undue toxicity, irritation, allergic response and the like, and are commensurate with a reasonable benefit/risk ratio. Pharmaceutically acceptable salts are well known in the art. For example, Berge et al. describes pharmaceutically acceptable salts in detail v & J. Pharmaceutical Sciences (1977) 66: 1-19. Pharmaceutically acceptable salts of the compounds provided herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, besylate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. In certain embodiment, organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

In certain embodiment, the term “solvate” refers to compounds that further include a stoichiometric or non-stoichiometric amount of solvent bound by non-covalent intermolecular forces. The solvate can be of a disclosed compound or a pharmaceutically acceptable salt thereof. Where the solvent is water, the solvate is a “hydrate”. Pharmaceutically acceptable solvates and hydrates are complexes that, for example, can include 1 to about 100, or 1 to about 10, or 1 to about 2, about 3 or about 4, solvent or water molecules. It will be understood that the term “compound” as used herein encompasses the compound and solvates of the compound, as well as mixtures thereof.

In certain embodiment, the term “prodrug” refers to compounds that are transformed in vivo to yield a disclosed compound or a pharmaceutically acceptable form of the compound. A prodrug can be inactive when administered to a subject, but is converted in vivo to an active compound, for example, by hydrolysis (e.g., hydrolysis in blood). In certain cases, a prodrug has improved physical and/or delivery properties over the parent compound. Prodrugs are typically designed to enhance pharmaceutically and/or pharmacokinetic ally based properties associated with the parent compound. The prodrug compound often offers advantages of solubility, tissue compatibility or delayed release in a mammalian organism (see, e.g., Bundgard, H., Design of Prodrugs (1985), pp. 7-9, 21-24 (Elsevier, Amsterdam). A discussion of prodrugs is provided in Higuchi, T., et al., “Pro-drugs as Novel Delivery Systems,” A. C.S. Symposium Series, Vol. 14, and in Bioreversible Carriers in Drug Design, ed. Edward B. Roche, American Pharmaceutical Association and Pergamon Press, 1987, both of which are incorporated in full by reference herein. Exemplary advantages of a prodrug can include, but are not limited to, its physical properties, such as enhanced water solubility for parenteral administration at physiological pH compared to the parent compound, or it enhances absorption from the digestive tract, or it can enhance drug stability for long-term storage.

The term “prodrug” is also meant to include any covalently bonded carriers, which release the active compound in vivo when such prodrug is administered to a subject. Prodrugs of an active compound, as described herein, can be prepared by modifying functional groups present in the active compound in such a way that the modifications are cleaved, either in routine manipulation or in vivo, to the parent active compound. Prodrugs include compounds wherein a hydroxy, amino or mercapto group is bonded to any group that, when the prodrug of the active compound is administered to a subject, cleaves to form a free hydroxy, free amino or free mercapto group, respectively. Examples of prodrugs include, but are not limited to, acetate, formate and benzoate derivatives of an alcohol or acetamide, formamide and benzamide derivatives of an amine functional group in the active compound and the like. Other examples of prodrugs include compounds that comprise —NO, —N02, —ONO, or —ON02 moieties. Prodrugs can typically be prepared using well-known methods, such as those described in Burger's Medicinal Chemistry and Drug Discovery, 172-178, 949-982 (Manfred E. Wolff ed., 5th ed., 1995), and Design of Prodrugs (H. Bundgaard ed., Elselvier, New York, 1985).

For example, if a disclosed compound or a pharmaceutically acceptable form of the compound contains a carboxylic acid functional group, a prodrug can comprise a pharmaceutically acceptable ester formed by the replacement of the hydrogen atom of the acid group with a group such as (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N-(C₁-C₂)alkylamino(C₂-C₃)alkyl (such as β-dimethylaminoethyl), carbamoyl-(C₁-C₂)alkyl, N,N-di(C₁-C₂)alkylcarbamoyl-(C₁-C₂)alkyl and piperidino-, pyrrolidino- or mo holino(C₁-C₃)alkyl.

Similarly, if a disclosed compound or a pharmaceutically acceptable form of the compound contains an alcohol functional group, a prodrug can be formed by the replacement of the hydrogen atom of the alcohol group with a group such as (C₁-C₆)alkanoyloxymethyl, 1-((C₁-C₆)alkanoyloxylethyl, 1-methyl-1-((C₁-C₆)alkanoyloxy)ethyl (C₁-C₆)alkoxycarbonyloxymethyl, N-(C₁-C₆)alkoxycarbonylaminomethyl, succinoyl, (C₁-C₆)alkanoyl, a-amino(C₁-C₄)alkanoyl, arylacyl and a-aminoacyl, or α-aminoacyl-a-aminoacyl, where each a-aminoacyl group is independently selected from the naturally occurring L-amino acids, P(O)(OH)₂, —P(O)(O(C₁-C₆)alkyl)₂ or glycosyl (the radical resulting from the removal of a hydroxyl group of the hemiacetal form of a carbohydrate).

If a disclosed compound or a pharmaceutically acceptable form of the compound incorporates an amine functional group, a prodrug can be formed by the replacement of a hydrogen atom in the amine group with a group such as R-carbonyl, RO-carbonyl, NRR′-carbonyl where R and R′ are each independently (C₁-C₁₀)alkyl, (C₃-C₇)cycloalkyl, benzyl, a natural α-aminoacyl or natural a-aminoacyl-natural α-aminoacyl, —C(OH)C(O)OY¹ wherein Y¹ is H, (C₁-C₆)alkyl or benzyl, —C(OY²)Y³ wherein Y² is (C₁-C₄) alkyl and Y³ is (C₁-C₆)alkyl, carboxy(C₁-C₆)alkyl , amino(C₁-C₄)alkyl , or mono-N- or di-N,N—(C₁-C₆)alkylaminoalkyl, C(Y⁴)Y⁵ wherein Y⁴ is H or methyl and Y⁵ is mono-N-or di-N,N—(C₁-C₆)alkyl amino, morpholino, piperidin-1-yl or pyrrolidin-1-yl.

In certain embodiment, “isomers” are different compounds that have the same molecular formula. “Stereoisomers” are isomers that differ only in the way the atoms are arranged in space. As used herein, the term “isomer” includes any and all geometric isomers and stereoisomers. For example, “isomers” include geometric double bond cis- and trans-isomers, also termed E- and Z-isomers; R- and S-enantiomers; diastereomers, (d)-isomers and (l)-isomers, racemic mixtures thereof; and other mixtures thereof, as falling within the scope of this disclosure.

“Enantiomers” are a pair of stereoisomers that are non-superimposable mirror images of each other. A mixture of a pair of enantiomers in any proportion can be known as a “racemic” mixture. The term “(±)” is used to designate a racemic mixture where appropriate. “Diastereoisomers” are stereoisomers that have at least two asymmetric atoms, but which are not mirror-images of each other. The absolute stereochemistry is specified according to the Cahn-Ingold-Prelog R-S system. When a compound is an enantiomer, the stereochemistry at each chiral carbon can be specified by either R or S. Resolved compounds whose absolute configuration is unknown can be designated (+) or (−) depending on the direction (dextro- or levorotatory) which they rotate plane polarized light at the wavelength of the sodium D line. Certain of the compounds described herein contain one or more asymmetric centers and can thus give rise to enantiomers, diastereomers, and other stereoisomeric forms that can be defined, in terms of absolute stereochemistry at each asymmetric atom, as (R)- or (S)-. The present chemical entities, pharmaceutical compositions and methods are meant to include all such possible isomers, including racemic mixtures, optically substantially pure forms and intermediate mixtures. Optically active (R)- and (S)-isomers can be prepared, for example, using chiral synthons or chiral reagents, or resolved using conventional techniques. When the compounds described herein contain olefinic double bonds or other centers of geometric asymmetry, and unless otherwise indicated, it is intended that the compounds include both E and Z geometric isomers.

“Enantiomeric purity” as used herein refers to the relative amounts, expressed as a percentage, of the presence of a specific enantiomer relative to the other enantiomer. For example, if a compound, which can potentially have an (R)- or an (S)-isomeric configuration, is present as a racemic mixture, the enantiomeric purity is about 50% with respect to either the (R)- or (S)- isomer. If that compound has one isomeric form predominant over the other, for example, 80% (S)- and 20% (R)-, the enantiomeric purity of the compound with respect to the (S)-isomeric form is 80%. The enantiomeric purity of a compound can be determined in a number of ways known in the art, including but not limited to chromatography using a chiral support, polarimetric measurement of the rotation of polarized light, nuclear magnetic resonance spectroscopy using chiral shift reagents which include but are not limited to lanthanide containing chiral complexes or the Pirkle alcohol, or derivatization of a compounds using a chiral compound such as Mosher's acid followed by chromatography or nuclear magnetic resonance spectroscopy.

In certain embodiment, the term “tautomer” is a type of isomer that includes two or more interconvertable compounds resulting from at least one formal migration of a hydrogen atom and at least one change in valency {e.g., a single bond to a double bond a triple bond to a single bond, or vice versa). “Tautomerization” includes prototropic or proton-shift tautomerization, which is considered a subset of acid-base chemistry. “Prototropic tautomerization” “proton-shift tautomerization” involves the migration of a proton accompanied by changes in bond order. The exact ratio of the tautomers depends on several factors, including temperature, solvent, and pH. Where tautomerization is possible {e.g., in solution), a chemical equilibrium of tautomers can be reached. Tautomerizations {i.e., the reaction providing a tautomeric pair) can be catalyzed by acid or base, or can occur without the action or presence of an external agent. Exemplary tautomerizations include, but are not limited to, keto-to-enol; amide-to-imide; lactam-to-lactim; enamine-to-imine; and enamine-to-(a different) enamine tautomerizations. A specific example of keto-enol tautomerization is the interconversion of pentane-2,4-dione and 4-hyydroxypent-3-en-2-one tautomers. Another example of tautomerization is phenol-keto tautomerization. A specific example of phenol-keto tautomerization is the interconversion of pyridin-4-ol and pyridin-4(1H)-one lautomers.

Unless otherwise stated, structures depicted herein are also meant to include compounds which differ only in the presence of one or more isotopically enriched atoms. For example, compounds having the present strictures except for the replacement of a hydrogen by a deuterium or tritium, or the replacement of a carbon by ¹³C- or ¹⁴C-enriched carbon are within the scope of this disclosure.

The disclosure also embraces isotopically labeled compounds which are identical to those recited herein, except that one or more atoms are replaced by an atom having an atomic mass or mass number different from the atomic mass or mass number usually found in nature, Examples of isotopes that can be incorporated into disclosed compounds include isotopes of hydrogen, carbon, nitrogen, oxygen, phosphorus, fluorine and chlorine, such as ²H, ³H, ¹³C, ¹⁴C, ¹⁵, ¹⁸O, ¹⁷O, ³¹P, ³²P, ³⁵S, ¹⁸F, and ³⁶Cl, respectively. Certain isotopically labeled disclosed compounds (e.g., those labeled with ³H and ¹⁴C) are useful in compound and/or substrate tissue distribution assays. Tritiated (i.e., ³H) and carbon-14 (i.e., ¹⁴C) isotopes can allow for ease of preparation and detectability. Further, substitution with heavier isotopes such as deuterium e., ²H) can afford certain therapeutic advantages resulting from greater metabolic stability (e.g., increased in vivo half-life or reduced dosage requirements). Isotopically labeled disclosed compounds can generally be prepared by substituting an isotopically labeled reagent for a non-isopically labeled reagent. In certain embodiment, provided herein are compounds that can also contain unnatural proportions of atomic isotopes at one or more of atoms that constitute such compounds. All isotopic variations of the compounds as provided herein, whether radioactive or not, are encompassed within the scope of the present disclosure.

“Pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions as provided heroin is contemplated. Supplementary active ingredients can also be incorporated into the pharmaceutical compositions.

In the present application, the term “tumor” generally refers to any neoplastic cell growth and proliferation, malignant or benign, as well as any pre-cancerous and cancerous cells and tissues. In the present application, the term “neoplastic” refers to any form of abnormally regulated or unregulated cell growth, malignant or benign, resulting in abnormal tissue growth. Thus, “neoplastic cells” include malignant and benign cells with abnormally regulated or unregulated cell growth. The terms “cancer”, “cancerous”, “cell proliferative disorder”, “proliferative disorder” and “tumor” are not mutually exclusive when referred to In the present application. In certain embodiment, a tumor may refer to a mass of flesh containing a plurality of cancer cells, e.g, cells exhibiting characteristics of any of the cancers described In the present application. In the present application, a tumor may be a solid tumor or a non-solid tumor (eg, hematological tumor, lymphoma). The term “solid tumor” generally refers to an abnormal growth or mass of tissue that usually does not contain cysts or fluid areas. Solid tumors can be benign (noncancerous) or malignant (cancerous). in the present application, the tumor may be a tumor with abnormal expression of PD-1 or PD-L1 in cells and tissues. In the present application, the tumor may be a tumor with abnormal expression or abnormal activity of TGFβ or TGFβR in cells and tissues. In the present application, the tumor may be a tumor with abnormal expression or activity of PI3K in cells and tissues.

In the present application, the term refers to a point where cancer has a reduced response to the treatment, for example, until the cancer does not respond to treatment. Cancer can be resistant at the beginning of treatment or it becomes resistant during the treatment. The cancer subject may have one or more mutations caused to become resistant to treatment, or subject can form such mutation during the treatment. The term “refractory” can refer to cancers for which treatment (e.g., chemotherapeutic drugs, biologics, and/or radiation therapy) has proven ineffective. Refractory cancers can shrink, but not to the extent that treatment is definitively effective. Typically, however, the tumor remains the same size as it was before treatment (stable disease), or grows (progressive disease).

In the present application, the terms “treatment” and “treating” generally refer to a method of obtaining a beneficial or desired result, including, but not limited to, a therapeutic benefit. A therapeutic benefit includes, but is not limited to, eradicating, inhibiting, reducing or ameliorating the underlying disorder being treated. Additionally, therapeutic benefit is achieved by eradicating, reducing or ameliorating one or more physiological symptoms associated with the underlying disorder such that improvement is observed in a patient, but the patient may still suffer from the underlying disorder.

In the present application, the terms “prevention” and “preventing” generally refer to methods of obtaining beneficial or desired results, including but not limited to prophylactic benefits. For prophylactic benefit, pharmaceutical compositions may be administered to patients at risk of developing a particular disease or to patients reporting one or more physical symptoms of a disease, even if the disease has not yet been diagnosed.

The term “subject” or “patient” is contemplated to humans (i.e., a male or female of any age group, e.g., a pediatric subject (e.g., infant, child, adolescent) or adult subject (e.g., young adult, middle-aged adult or senior adult)) and/or other primates (e.g., cynomolgus monkeys, rhesus monkeys); mammals, including commercially relevant mammals such as cattle, pigs, horses, sheep, goats, cats, and/or dogs; and/or birds, including commercially relevant birds such as chickens, ducks, geese, quail, and/or turkeys.

In the present application, the terms “about” or “approximately” generally refer to an acceptable error for a particular value as determined by one of ordinary skill in the art, depending in part on the manner in which the value was measured or determined. In certain embodiment, the term “about” or “approximately” generally refers to 1, 2, 3 or 4 standard deviations. In certain embodiment, the term “about” or “approximately” generally refers to 50%, 20%, 15%, 10%, 9%, 8%, 7%, 6%, 5% of a given value or range, 4%, 3%, 2%, 1%, 0.5% or 0.05%.

In the present application, the term “comprising” or “comprising” generally refers to an open form, and it should be understood that other substances not mentioned may also be included. For example, a drug combination comprising a phosphoinositide 3-kinase (PI3K) inhibitor and a second therapeutic agent, wherein the second therapeutic agent is an inhibitor of an immune checkpoint, a TGFβ inhibitor, a bifunctional immune checkpoint/TGFβ inhibitors or combinations thereof. Wherein, “comprising” should be understood as containing other substances besides the phosphoinositide 3-kinase (PI3K) inhibitor and the second therapeutic agent.

### Details of Invention

### Pharmaceutical Combination

For one aspect, the present application provides a pharmaceutical combination comprising a phosphoinositide 3-kinase (PI3K) inhibitor and a second therapeutic agent, wherein the second therapeutic agent is an immune checkpoint inhibitor, a TGFβ inhibitor, a bifunctional immune checkpoint/TGFβ inhibitors or combinations thereof.

In certain embodiment, wherein the immune checkpoint inhibitor comprises an agent capable of blocking the interaction between programmed death 1 (PD-1) and programmed death ligand 1 (PD-L1).

In certain embodiment, wherein the immune checkpoint inhibitor comprises a programmed death ligand 1 (PD-L1) and/or programmed death 1 (PD-1) inhibitor. For example, human PD-1 inhibitor, for example human PD-L1 inhibitor. As another example, the immune checkpoint inhibitor may comprise an anti-PD-L1 antibody or an antigen-binding fragment thereof, or an anti-PD-1 antibody or an antigen-binding fragment thereof. In certain embodiment, the inhibitor of PD-1 or PD-L1 can be an antibody molecule against PD-1 or PD-Ll. PD-1 or PD-L1 inhibitors can be administered alone or in combination with other immune checkpoint modulators, for example, with inhibitors of LAG-3, TIM-3, or CTLA-4.

In certain embodiment, wherein the combination comprises:
1) the combination of PD-L1 inhibitor and PI3K inhibitor;
2) the combination of TGFβ inhibitor and PI3K inhibitor;
3) the combination of PD-1 inhibitor, TGFβ inhibitor and PI3K inhibitor;
4) the combination of PD-L1 inhibitor, TGFβ inhibitor and PI3K inhibitor;
5) the combination of PD-L1/TGFβ dual inhibitor and PI3K inhibitor; or
6) the combination of PD-1/TGFβ dual inhibitor and PI3K inhibitor.

In certain embodiment, wherein the inhibitors of immune checkpoints include nucleic acids(e.g. dsRNA, siRNA or sh RNA), polypeptide(e.g. soluble ligands, antibodies or antigen-binding fragments thereof, immuneoadhesins, fusion proteins, oligopeptides and other molecules) or compound. For example, antibody is selected from: human antibody, humanized antibody, chimeric antibody, multispecific antibody, monoclonal antibody and polyclonal antibody. For example, the antigen-binding fragment is selected from Fab, Fab′, F(ab′)₂, Fv, scFv, bispecific antibody, Fd, dAb, VHH, large antibody and complementarity determining region (CDR) fragments.

In certain embodiment, the antibody or antigen-binding fragment thereof comprises a heavy chain variable region (VH) comprising HCDR1, HCDR2 and HCDR3.

In certain embodiment, the antibody or antigen-binding fragment thereof further comprises or does not comprise a CH1 domain.

In certain embodiment, the antibody or antigen-binding fragment thereof further comprises or does not comprise CH2 and CH3 domains. For example, the antibody or antigen-binding fragment comprises VH-CH2—CH3 domain

In certain embodiment, the antibody or antigen-binding fragment thereof further comprises an antibody heavy chain constant region

In certain embodiment, the antibody heavy chain constant region is a human antibody heavy chain constant region. For example, the human antibody heavy chain constant region is selected from constant regions derived from the group consisting of IgG1, IgG2, IgG3, IgG4 and variants thereof.

In certain embodiment, the antibody or antigen-binding fragment thereof further comprises a light chain variable region (VL) comprising LCDR1, LCDR2, LCDR3.

For example, the antibody or antigen-binding fragment comprise a heavy chain variable region (VL) comprising HCDR1, HCDR2, HCDR3, and a light chain variable region (VL) comprising LCDR1, LCDR2, LCDR3.

In certain embodiment, the antibody or antigen-binding fragment comprises a light chain constant region (CL). For example, the antibody or antigen-binding fragment comprises a heavy chain comprising VH and CH1, and light chain comprising VL and CL. For example, the antibody or antigen-binding fragment comprises a heavy chain comprising VH, CH1, CH2 and CH3, and antibody light chain comprising VL and CL.

In certain embodiment, the checkpoint inhibitor could be PD-1/PD/L1 inhibitor. The PD-1/PD/L1 inhibitor comprise but not limited to those described in U.S. Pat. Nos. 7,488,802, 7,943,743, 8,008,449, 8,168,757, 8,217,149, 8,609,089, US 2010/028330, US 2012/0114649, WO 2003/042402, WO 2008/156712, WO 2010/089411, WO 2010/036959, WO 2011/066342, WO 2011/159877, WO 2011/082400 and WO 2011/161699 those described in, all of which are incorporated by reference into this application in their entirety.

In certain embodiment, checkpoint modulate could be PD-1 inhibitor. For example, PD-1 modulate could be anti-PD-1 antibody.

For example, the anti-PD-1 antibody can be Nivolumab. Alternative names for Nivolumab include MDX-1106, MDX-1106-04, ONO-4538, or BMS-936558, and the CAS registry number is: 946414-94-4. Nivolumab is a full-length human IgG4 monoclonal antibody that specifically blocks PD-1. Nivolumab (clone 5C4) and other human monoclonal antibodies that specifically bind PD-1 are disclosed in U.S. Pat. No. 8,008,449 and WO 2006/121168.

For example, the anti-PD-1 antibody can be Pembrolizumab. Pembrolizumab (trade name KEYTRUDA, formerly Lambrolizumab, also known as Merck 3745, MK-3475 or SCH-900475) is a humanized IgG4 monoclonal antibody that binds to PD-1. Pembrolizumab is disclosed, e.g., in Hamid, O. et al. (2013) New England Journal of Medicine 369(2):134-44, WO 2009/114335, and U.S. Pat. No. 8,354,509.

For example, the anti-PD-1 antibody can be Pidilizumab. Pidilizumab (CT-011; CureTech) is a humanized IgG1k monoclonal antibody that binds to PD-1. Pidilizumab and other humanized anti-PD-1 monoclonal antibodies are disclosed in WO 2009/101611. Other anti-PD1 antibodies are disclosed in U.S. Pat. No. 8,609,089, US 2010/028330 and/or US 2012/0114649.

For example, anti-PD1 antibody could be AMP-514(Amplimmune).

For example, the PD-1 inhibitor may be an immunoadhesin (e.g., an immunoadhesin comprising an extracellular or PD-1 binding portion of PDL1 or PDL2 fused to a constant region (e.g., an Fc region of an immunoglobulin sequence)).

In certain embodiment, checkpoint modulate is PD-L1 inhibitor. For example, checkpoint modulate could be anti PD-L1 antibody or its antigen-binding fragment. “PD-L1 antibody or antigen-binding fragment thereof” may include any anti-PD-L1 antibody or antigen-binding fragment thereof described in the art.

In certain embodiment, the anti-PD-L1 antibody or antigen-binding fragment thereof comprises a heavy chain variable region of an antibody, wherein the heavy chain variable region comprises HCDR1, HCDR2, HCDR3, which has at least about 80% (e.g. about 85%, 90%, or 95%) sequence identity to HCDR1, HCDR2, HCDR3, respectively, of atezolizumab, avelumab, BMS-936559, MPDL3280A (RG7446), or durvalumab (MEDI-4736).

In certain embodiment, the heavy chain is respectively associated with a heavy chain of the following molecule having at least about 80% (e.g. about 85%, 90%, or 95%) sequence identity: Atezolizumab, Avelumab, BMS-936559, MPDL3280A, or durvalumab.

In certain embodiment, the anti-PD-L1 antibody or antigen-binding fragment thereof comprises a heavy chain of an antibody, wherein the heavy chain has at least about 80% (e.g. about 85%, 90%, or 95%) sequence identity to the heavy chain, respectively, of atezolizumab, avelumab, BMS-936559, MPDL3280A (RG7446), or durvalumab (MEDI-4736).

In certain embodiment, the anti-PD-L1 antibody or antigen-binding fragment thereof further comprises an antibody light chain variable region, wherein the light chain variable region comprises LCDR1, LCDR2, LCDR3, each of which has at least about 80% (e.g. about 85%, 90%, or 95%) sequence identity to LCDR1, LCDR2, LCDR3, respectively, of atezolizumab, avelumab, BMS-936559, MPDL3280A, or durvalumab.

For example, the anti-PD-L1 antibody or antigen-binding fragment thereof comprises a heavy chain variable region and a light chain variable region of an antibody, wherein the heavy chain variable region comprises HCDR1, HCDR2, HCDR3, wherein the light chain can be The variable regions comprise LCDR1, LCDR2, LCDR3, each of which has at least about 80% (e.g., about 85%, 90%, or 95%) sequence identity to HCDR1, HCDR2, HCDR3, LCDR1, LCDR2, LCDR3, respectively, of: Atezolizumab, Avelumab, BMS-936559, MPDL3280A (RG7446), or durvalumab (MEDI4736).

In certain embodiment, the light chain variable region have at least about 80% (e.g., about 85%, 90%, or 95%) sequence identity to the light chain variable region of atezolizumab, avelumab, BMS-936559, MPDL3280A, or durvalumab, respectively.

For example, the anti-PD-L1 antibody or antigen-binding fragment thereof comprises a heavy chain variable region and a light chain variable region, wherein is respectively associated with the heavy chain variable region and the light chain variable region of the following molecule having at least about 80% (e.g., about 85%, 90%, or 95%) sequence identity: Atezolizumab, Avelumab, BMS-936559, MPDL3280A (RG7446) or durvalumab (RG7446).

In certain embodiment, the anti-PD-L1 antibody or antigen-binding fragment thereof comprises a light chain, wherein the light chain having at least about 80% (e.g., about 85%, 90%, or 95%) sequence identity: Atezolizumab, Avelumab, BMS-936559, MPDL3280A or durvalumab.

For example, the anti-PD-L1 antibody or antigen-binding fragment thereof comprises a heavy chain and a light chain, wherein is respectively associated with the heavy chain and the light chain of the following molecule having at least about 80% (e.g., about 85%, 90%, or 95%) sequence identity: Atezolizumab, Avelumab, BMS-936559, MPDL3280A (RG7446) or durvalumab (RG7446).

In certain embodiment, the anti-PD-L1 antibody or antigen-binding fragment thereof comprises: Atezolizumab, Avelumab, BMS-936559, MPDL3280A or durvalumab, or an antigen-binding fragment thereof, or a variant or biosimilar thereof, or a combination thereof

In certain embodiment, the anti-PD-L1 antibody can also be a commercially available or published PD-L1 antibody. Including but not limited to, such as PD-L1 antibody BMS-936559, MPDL3280A, MEDI4736, MSB0010718C (see US2014341917, US20130034559, U.S. Pat. No. 8,779,108) and so on.

For example, an anti-PD-L1 antibody is MDX-1105. MDX-1105, also known as BMS-936559, an anti-PD-L1 antibody as described in WO 2007/005874. For example, the anti-PD-L1 antibody is YW243.55.S70. The YW243.55.S70 antibody is an anti-PD-L1 antibody as described in WO 2010/077634. The heavy and light chain variable region sequences of YW243.55.S70 are also described in WO2010/077634. For example, the anti-PD-L1 antibody is MDPL3280A (Genentech/Roche). MDPL3280A is a human Fc-optimized IgG1 monoclonal antibody that binds to PD-L1. MDPL3280A and other human monoclonal antibodies to PD-L1 are described in U.S. Pat. No. 7,943,743 and US2012/0039906. For example, an anti-PD-L1 antibody is Avelumab (MSB0010718C). MSB0010718C (also known as A09-246-2; Merck Serono) is a monoclonal antibody that binds to PD-L1. Other humanized anti-PD-L1 antibodies are disclosed in WO 2013/079174. For example, an anti-PD-L1 antibody is durvalumab (CAS Number: 1428935-60-7). For example, the anti-PD-L1 antibody is Atezolizumab (CAS Number: 1380723-44-3).

In certain embodiment, the anti-PD-L1 antibody or antigen-binding fragment thereof comprises a heavy chain variable region of an antibody, wherein the heavy chain variable region comprises one or more heavy chain variable region CDRs (HCDRs) selected from the group consisting of: (a) HCDR1 having at least about 70% sequence identity to the amino acid sequence shown in SEQ ID NO:1; (b) having at least about 70% sequence identity to SEQ ID NO:2 HCDR2 of sequence identity; and (c) HCDR3 having at least about 70% sequence identity to SEQ ID NO:3.

In certain embodiment, the anti-PD-L1 antibody or antigen-binding fragment thereof comprises an antibody heavy chain variable region, wherein the heavy chain variable region comprises one or more HCDRs selected from the group consisting of: (a) HCDR1 having the amino acid sequence shown in SEQ ID NO: 1 or having no more than 2 amino acid differences from the amino acid sequence shown in SEQ ID NO: 1 obtained through amino acid addition, elimination or substitution reactions HCDR1; (b) HCDR2 having the amino acid sequence shown in SEQ ID NO: 2 or HCDR2 having no more than 2 amino acid differences from the amino acid sequence shown in SEQ ID NO: 2 obtained through amino acid addition, elimination or substitution reactions; and (c) HCDR3 having the amino acid sequence shown in SEQ ID NO: 3 or HCDR3 having no more than 2 amino acid differences from the amino acid sequence shown in SEQ ID NO: 3 obtained through amino acid addition, elimination or substitution reactions.

In certain embodiment, the anti-PD-L1 antibody or antigen-binding fragment thereof comprises a heavy chain variable region of an antibody, wherein the heavy chain variable region comprises HCDR1, HCDR2, HCDR3, the said HCDR1 comprises an amino acid sequence with at least about 70% sequence identity to the amino acid sequence shown in SEQ ID NO: 1, said HCDR2 comprises an amino acid sequence with at least about 70% sequence identity to the amino acid sequence shown in SEQ ID NO: 2, wherein the HCDR3 comprises an amino acid sequence having at least about 70% sequence identity to the amino acid sequence set forth in SEQ ID NO:3.

In certain embodiment, the anti-PD-L1 antibody or antigen-binding fragment thereof comprises a heavy chain variable region of an antibody, wherein the heavy chain variable region comprises HCDR1, HCDR2, HCDR3, the said HCDR1 comprises the amino acid sequence shown in SEQ ID NO: 1 or an amino acid sequence obtained by amino acid addition, elimination or substitution reaction with no more than 2 amino acid differences from the amino acid sequence shown in SEQ ID NO: 1; HCDR2 comprises the amino acid sequence shown in SEQ ID NO: 2 or an amino acid sequence obtained by amino acid addition, elimination or substitution reaction with no more than 2 amino acid differences from the amino acid sequence shown in SEQ ID NO: 2; the HCDR3 comprising the amino acid sequence shown in SEQ ID NO: 3 or an amino acid sequence obtained by amino acid addition, elimination or substitution reaction and having no more than 2 amino acid differences from the amino acid sequence shown in SEQ ID NO: 3.

In certain embodiment, the anti-PD-L1 antibody or antigen-binding fragment thereof comprises an antibody heavy chain variable region, wherein the heavy chain variable region comprises an amino acid sequence selected from the group consisting of: (a) the amino acid sequence set forth in SEQ ID NO: 4; (b) an amino acid sequence at least about 85%, 90%, 95% or 99% sequence identity to the amino acid sequence set forth in SEQ ID NO: 4; and (c) an amino acid sequence having 1 or more differences from the amino acid sequence shown in SEQ ID NO: 4 obtained by addition, elimination or substitution reaction.

In certain embodiment, the TGFβ inhibitor binds to TGFβ1, TGFβ2 or TGFβ3.

In certain embodiment, the TGFβ inhibitor binds to TGFβ or transforming growth factor β receptor (TGFβR).

For example, the TGFβ inhibitor binds to TGFβ type I receptor (TGFβRI), TGFβ type II receptor (TGFβRII) or TGFβ type III receptor (TGFβRIII).

In certain embodiment, the TGFβ inhibitor comprises: 1) an anti-TGFβ antibody or an antigen-binding fragment thereof; 2) an anti-TGFβR antibody or an antigen-binding fragment thereof; 3) small molecule TGFβ inhibitors; 4) proteins comprising transforming growth factor β receptor II (TGFβRII) or functionally active fragments thereof, or variants or biosimilars thereof, or combinations thereof.

In certain embodiment, anti-TGFβ antibody or antigen-binding fragment thereof comprises an antibody heavy chain variable region, wherein said heavy chain variable region comprises HCDR1, HCDR2, HCDR3, each of which has at least about 80% (e.g. about 85%, 90%, or 95%) sequence identity with HCDR1, HCDR2, and HCDR3 of the fresolimumab molecule (CAS Number: 948564-73-6)).

In certain embodiment, the heavy chain variable region has at least about 80% (e.g. about 85%, 90%, or 95%) sequence identity to the heavy chain variable region of Fresolimumab.

In certain embodiment, the heavy chain has at least about 80% (e.g. about 85%, 90%, or 95%) sequence identity to the heavy chain variable region of Fresolimumab.

In certain embodiment, the anti-TGFβ antibody or antigen-binding fragment thereof comprises an antibody's light chain variable region, wherein said light chain variable region comprises LCDR1, LCDR2, LCDR3, each of which has at least about 80% (e.g. about 85%, 90%, or 95%) sequence identity with LCDR1, LCDR2, and LCDR3 of the Fresolimumab.

For example, the anti-TGFβ antibody or antigen-binding fragment thereof comprises a heavy chain variable region and a light chain variable region of an antibody, wherein the heavy chain variable region comprises HCDR1, HCDR2, HCDR3, wherein the light chain variable region comprises LCDR1, LCDR2, LCDR3, each of which may have at least about 80% (eg, 85%, 90%, or 95%) sequence identity with HCDR1, HCDR2, HCDR3, LCDR1, LCDR2, LCDR3 of the Fresolimumab molecule, respectively.

In certain embodiment, the light chain variable region has at least about 80% sequence identity to the light chain variable region.

For example, the anti-TGFβ antibody or antigen-binding fragment thereof comprises a heavy chain variable region and a light chain variable region of an antibody, each of which can be at least about (eg, 85%, 90%, or 95%) sequence identity with such heavy chain variable region and light chain variable region of Fresolimumab molecule.

In certain embodiment, the light chain has at least about 80% sequence identity to the light chain variable region.

For example, the anti-TGFβ antibody or antigen-binding fragment thereof comprises a heavy chain and a light chain of the antibody, each of which may have at least about 80% (e.g., 85%, 90% or 95%) sequence identity with the heavy chain and light chain of the Fresolimumab molecule, respectively.

In certain embodiment, the anti-TGFβ antibody or antigen-binding fragment thereof comprises: Fresolimumab, or an antigen-binding fragment thereof, or a variant or biosimiliar thereof.

In certain embodiment, the small molecule TGFβ inhibitor comprises the following compound: SB525334, SD-208, SB431542, LY2109761, LY2157299, GW788388, RepSox, SIS3, LDN-193189, EW-7197, LY364947, or a combination thereof.

In certain embodiment, the TGFβRII comprises a polypeptide having a wild-type human TGFβ receptor type 2 isoform A sequence (For example, the amino acid sequence of NCBI reference sequence (Ref Seq) accession number NP_001020018), or a polypeptide having a wild-type human TGFβ receptor type 2 isoform B sequence(For example, the amino acid sequence of NCBI reference sequence (Ref Seq) accession number NP_003233), or a polypeptide having a sequence substantially identical to an amino acid sequence thereof.

In certain embodiment, wherein said TGFβ RII or its functionally active fragment comprises: human TGFβRII extracellular domain (ECD), any part of NCBI Ref Seq NO. NP_001020018 or NCBI Ref Seq NO. NP_003233, or an amino acid sequence substantially identical to sequence ((e.g., about 80%, 85%, 90% or 95%)).

In certain embodiment, wherein the TGFβRII or a functionally active fragment thereof comprises:
(a) the amino acid sequence of SEQ ID NO: 6;
(b) an amino acid sequence having at least about 85% sequence identity to the amino acid sequence set forth in SEQ ID NO: 6; Or
(c) has an amino acid sequence in which one or more amino acids are added, deleted and/or substituted as compared to the amino acid sequence shown in SEQ ID NO: 6.

In certain embodiment, wherein the bifunctional immune checkpoint/TGF β inhibitor is a fusion protein.

In certain embodiment, wherein the PD-L1/TGFβ dual inhibitor or PD-1/TGFβ dual inhibitor is a fusion protein.

In certain embodiment, wherein the PD-L1//TGF β dual inhibitor comprises a PD-L1 targeting moiety and a TGF β receptor domain comprising transforming growth factor β receptor II (TGFβ RII) or a functionally active fragment thereof.

In certain embodiment, wherein the PD-L1//TGF β dual inhibitor comprises a polypeptide, wherein the polypeptide comprises at least: (i) a heavy chain variable region of an anti-PD-L1antibody; And (ii) TGFβII or a functionally active fragment thereof.

In certain embodiment, wherein the anti-PD-L1 antibody heavy chain variable region comprises HCDR1, HCDR2, HCDR3; Wherein the HCDR1 comprises an amino acid sequence having at least about 70% sequence identity to the amino acid sequence set forth in SEQ ID NO: 1, the HCDR2 comprises an amino acid sequence having at least about 70% sequence identity to the amino acid sequence set forth in SEQ ID NO: 2, and the HCDR3 comprises an amino acid sequence having at least about 70% sequence identity to the amino acid sequence set forth in SEQ ID NO: 3.

In certain embodiment, wherein the anti-PD-L1 antibody heavy chain variable region comprises HCDR1, HCDR2, HCDR3, and the HCDR1 comprises the amino acid sequence shown in SEQ ID NO: 1 or an amino acid sequence obtained by amino acid addition, elimination or substitution reaction and having no more than 2 amino acid differences from the amino acid sequence shown in SEQ ID NO: 1; The HCDR2 comprises the amino acid sequence represented by SEQ ID NO: 2 or an amino acid sequence obtained by amino acid addition, elimination or substitution reaction and having no more than 2 amino acid differences from the amino acid sequence represented by SEQ ID NO: 2; The HCDR3 comprises an amino acid sequence represented by SEQ ID NO: 3 or an amino acid sequence obtained by an amino acid addition, elimination or substitution reaction and having no more than 2 amino acid differences from the amino acid sequence represented by SEQ ID NO: 3.

In certain embodiment, wherein the anti-PD-L1 antibody heavy chain variable region comprises an amino acid sequence selected from the group consisting of: (a) the amino acid sequence set forth in SEQ ID NO: 4; (b) an amino acid sequence having at least about 85%, 90%, 95%, or 99% sequence identity to the amino acid sequence set forth in SEQ ID NO: 4; (c) an amino acid sequence having 1 or more differences from the amino acid sequence shown in SEQ ID NO: 4 obtained by addition, elimination or substitution.

In certain embodiment, wherein the TGFβRII or a functionally active fragment thereof comprises:
(a) the amino acid sequence of SEQ ID NO: 6;
(b) an amino acid sequence having at least about 85% sequence identity to the amino acid sequence set forth in SEQ ID NO: 6; or
(c) an amino acid sequence in which one or more amino acids are added, deleted and/or substituted as compared to the amino acid sequence shown in SEQ ID NO: 6

In certain embodiment, wherein the polypeptide further comprises a linker that links the C-terminus of the heavy chain variable region of the anti-PD-L1 antibody or antigen-binding fragment thereof to the N-terminus of the TGFβRII or functionally active fragment thereof.

In certain embodiment, wherein the polypeptide further comprising CH2, CH3 domains, said polypeptide comprising the heavy chain variable region (VH) of anti-PD-L1 antibody, CH2, CH3 domains and TGF βRR or a functionally active fragment thereof in sequence from N-terminus to C-terminus, said CH3 domain having its C-terminus connected to the N-terminus of said TGF βRII or a functionally active fragment thereof by a linker.

In certain embodiment, wherein the CH2, CH3 domains are derived from IgG.

In certain embodiment, wherein the linker is a peptide linker.

In certain embodiment, wherein the amino acid sequence of the peptide linker is (G₄S)ₓ, wherein x is any integer from 3 to 6.

In certain embodiment, wherein the peptide linker comprises an amino acid sequence selected from the group consisting of: (a) the amino acid sequence set forth in SEQ ID NO: 5; (b) an amino acid sequence having at least about 85%, 90%, 95%, or 99% sequence identity to the amino acid sequence set forth in SEQ ID NO: 5; And (c) an amino acid sequence having 1 or more differences from the amino acid sequence shown in SEQ ID NO: 5 obtained by addition, elimination or substitution reaction.

In certain embodiment, wherein the polypeptide comprises an amino acid sequence selected from the group consisting of: (a) the amino acid sequence set forth in SEQ ID NO: 7; (b) an amino acid sequence having at least about 85%, 90%, 95%, or 99% sequence identity to the amino acid sequence set forth in SEQ ID NO: 7; (c) an amino acid sequence having 1 or more differences from the amino acid sequence shown in SEQ ID NO: 7 obtained by addition, elimination or substitution reactions.

In certain embodiment, wherein the PD-L1//TGFβ dual inhibitor comprises two polypeptides aforementioned.

In certain embodiment, wherein the PD-L1/TGFβ dual inhibitor comprises a first polypeptide and a second polypeptide, wherein the first polypeptide comprises at least: (i) a heavy chain of an anti-PD-L1 antibody variable region; and (ii) TGFβRII or a functionally active fragment thereof; wherein the second polypeptide comprises at least the light chain variable region of an anti-PD-L1 antibody; wherein the heavy chain variable region of the first polypeptide and the light chain variable regions of the second polypeptide are capable of specifically binding to PD-L1.

In certain embodiment, wherein the first polypeptide further comprises a linker, said linker connects the C-terminus of the heavy chain variable region of the anti-PD-L1 antibody or antigen-binding fragment thereof with the TGFβRII or the N-terminus of its functionally active fragments.

In certain embodiment, wherein the PD-L1/TGFβ dual inhibitor comprises a first polypeptide and a second polypeptide, wherein, the first polypeptide comprises the heavy chain variable region (VH) of the anti-PD-L1 antibody, the CH1 domain, and TGFβRII or its functionally active fragment sequentially from the N-terminus to the C-terminus, and the C-terminus of the CH1 domain is connected to the TGFβRII or its functionally active fragment N-terminus is connected by a linker; and the second polypeptide comprises a light chain variable region (VL) and a light chain constant region (CL) of an anti-PD-L1 antibody from the N-terminus to the C-terminus).

In certain embodiment, wherein the PD-L1/TGFβ dual inhibitor comprises a first polypeptide and a second polypeptide, wherein, the first polypeptide comprises the heavy chain variable region (VH) of the anti-PD-L1 antibody, the CH1, CH2, CH3 domain, and TGFβRII or its functionally active fragment sequentially from the N-terminus to the C-terminus, and the C-terminus of the CH3 domain is connected to the TGFβRII or its functionally active fragment N-terminus is connected by a linker; and the second polypeptide comprises a light chain variable region (VL) and a light chain constant region (CL) of an anti-PD-L1 antibody from the N-terminus to the C-terminus).

In certain embodiment, wherein said linker is a peptide linker.

In certain embodiment, wherein the first polypeptide has at least about 80% sequence identity to a polypeptide having the same function as: M7824 or SHR-1701.

In certain embodiment, wherein the second polypeptide has at least about 80% sequence identity to a polypeptide having the same function as: M7824 or SHR-1701.

In certain embodiment, wherein the PD-L1/TGFβ dual inhibitor comprises M7824, SHR-1702, or variant or biosimilar or their combination.

In certain embodiment, a PI3K inhibitor can be a compound that inhibits one or more PI3K isoforms, e.g., α, β, δ, or γ isoforms. In one embodiment, the PI3K inhibitor may be a compound that inhibits the α, β, δ, or γ isoforms of PI3K. In another embodiment, the PI3K inhibitor may be a compound that inhibits the β, δ, or γ isoforms of PI3K. In another embodiment, the PI3K inhibitor may be a compound that inhibits the δ and γ isoforms of PI3K, ie, a PI3K δ/γ dual inhibitor.

In certain embodiment, wherein the said PI3K inhibitors comprise compounds as formula (IA-I), (IA-II), (IA-III), (IA-IV):

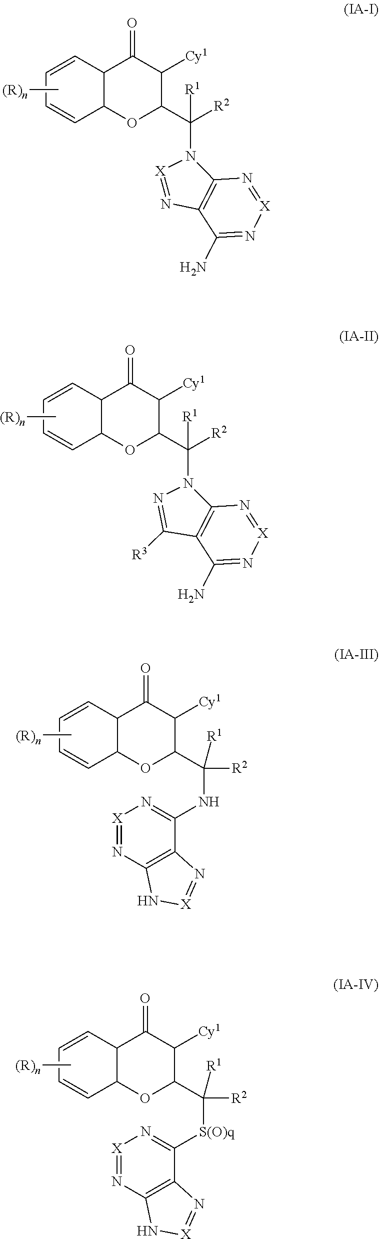

or their enantiomers, mixtures of enantiomers, mixtures of two or more diastereoisomers, isotopic variants, pharmaceutically acceptable salts, solvates, hydrates or prodrug, wherein,
each occurrence of R is independently selected from hydrogen, halogen, —OR^{a}, CN, substituted or unsubstituted C₁₋₆ alkyl, substituted or unsubstituted C₂₋₆ alkenyl, substituted or unsubstituted C₂₋₆ alkynyl, substituted or unsubstituted C₃₋₈ cycloalkyl, and substituted or unsubstituted heterocyclic group;
R¹ and R² may be the same or different and are independently selected from hydrogen, halogen, and substituted or unsubstituted C₁₋₆ alkyl, or both R¹ and R² directly bound to a common atom, may be joined to form an oxo group (═O) or a substituted or unsubstituted saturated or unsaturated 3-10 member ring, including the carbon atom to which R¹ and R² are bound, which may optionally include one or more heteroatoms which may be the same or different and are selected from O, NR^{a} and S;
Cy¹ is selected from substituted or unsubstituted C₃₋₈ cycloalkyl, substituted or unsubstituted 3 to 15 membered heterocyclic groups having at least one heteroatom selected from N, O and S, substituted or unsubstituted C₆₋₂₀ aryl and substituted or unsubstituted 5 to 14 membered heteroaryl monocyclic groups with one or more heteroatoms selected from N, O and S;
each occurrence of R^{a} may be the same or different and are independently selected from hydrogen, halogen, hydroxy, cyano, substituted or unsubstituted C₁₋₆alkyl, —NR^{c}R^{d}, wherein RC and R d are independently hydrogen, halogen, hydroxy, cyano, substituted or unsubstituted C¹⁻⁶alkyl, and C₁₋₆alkoxy, and —OR^{c}, wherein R^{c} is substituted or unsubstituted C₁₋₆alkyl,
n is an integer from 1 to 4;
q is 0, 1 or 2;
each occurrence of X is independently selected from CR³ or N;
and each occurrence of R³ is independently selected from hydrogen, hydroxy, halogen, carboxyl, cyano, nitro, substituted or unsubstituted C₁₋₈ alkyl, substituted or unsubstituted C₁₋₈ alkoxy, substituted or unsubstituted C₂₋₁₀ alkenyl, substituted or unsubstituted C₂₋₁₀ alkynyl, substituted or unsubstituted C₆₋₂₀ aryl, substituted or unsubstituted C₆₋₂₀ aryl C₁₋₈alkyl, substituted or unsubstituted C₃₋₂₀ cycloalkyl, substituted or unsubstituted C₃₋₂₀ cycloalkyl C₁₋₈ alkyl, substituted or unsubstituted C₃₋₈ cycloalkenyl C₁₋₈ alkyl, substituted or unsubstituted C₃₋₈ cycloalkene, substituted or unsubstituted 5-14-membered heteroaryl with one or more heteroatoms selected from N, O and S, substituted or unsubstituted with one or more heteroatoms selected from N, O, S 5-14 membered heteroaryl C₁₋₈ alkyl, substituted or unsubstituted 3 to 15 membered heterocycles having at least one heteroatom selected from N, O and S, substituted or unsubstituted 3 to 15 membered heterocyclyl C₁₋₈ alkyl rings having at least one heteroatom selected from N, O and S, substituted or unsubstituted guanidine, —COOR^{x}, —C(O)R^{x}, —C(S)R^{x}, —C(O)NR^{x}R^{y}, —C(O)ONR^{x}R^{y}, —NR^{y}R^{z}, —NR^{x}CONR^{y}R^{z}, —N(R^{x})SOR^{y}, —N(R^{x})SO₂R^{y}, —(═N—N(R^{x})R^{y}), —NR^{x}C(O)OR^{y}, —NR^{x}R^{y}, —NR^{x}C(O)R^{y}—, —NR^{x}C(S)R^{y}—NR^{x}C(S)NR^{y}R^{z}, —SONR^{x}R^{y}—, —S0₂NR^{x}R^{y}—, —OR^{x}, —OR^{x}C(O)NR^{y}R^{z}, —OR^{x}C(O)OR^{y}—, —OC(O)R^{x}, —OC(O)NR^{x}R^{y}, —R^{x}NR^{y}C(O)R^{z}, —R^{x}OR^{y}, —R^{x}C(O)OR^{y}, —R^{x}C(O)NR^{y}R^{z}, —R^{x}C(O)R^{x}, —R^{x}OC(O)R^{y}, —SR^{x}, —SOR^{x}, —SO₂R^{x}, —ONO₂,
wherein R^{x}, R^{y} and R^{z} in each of the above groups can be hydrogen, substituted or unsubstituted C₁₋₈ alkyl, substituted or unsubstituted C₁₋₈ alkoxy, substituted or unsubstituted C₂₋₁₀ alkenyl, substituted or unsubstituted C₂₋₁₂ alkynyl, substituted or unsubstituted C₆₋₂₀ aryl, substituted or unsubstituted C₆₋₂₀ aryl C₁₋₈ alkyl , substituted or unsubstituted C₃₋₂₀ cycloalkyl, substituted or unsubstituted C₃₋₂₀ cycloalkylC₁₋₈ alkyl, substituted or unsubstituted C₃₋₈ cycloalkenyl, substituted or unsubstituted 5 to 14 membered heteroaryl with one or more heteroatoms selected from N, O and S, substituted or unsubstituted heteroaryl with one or more heteroatoms selected from N, O and S atomic 5 to 14 membered heteroaryl C₁₋₈ alkyl, substituted or unsubstituted 3 to 15 membered heterocycle having at least one heteroatom selected from N, O and S, substituted or unsubstituted 3 to 15 membered heterocyclyl C₁₋₈ alkyl ring having at least one heteroatom selected from N, O and S or substituted or unsubstituted amino, or any two of R^{x}, R^{y} and R^{z} may be joined to form a substituted or unsubstituted saturated or unsaturated 3-10 membered ring, which may optionally include heteroatoms which may be the same or different and are selected from O, NR^{x} or S, wherein R^{x} is hydrogen or substituted or unsubstituted alkyl.

In certain embodiment, the term “substituted” generally refers to substitution with any one or any combination of the following substituents, which may be the same or different and independently selected from the following substituents: hydrogen, hydroxyl, halogen, carboxyl, cyano, nitro, ═O group, ═S group, C₁-C₈ alkyl, C₁-C₈ alkoxy, C₂-C₁₀ alkenyl, C₂-C₁₂ alkynyl, C₆-C₂₀ aryl, C₆-C₂₀ aryl (C₁-C₈ alkyl), C₃-C₁₂ cycloalkyl, C₃-C₁₂ cycloalkyl (C₁-C₈ alkyl), C₃-C₈ cycloalkenyl, C₃-C₈ cycloalkenyl (C₁-C₈ alkyl); 5-14 membered heteroaryl A group, wherein the heteroatom is selected from N, O and S; 5-14 membered heteroaryl (C₁-C₈ alkyl), wherein the heteroatom is selected from N, O and S; 3-15 membered heterocyclic ring, Wherein the heteroatom is selected from N, O and S; and 3-15 membered heterocyclic group (C₁-C₈ alkyl), wherein the heteroatom is selected from N, O and S.

In certain embodiment, guanidine, —COOR^{x}, —C(O)R^{x}, —C(S)R^{x}, —C(O)NR^{x}R^{y}, —C(O)ONR^{x}R^{y}, —NR^{y}R^{z}, —NR^{x}CONR^{y}R^{z}, —N(R^{x})SOR^{y}, —N(R^{x})SO₂R^{y}, —(═N—N(R^{x})R^{y}), —NR^{x}C(O)OR^{y}, —NR^{x}R^{y}, —NR^{x}C(O)R^{y}—, —NR^{x}C(S)R^{y}, —NR^{x}C(S) NR^{y}R^{z}, —SONR^{x}R^{y}—, SO₂NR^{x}R^{y}—, —OR^{x}, —OR^{x}C(O)NR^{y}R^{z}, —OR^{x}C(O)OR^{y}—, —OC(O)R^{x}, —OC(O)NR^{x}R^{y}, —R^{x}NR^{y}C(O)R^{z}, —R^{x}OR^{y}, —R^{x}C(O)OR^{y}, —R^{x}C(O)NR^{y}R^{z}, —R^{x}C(O)R^{x}, —R^{x}OC(O)R^{x}, —SR^{x}, —SOR^{x}, —SO₂R^{x} and —ONO₂, wherein each of the above groups R^{x}, R^{y} and R^{z} can be hydrogen, C₁-C₈ alkyl, C₁-C₈ alkoxy, C₂-C₁₀ alkenyl, C₂-C₁₂ alkynyl, C₆-C₂₀ aryl, C₆-C₂₀aryl (C₁-C₈ alkyl), C₃-C₁₂ cycloalkyl, C₃-C₁₂ cycloalkyl (C₁-C₈alkyl), C₃-C₈ cycloalkenyl, amino; 5-14 membered heteroaryl, wherein the heteroatom is selected from N, O and S; 5-14 membered heteroaryl (C₁-C₈ alkyl), wherein said heteroatoms are selected from N, O and S; 3-15 membered heterocyclic rings, wherein said heteroatoms are selected from N, O and S; 3-15 membered heterocyclic group (C₁-C₈ alkyl), or any two of R^{x}, ^{Ry} and R^{z} can be connected to form a saturated or unsaturated 3-10 membered ring, which can optionally include the same or different heteroatoms selected from O, NR^{x} or S, wherein R^{x} is hydrogen or C₁₋₆ alkyl.

In certain embodiment, wherein the PI3K inhibitor comprises compounds as formula (IA-V):

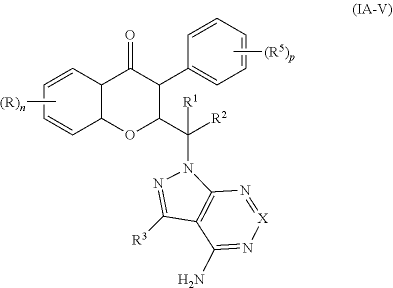

or their enantiomers, mixtures of enantiomers, mixtures of two or more diastereoisomers, isotopic variants, pharmaceutically acceptable salts, solvates, hydrates or prodrug, wherein
each occurrence of R is independently selected from hydrogen, halogen, —OR^{a}, CN, substituted or unsubstituted C₁₋₆ alkyl, substituted or unsubstituted C₂₋₆ alkenyl, substituted or unsubstituted C₂₋₆ alkynyl, substituted or unsubstituted C₃₋₈ cycloalkyl, and substituted or unsubstituted heterocyclic group;
R¹ and R² may be the same or different and are independently selected from hydrogen, halogen, and substituted or unsubstituted C₁₋₆ alkyl, or both R¹ and R² directly bound to a common atom, may be joined to form an oxo group (═O) or a substituted or unsubstituted saturated or unsaturated 3-10 member ring, including the carbon atom to which R¹ and R² are bound, which may optionally include one or more heteroatoms which may be the same or different and are selected from O, NR^{a} and S;
each occurrence of X is independently selected from CR³ or N; and
each occurrence of R³ is independently selected from hydrogen, hydroxy, halogen, carboxyl, cyano, nitro, substituted or unsubstituted C₁₋₈ alkyl, substituted or unsubstituted C₁₋₈ alkoxy, substituted or unsubstituted C₂₋₁₀ alkenyl, substituted or unsubstituted C₂₋₁₀ alkynyl, substituted or unsubstituted C₆₋₂₀ aryl, substituted or unsubstituted C₆₋₂₀ aryl C₁₋₈alkyl, substituted or unsubstituted C₃₋₂₀ cycloalkyl, substituted or unsubstituted C₃₋₂₀ cycloalkyl C₁₋₈ alkyl, substituted or unsubstituted C₃₋₈ cycloalkenyl C₁₋₈ alkyl, substituted or unsubstituted C₃₋₈ cycloalkene, substituted or unsubstituted 5-14-membered heteroaryl with one or more heteroatoms selected from N, O and S, substituted or unsubstituted with one or more heteroatoms selected from N, O, S 5-14 membered heteroaryl C₁₋₈ alkyl, substituted or unsubstituted 3 to 15 membered heterocycles having at least one heteroatom selected from N, O and S, substituted or unsubstituted 3 to 15 membered heterocyclyl C₁₋₈ alkyl rings having at least one heteroatom selected from N, O and S, substituted or unsubstituted guanidine, —COOR^{x}, —C(O)R^{x}, —C(S)R^{x}, —C(O)NR^{x}R^{y}, —C(O)ONR^{x}R^{y}, —NR^{y}R^{z}, —NR^{x}CONR^{y}R^{z}, —N(R^{x})SOR^{y}, —N(R^{x})SO₂R^{y}, —(═N—N(R^{x})R^{y}), —NR^{x}C(O)OR^{y}, —NR^{x}R^{y}, —NR^{x}C(O)R^{y}—, —NR^{x}C(S)R^{y}—NR^{x}C(S)NR^{y}R^{z}, —SONR^{x}R^{y}—, —S0₂NR^{x}R^{y}—, —OR^{x}, —OR^{x}C(O)NR^{y}R^{z}, —OR^{x}C(O)OR^{y}—, —OC(O)R^{x}, —OC(O)NR^{x}R^{y}, —R^{x}NR^{y}C(O)R^{z}, —R^{x}OR^{y}, —R^{x}C(O)OR^{y}, —R^{x}C(O)NR^{y}R^{z}, —R^{x}C(O)R^{x}, —R^{x}OC(O)R^{y}, —SR^{x}, —SOR^{x}, —SO₂R^{x}, —ONO₂,
wherein R^{x}, R^{y} and R^{z} in each of the above groups can be hydrogen, substituted or unsubstituted C₁₋₈ alkyl, substituted or unsubstituted C₁₋₈ alkoxy, substituted or unsubstituted C₂₋₁₀ alkenyl, substituted or unsubstituted C₂₋₁₂ alkynyl, substituted or unsubstituted C₆₋₂₀ aryl, substituted or unsubstituted C₆₋₂₀ aryl C₁₋₈ alkyl, substituted or unsubstituted C₃₋₂₀ cycloalkyl, substituted or unsubstituted C₃₋₂₀ cycloalkylC₁₋₈ alkyl, substituted or unsubstituted C₃₋₈ cycloalkenyl, substituted or unsubstituted 5 to 14 membered heteroaryl with one or more heteroatoms selected from N, O and S, substituted or unsubstituted heteroaryl with one or more heteroatoms selected from N, O and S atomic 5 to 14 membered heteroaryl C₁₋₈ alkyl, substituted or unsubstituted 3 to 15 membered heterocycle having at least one heteroatom selected from N, O and S, substituted or unsubstituted 3 to 15 membered heterocyclyl C₁₋₈ alkyl ring having at least one heteroatom selected from N, O and S or substituted or unsubstituted amino, or any two of R^{x}, R^{y} and R^{z} may be joined to form a substituted or unsubstituted saturated or unsaturated 3-10 membered ring, which may optionally include heteroatoms which may be the same or different and are selected from O, NR^{x} or S, wherein R^{x} is hydrogen or substituted or unsubstituted alkyl,
each occurrence of R⁵ is hydrogen, C₁₋₆ alkyl or halogen,
n is 0, 1, 2, 3 or 4; and
P is 0, 1, 2, 3, 4 or 5.

In certain embodiment, wherein the PI3K inhibitor comprises compounds as formula (IA-VI):

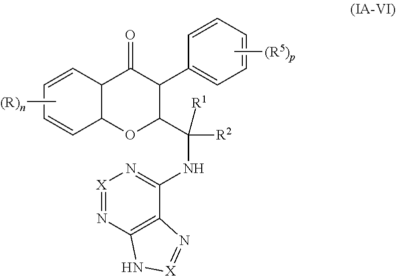

or their enantiomers, mixtures of enantiomers, mixtures of two or more diastereoisomers, isotopic variants, pharmaceutically acceptable salts, solvates, hydrates or prodrug, wherein
each occurrence of R is independently selected from hydrogen, halogen, —OR^{a}, CN, substituted or unsubstituted C₁₋₆ alkyl, substituted or unsubstituted C₂₋₆ alkenyl, substituted or unsubstituted C₂₋₆ alkynyl, substituted or unsubstituted C₃₋₈ cycloalkyl, and substituted or unsubstituted heterocyclic group;
R¹ and R² may be the same or different and are independently selected from hydrogen, halogen, and substituted or unsubstituted C₁₋₆ alkyl, or both R¹ and R² directly bound to a common atom, may be joined to form an oxo group (=0) or a substituted or unsubstituted saturated or unsaturated 3-10 member ring, including the carbon atom to which R¹ and R² are bound, which may optionally include one or more heteroatoms which may be the same or different and are selected from O, NR^{a} and S;
each occurrence of X is independently selected from CR³ or N; and
each occurrence of R³ is independently selected from hydrogen, hydroxy, halogen, carboxyl, cyano, nitro, substituted or unsubstituted C₁₋₈ alkyl, substituted or unsubstituted C₁₋₈ alkoxy, substituted or unsubstituted C₂₋₁₀ alkenyl, substituted or unsubstituted C₂₋₁₀ alkynyl, substituted or unsubstituted C₆₋₂₀ aryl, substituted or unsubstituted C₆₋₂₀ aryl C₁₋₈ alkyl, substituted or unsubstituted C₃₋₂₀ cycloalkyl, substituted or unsubstituted C₃₋₂₀ cycloalkyl C₁₋₈ alkyl, substituted or unsubstituted C₃₋₈ cycloalkenyl C₁₋₈ alkyl, substituted or unsubstituted C₃₋₈ cycloalkene, substituted or unsubstituted 5-14-membered heteroaryl with one or more heteroatoms selected from N, O and S, substituted or unsubstituted with one or more heteroatoms selected from N, O, S 5-14 membered heteroaryl C₁₋₈ alkyl, substituted or unsubstituted 3 to 15 membered heterocycles having at least one heteroatom selected from N, O and S, substituted or unsubstituted 3 to 15 membered heterocyclyl C₁₋₈ alkyl rings having at least one heteroatom selected from N, O and S, substituted or unsubstituted guanidine, —COOR^{x}, —C(O)R^{x}, —C(S)R^{x}, —C(O)NR^{x}R^{y}, —C(O)ONR^{x}R^{y}, —NR^{y}R^{z}, —NR^{x}CONR^{y}R^{z}, —N(R^{x})SOR^{y}, —N(R^{x})SO₂R^{y}, —(═N—N(R^{x})R^{y}), —NR^{x}C(O)OR^{y}, —NR^{x}R^{y}, —NR^{x}C(O)R^{y}—, —NR^{x}C(S)R^{y}—NR^{x}C(S)NR^{y}R^{z}, —SONR^{x}R^{y}—, —S0₂NR^{x}R^{y}—, —OR^{x}, —OR^{x}C(O)NR^{y}R^{z}, —OR^{x}C(O)OR^{y}—, —OC(O)R^{x}, —OC(O)NR^{x}R^{y}, —R^{x}NR^{y}C(O)R^{z}, —R^{x}OR^{y}, —R^{x}C(O)OR^{y}, —R^{x}C(O)NR^{y}R^{z}, —R^{x}C(O)R^{x}, —R^{x}OC(O)R^{y}, —SR^{x}, —SOR^{x}, —SO₂R^{x}, —ONO₂,
wherein R^{x}, R^{y} and R^{z} in each of the above groups can be hydrogen, substituted or unsubstituted C₁₋₈ alkyl, substituted or unsubstituted C₁₋₈ alkoxy, substituted or unsubstituted C₂₋₁₀ alkenyl, substituted or unsubstituted C₂₋₁₂ alkynyl, substituted or unsubstituted C₆₋₂₀ aryl, substituted or unsubstituted C₆₋₂₀ aryl C₁₋₈ alkyl, substituted or unsubstituted C₃₋₂₀ cycloalkyl, substituted or unsubstituted C₃₋₂₀ cycloalkylC₁₋₈ alkyl, substituted or unsubstituted C₃₋₈ cycloalkenyl, substituted or unsubstituted 5 to 14 membered heteroaryl with one or more heteroatoms selected from N, O and S, substituted or unsubstituted heteroaryl with one or more heteroatoms selected from N, O and S atomic 5 to 14 membered heteroaryl C₁₋₈ alkyl, substituted or unsubstituted 3 to 15 membered heterocycle having at least one heteroatom selected from N, O and S, substituted or unsubstituted 3 to 15 membered heterocyclyl C₁₋₈ alkyl ring having at least one heteroatom selected from N, O and S or substituted or unsubstituted amino, or any two of R^{x}, R^{y} and R^{z} may be joined to form a substituted or unsubstituted saturated or unsaturated 3-10 membered ring, which may optionally include heteroatoms which may be the same or different and are selected from O, NR^{x} or S, wherein R^{x} is hydrogen or substituted or unsubstituted alkyl, each occurrence of R⁵ is hydrogen, C₁₋₆ alkyl or halogen,
n is 0, 1, 2, 3 or 4; and
P is 0, 1, 2, 3, 4 or 5.

For example, wherein R is hydrogen, halogen, substituted or unsubstituted C₁₋₆ alkyl or OR^{a}.

In certain embodiment, wherein R^{a} is alkyl.

For example, wherein Cy¹ is selected from:

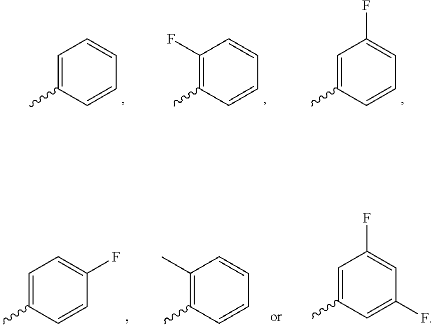

For example, wherein R¹ and R² each independently represent hydrogen or substituted or unsubstituted C₁₋₆ alkyl.

For example, wherein R³ is iodo, cyano, substituted unsubstituted alkyl, substituted or unsubstituted alkynyl, substituted or unsubstituted aryl, substituted or unsubstituted heteroaryl.

For example, wherein X is CR³ and each occurrence of R³ is independently hydrogen, halogen, hydroxyl or NH₂, for example, wherein R³ is hydrogen.

For example, wherein said PI3K inhibitors comprise:
2-[(6-Amino-9H-purin-9-yl) methyl]-6-bromo-3-phenyl-4H-chromen-4-one;
6-Bromo-2-(morpholinomethyl)-3-phenyl-4H-chromen-4-one;
6-Bromo-2-(morpholinomethyl)-3-phenyl-4H-chromen-4-one hydrochloride;
2-[(6-Amino-9H-purin-9-yl) methyl]-3-phenyl-4H-chromen-4-one;
2-(Morpholinomethyl)-3-phenyl-4H-chromen-4-one;
2-(Morpholinomethyl)-3-phenyl-4H-chromen-4-one hydrochloride;
2-[(1H-Benzo[d]imidazol-1-yl) methyl]-6-bromo-3-phenyl-4H-chromen-4-one;
6-Bromo-2-[(4-methyl-1H-benzo[d]imidazol-1-yl) methyl]-3-phenyl-4H-chromen-4-one;
2[(1 H-benzo [d] imidazol-1-yl) methyl]-3-phenyl-4H-chromen-4-one;
2-[(4-methyl-1 H-benzo [d] imidazol-1-yl)methyl]-3-phenyl-4H-chromen-4-one;
2-[(6-Chloro-9H-purin-9-yl)methyl]-3-phenyl-4H-chromen-4-one;
6-Bromo-2-[(6-chloro-9H-purin-9-yl)methyl]-3-phenyl-4H-chromen-4-one;
2-((9H-Purin-6-ylthio)methyl)-3-phenyl-4H-chromen-4-one;
2-[(1 H-Imidazol-1-yl)methyl]-3-phenyl-4H-chromen-4-one;
2-[(9H-Purin-6-ylthio)methyl]-6-bromo-3-phenyl-4H-chromen-4-one;
2-((4-Amino-1H-pyrazolo[3,4-d]pyrimidin-1-yl)methyl)-6-bromo-3-phenyl-4H-chromen-4-one;
2-[(6-Amino-9H-purin-9-yl)methyl]-6-bromo-3-(4-fluorophenyl)-4H-chromen-4-one;
2-[(6-Arnino-9H-purin-9-yl)methyl]-3-(4-fluorophenyl)-4H-chromen-4-one; 6-Bromo-3-(4-fluorophenyl)-2-(morpholinomethyl)-4H-chromen-4-one; 6-Bromo-3-(4-fluorophenyl)-2-(morpholinomethyl)-4H-chromen-4-one hydrochloride;
3-(4-fluorophenyl)-2-(morpholinomethyl)-4H-chromen-4-one; 3-(4-fluorophenyl)-2-(morpholinomethyl)-4H-chromen-4-one hydrochloride;
2-[(6-Amino-9H-purin-9-yl)methyl]-6-bromo-3-o-tolyl-4H-chromen-4-one;
7-[(6-Bromo-4-oxo-3-phenyl-4H-chromen-2-yl)methyl]-1,3-dimethyl-1H-purine-2,6(3H,7H)-dione;
2-(1-(6-Amino-9H-purin-9-yl)ethyl)-6-bromo-3-phenyl-4H-chromen-4-one;
2-(1-(9H-Purin-6-ylthio)ethyl)-6-bromo-3-phenyl-4H-chromen-4-one;
2-(1-(6-Amino-9H-purin-9-yl)ethyl)-3-phenyl-4H-chromen-4-one;
(S)-2-(1-(9H-purin-6-ylamino)ethyl)-6-bromo-3-phenyl-4H-chromen-4-one;
2-((9H-purin-6-ylamino)methyl)-6-bromo-3-phenyl-4H-chromen-4-one;
2-(1-(4-amino-1 H-pyrazolo [3,4-d]pyrimidin-1-yl)ethyl)-6-bromo-3-phenyl-4H-chromen-4-one;
2-((6-Amino-9H-purin-9-yl)methyl)-6-methoxy-3-phenyl-4H-chromen-4-one;
2-(1-(6-Amino-9H-purin-9-yl)ethyl)-6-bromo-3-(2-fluorophenyl)-4H-chromen-4-one;
2-((6-Amino-9H-purin-9-yl)methyl)-6-bromo-3-(2-fluorophenyl)-4H-chromen-4-one;
2-(1-(4-Amino-1 H-pyrazolo [3,4-d]pyrimidin-1-yl)ethyl)-3-phenyl-4H-chromen-4-one;
2-(1-(6-Amino-9H-purin-9-yl) propyl)-3-phenyl-4H-chromen-4-one; 2-(1-(6-Amino-9H-purin-9-yl)ethyl)-3-(3-fluorophenyl)-4H-chromen-4-one;
2-((6-Amino-9H-purin-9-yl)methyl)-3-(2-fluorophenyl)-4H-chromen-4-one;
2-(1-(6-Amino-9H-purin-9-yl)ethyl)-3-(2-fluorophenyl)-4H-chromen-4-one;
2-(1-(6-Amino-9H-purin-9-yl)propyl)-3-(2-fluorophenyl)-4H-chromen-4-one;
2-(1-(6-amino-9H-purin-9-yl)propyl)-3-(3-fluorophenyl)-4H-chromen-4-one;
2-(1-(6-Amino-9H-purin-9-yl)propyl)-3-(4-fluorophenyl)-4H-chromen-4-one;
2-(1-(6-amino-9H-purin-9-yl)propyl)-6-fluoro-3-phenyl-4H-chromen-4-one;
2-(1-(6-Amino-9H-purin-9-yl)ethyl)-3-(4-fluorophenyl)-4H-chromen-4-one;
2-(1-(6-Amino-9H-purin-9-yl)ethyl)-6-fluoro-3-phenyl-4H-chromen-4-one;
2-(1-(4-Amino-3-(3-methoxyphenyl)-1H-pyrazolo[3,4-d]pyrimidin-1-yl)ethyl)-3-phenyl-4H-chromen-4-one;
2-(1-(4-Amino-3-(3-hydroxyphenyl)-1 H-pyrazolo [3,4-d]pyrimidin-1-yl)ethyl)-3-phenyl-4H-chromen-4-one;
2-((9H-purin-6-ylamino)methyl)-3-phenyl-4H-chromen-4-one;
2-(1-(6-Amino-9H-purin-9-yl)ethyl)-3-o-tolyl-4H-chromen-4-one;
2-((9H-purin-6-ylamino)methyl)-3-(2-fluorophenyl)-4H-chromen-4-one;
2-((9H-purin-6-ylamino)methyl)-3-(3-fluorophenyl)-4H-chromen-4-one;
(S)-2-(1-(9H-purin-6-ylamino)ethyl)-3-(3-fluorophenyl)-4H-chromen-4-one;
2-(1-(6-amino-9H-purin-9-yl)ethyl)-6-fluoro-3-(2-fluorophenyl)-4H-chromen-4-one;
2-(1-(6-Amino-9H-purin-9-yl)ethyl)-3-(3,5-difluorophenyl)-4H-chromen-4-one;
2-(1-(6-amino-9H-purin-9-yl)ethyl)-6-fluoro-3-(3-fluorophenyl)-4H-chromen-4-one;
2-(1-(4-amino-3-(3-methoxyphenyl)-1H-pyrazolo[3,4-d]pyrimidin-1-yl)ethyl)-3-(3-fluorophenyl)-4H-chromen-4-one;
2-(1-(4-amino-3-(3-hydroxyphenyl)-1 H-pyrazolo [3,4-d] pyrimidin-1-yl) ethyl)-3-(3-fluorophenyl)-4H-chromen-4-one;
2-((4-Amino-3-(3-methoxyphenyl)-1H-pyrazolo [3,4-d] pyrimidin-1-yl) methyl)-3-phenyl-4H-chromen-4-one;
2-((4-Amino-3-(3-hydroxyphenyl)-1H-pyrazolo [3,4-d] pyrimidin-1-yl) methyl)-3-phenyl-4H-chromen-4-one;
2-((4-amino-3-(3-methoxyphenyl)-1H-pyrazolo [3,4-d] pyrimidin-1-yl) methyl)-3-(3-fluorophenyl)-4H-chromen-4-one;
2-((4-amino-3-(3-hydroxyphenyl)-1H-pyrazolo [3,4-d] pyrimidin-1-yl) methyl)-3-(3-fluorophenyl)-4H-chromen-4-one;
(R)-2-(1-(9H-purin-6-ylamino) ethyl)-3-(3-fluorophenyl)-4H-chromen-4-one;
(S)-2-(1-(9H-purin-6-ylamino) ethyl)-6-fluoro-3-phenyl-4H-chromen-4-one;
2-((4-Amino-3-iodo-1H-pyrazolo [3,4-d] pyrimidin-1-yl) methyl)-3-phenyl-4H-chromen-4-one;
2-(1-(4-amino-3-iodo-1H-pyrazolo [3,4-d] pyrimidin-1-yl) ethyl)-3-phenyl-4H-chromen-4-one;
2-(1-(4-amino-3-iodo-1H-pyrazolo [3,4-d] pyrimidin-1-yl) ethyl)-3-(3-fluorophenyl)-4H-chromen-4-one;
2-((4-amino-3-iodo-1H-pyrazolo [3,4-d] pyrimidin-1-yl) methyl)-6-fluoro-3-phenyl-4H-chromen-4-one;
2-(1-(4-amino-3-iodo-1H-pyrazolo [3,4-d] pyrimidin-1-yl) ethyl)-6-fluoro-3-phenyl-4H-chromen-4-one;
2-(1-(4-amino-3-iodo-1H-pyrazolo [3,4-d] pyrimidin-1-yl) ethyl)-6-fluoro-3-(3-fluorophenyl)-4H-chromen-4-one;
2-(1-(4-ammo-3-iodo-1H-pyrazolo [3,4-d] pyrimidin-1-yl) propyl)-3-(3-fluorophenyl)-4H-chromen-4-one;
2-((4-amino-3-(pyridin-3-yl)-1H-pyrazolo [3,4-d] pyrimidin-1-yl) methyl)-3-phenyl-4H-chromen-4-one;
2-((4-amino-3-(3-hydroxyprop-1-ynyl)-1H-pyrazolo [3,4-d] pyrimidin-1-yl) methyl)-3-phenyl-4H-chromen-4-one;
2-((4-amino-3-(1H-pyrazol-4-yl)-1H-pyrazolo [3,4-d] pyrimidin-1-yl) methyl)-3-phenyl-4H-chromen-4-one;
2-((4-amino-3-(3-(hydroxymethyl) phenyl)-1H-pyrazolo [3,4-d] pyrimidin-1-yl) methyl)-3-phenyl-4H-chromen-4-one;
2-((4-amino-3-(1H-indazol-4-yl)-1H-pyrazolo [3,4-d] pyrimidin-1-yl) methyl)-3-phenyl-4H-chromen-4-one;
2-((4-amino-3-(3-fluorophenyl)-1H-pyrazolo [3,4-d] pyrimidin-1-yl) methyl)-3-phenyl-4H-chromen-4-one
2-((4-amino-3-(3-hydroxypropyl)-1 H-pyrazolo [3,4-d] pyrimidin-1-yl) methyl)-3-phenyl-4H-chromen-4-one;
N-(3-(4-amino-14(4-oxo-3-phenyl-4H-chromen-2-yl) methyl)-1H-pyrazolo [3,4-d] pyrimidin-3-yl) phenyl) acetamide;
2-((4-amino-3-(3-fluoro-5-methoxyphenyl)-1 H-pyrazolo [3,4-d] pyrimidin-1-yl) methyl)-3-phenyl-4H-chromen-4-one;
2-((4-amino-3-(3-fluoro-5-hydroxyphenyl)-1H-pyrazolo [3,4-d] pyrimidin-1-yl) methyl)-3-phenyl-4H-chromen-4-one;
2-((4-amino-3-(3-fluoro-5-methoxyphenyl)-1 H-pyrazolo [3,4-d] pyrimidin-1-yl) methyl)-3-(3-fluorophenyl)-4H-chromen-4-one;
2-((4-amino-3-(3-fluoro-5-hydroxyphenyl)-1H-pyrazolo [3,4-d] pyrimidin-1-yl) methyl)-3-(3-fluorophenyl)-4H-chromen-4-one;
2-(1-(4-amino-3-(1 H-pyrazol-4-yl)-1 H-pyrazolo [3,4-d] pyrimidin-1-yl) ethyl)-3-phenyl-4H-chromen-4-one;
2-(1-(4-amino-3-(1H-indazol-6-yl)-1H-pyrazolo [3,4-d] pyrimidin-1-yl) ethyl)-3-phenyl-4H-chromen-4-one;
2-(1-(4-amino-3-(3-hydroxy-3-methylbut-1-ynyl)-1H-pyrazolo [3,4-d] pyrimidin-1-yl) ethyl)-3-phenyl-4H-chromen-4-one;
2-(1-(4-amino-3-(1 H-pyrazol-4-yl)-1 H-pyrazolo [3,4-d] pyrimidin-1-yl) ethyl)-3-(3-fluorophenyl)-4H-chromen-4-one;
(S)-2-(1-(9H-purin-6-ylamino) ethyl)-3-phenyl-4H-chromen-4-one;
(S)-2-(1-(9H-purin-6-ylamino) ethyl)-6-fluoro-3-(3-fluorophenyl)-4H-chromen-4-one;
2-((4-amino-3-(1H-indazol-6-yl)-1H-pyrazolo [3,4-d] pyrimidin-1-yl) methyl)-3-phenyl-4H-chromen-4-one;
2-(1-(4-amino-3-(3-fluoro-5-methoxyphenyl)-1H-pyrazolo [3,4-d] pyrimidin-1-yl) ethyl)-3-(3-fluorophenyl)-4H-chromen-4-one;
2-(1-(4-amino-3-(3-fluoro-5-hydroxyphenyl)-1H-pyrazolo [3,4-d] pyrimidin-1-yl) ethyl)-3-(3-fluorophenyl)-4H-chromen-4-one;
2-(1-(4-amino-3-(1H-indazol-4-yl)-1H-pyrazolo [3,4-d] pyrimidin-1-yl) ethyl)-3-(3-fluorophenyl)-4H-chromen-4-one;
2-(1-(4-amino-3-(3,5-dimethyl-1H-pyrazol-4-yl)-1H-pyrazolo [3,4-d] pyrimidin-1-yl) ethyl)-3-(3-fluorophenyl)-4H-chromen-4-one;
2-(1-(4-amino-3-(3-methyl-1H-indazol-6-yl)-1H-pyrazolo [3,4-d] pyrimidin-1-yl) ethyl)-3-(3-fluorophenyl)-4H-chromen-4-one;
2-(1-(4-amino-3-(1H-indazol-6-yl)-1H-pyrazolo [3,4-d] pyrimidin-1-yl) ethyl)-3-(3-fluorophenyl)-4H-chromen-4-one;
2-(1-(4-amino-3-(2-(hydroxymethyl) phenyl)-1H-pyrazolo [3,4-d] pyrimidin-1-yl) ethyl)-3-(3-fluorophenyl)-4H-chromen-4-one;
2-(1-(4-amino-3-(4-fluoro-3-methoxyphenyl)-1H-pyrazolo [3,4-d] pyrimidin-1-yl) ethyl)-3-(3-fluorophenyl)-4H-chromen-4-one;
2-(1-(4-amino-3-(4-fluoro-3-hydroxyphenyl)-1H-pyrazolo [3,4-d] pyrimidin-1-yl) ethyl)-3-(3-fluorophenyl)-4H-chromen-4-one;
2-(1-(4-amino-3-(3-hydroxyprop-1-ynyl)-1H-pyrazolo [3,4-d] pyrimidin-1-yl) ethyl)-3-(3-fluorophenyl)-4H-chromen-4-one;

2-(1-(4-amino-3-(3-fluoro-4-methoxyphenyl)-1H-pyrazolo [3,4-d] pyrimidin-1-yl) ethyl)-3-(3-fluorophenyl)-4H-chromen-4-one;
2-(1-(4-amino-3-(3-fluoro-4-hydroxyphenyl)-1H-pyrazolo [3,4-d] pyrimidin-1-yl) ethyl)-3-(3-fluorophenyl)-4H-chromen-4-one;
2-(1-(4-amino-3-(3-chloro-5-methoxyphenyl)-1H-pyrazolo [3,4-d] pyrimidin-1-yl) ethyl)-3-(3-fluorophenyl)-4H-chromen-4-one;
2-(1-(4-amino-3-(3-chloro-5-hydroxyphenyl)-1H-pyrazolo [3,4-d] pyrimidin-1-yl) ethyl)-3-(3-fluorophenyl)-4H-chromen-4-one;
2-(1-(4-amino-3-(3-(trifluoromethoxy) phenyl)-1H-pyrazolo [3,4-d] pyrimidin-1-yl) ethyl)-
3-(3-fluorophenyl)-4H-chromen-4-one;
2-(1-(4-amino-3-(4-methoxyphenyl)-1H-pyrazolo [3,4-d] pyrimidin-1-yl) ethyl)-3-(3-fluorophenyl)-4H-chromen-4-one;
2-(1-(4-amino-3-(4-hydroxyphenyl)-1H-pyrazolo [3,4-d] pyrimidin-1-yl) ethyl)-3-(3-fluorophenyl)-4H-chromen-4-one;
2-((6-amino-9H-purin-9-yl) methyl)-3-(3-fluorophenyl)-4H-chromen-4-one;
2-(1-(4-amino-3-(4-fluoro-2-methoxyphenyl)-1H-pyrazolo [3,4-d] pyrimidin-1-yl) ethyl)-3-(3-fluorophenyl)-4H-chromen-4-one;
2-(1-(4-amino-3-(4-fluoro-2-hydroxyphenyl)-1H-pyrazolo [3,4-d] pyrimidin-1-yl) ethyl)-3-(3-fluorophenyl)-4H-chromen-4-one;
2-((4-amino-3-(3-aminophenyl)-1H-pyrazolo [3,4-d] pyrimidin-1-yl) methyl)-3-phenyl-4H-chromen-4-one;
2-((4-amino-3-(3-methyl-1H-indazol-6-yl)-1H-pyrazolo [3,4-d] pyrimidin-1-yl) methyl)-3-phenyl-4H-chromen-4-one;
2-(1-(4-amino-3-(2-aminopyrimidin-5-yl)-1H-pyrazolo [3,4-d] pyrimidin-1-yl) ethyl)-3-(3-fluorophenyl)-4H-chromen-4-one;
2-(1-(4-amino-3-(1H-indol-6-yl)-1H-pyrazolo [3,4-d] pyrimidin-1-yl) ethyl)-3-(3-fluorophenyl)-4H-chromen-4-one;
2-(1-(4-amino-3-(4-chloro-3-methoxyphenyl)-1H-pyrazolo [3,4-d] pyrimidin-1-yl) ethyl)-3-(3-fluorophenyl)-4H-chromen-4-one;
2-(1-(4-amino-3-(4-chloro-3-hydroxyphenyl)-1H-pyrazolo [3,4-d] pyrimidin-1-yl) ethyl)-3-(3-fluorophenyl)-4H-chromen-4-one;
2-(1-(4-amino-3-(2-chloro-5-methoxyp enyl)-1H-pyrazolo [3,4-d] pyrimidin-1-yl) ethyl)-3-(3-fluorophenyl)-4H-chromen-4-one;
2-(1-(4-amino-3-(2-chloro-5-hydroxyphenyl)-1H-pyrazolo [3,4-d] pyrimidin-1-yl) ethyl)-3-(3-fluorophenyl)-4H-chromen-4-one;
2-(1-(4-amino-3-(3,4-dimethoxyphenyl)-1H-pyrazolo [3,4-d] pyrimidin-1-yl) ethyl)-3-(3-fluorophenyl)-4H-chromen-4-one;
2-(1-(4-amino-3-(3,4-dihydroxyphenyl)-1 H-pyrazolo [3,4-d] pyrimidin-1-yl) ethyl)-3-(3-fluorophenyl)-4H-chromen-4-one;
2-((4-amino-3-(3-methyl-1 H-indazol-6-yl)-1 H-pyrazolo [3,4-d] pyrimidin-1-yl) methyl)-3-(3-fluorophenyl)-4H-chromen-4-one;
2-(1-(4-amino-3-(1H-indol-5-yl)-1H-pyrazolo [3,4-d] pyrimidin-1-yl) ethyl)-3-(3-fluorophenyl)-4H-chromen-4-one;
2-(1-(4-Amino-3-(3-methyl-1H-indol-5-yl)-1H-pyrazolo [3,4-d] pyrimidin-1-yl) ethyl)-3-(3-fluorophenyl)-4H-chromen-4-one;
tert-butyl-(5-(4-amino-1-(1-(3-(3-fluorophenyl)-4-oxo-4H-chromen-2-yl) ethyl)-1 H-pyrazolo [3,4-d] pyrimidin-3-yl) thiophen-2-yl) methylcarbamate
2-(1-(4-amino-3-(5-(aminomethyl) thiophen-2-yl)-1H-pyrazolo [3,4-d] pyrimidin-1-yl) ethyl)-3-(3-fluorophenyl)-4H-chromen-4-one;
2-((4-amino-3-(3-methyl-1 H-indazol-6-yl)-1 H-pyrazolo [3,4-d] pyrimidin-1-yl) methyl)-6-fluoro-3-phenyl-4H-chromen-4-one;
2-(1-(4-amino-3-(3-methyl-1 H-indazol-6-yl)-1 H-pyrazolo [3,4-d] pyrimidin-1-yl) ethyl)-3-phenyl-4H-chromen-4-one;
2-(1-(4-amino-3-(3-methyl-1 H-indazol-6-yl)-1 H-pyrazolo [3,4-d] pyrimidin-1-yl) ethyl)-6-fluoro-3-phenyl-4H-chromen-4-one;
2-(1-(4-amino-3-(3-methyl-1H-indazol-5-yl)-1 H-pyrazolo [3,4-d] pyrimidin-1-yl) ethyl)-3-(3-fluorophenyl)-4H-chromen-4-one;
N-(4-(4-amino-1-(1--(3-(3-fluorophenyl)-4-oxo-4H-chromen-2-yl) ethyl)-1 H-pyrazolo [3,4-d] pyrimidin-3-yl) phenyl) acetamide;
2-(1-(4-amino-3-(4-aminophenyl)-1 H-pyrazolo [3,4-d] pyrimidin-1-yl) ethyl)-3-(3-fluorophenyl)-4H-chromen-4-one;
2-(1-(4-amino-3-(3-methyl-1H-indazol-6-yl)-1H-pyrazolo [3,4-d] pyrimidin-1-yl) ethyl)-6-fluoro-3-(3-fluorophenyl)-4H-chromen-4-one;
2-(1-(4-amino-3-(2,3-dihydrobenzofuran-5-yl)-1 H-pyrazolo [3,4-d] pyrimidin-1-yl) ethyl)-3-(3-fluorophenyl)-4H-chromen-4-one;
2-(1-(4-amino-3-(3-ethyl-1H-indazol-6-yl)-1H-pyrazolo [3,4-d] pyrimidin-1-yl) ethyl)-3-(3-fluorophenyl)-4H-chromen-4-one;
2-(1-(4-amino-3-(3-methyl-1 H-indol-6-yl)-1 H-pyrazolo [3,4-d] pyrimidin-1-yl) ethyl)-3-(3-fluorophenyl)-4H-chromen-4-one;
2-(1-(4-amino-3-(2-methoxypyrimidin-5-yl)-1 H-pyrazolo [3,4-d] pyrimidin-1-yl) ethyl)-3-(3-fluorophenyl)-4H-chromen-4-one;
4-(4-amino-1-(1-(3-(3-fluorophenyl)-4-oxo-4H-chromen-2-yl) ethyl)-1 H-pyrazolo [3,4-d] pyrimidin-3-yl) thiophene-2-carbaldehyde;
2-(1-(4-amino-3-(5-(hydroxymethyl) thiophen-3-yl)-1H-pyrazolo [3,4-d] pyrimidin-1-yl) ethyl)-3-(3-fluorophenyl)-4H-chromen-4-one;
2-(1-(4-amino-3-(2-methyl-1H-benzo[d]imidazol-5-yl)-1H-pyrazolo [3,4-d] pyrimidin-1-yl) ethyl)-3-(3-fluorophenyl)-4H-chromen-4-one;
2-(1-(4-amino-3-(3-methyl-1 H-indazol-6-yl)-1 H-pyrazolo [3,4-d] pyrimidin-1-yl) propyl)-3-(3-fluorophenyl)-4H-chromen-4-one;
2-(1-(4-amino-3-(3-methyl-1H-indol-6-yl)-1H-pyrazolo [3,4-d] pyrimidin-1-yl) ethyl)-3-(3-fluorophenyl)-4H-chromen-4-one;
2-((6-amino-9H-purin-9-yl) methyl)-6-fluoro-3-phenyl-4H-chromen-4-one;
2-((6-amino-9H-purin-9-yl) methyl)-6-fluoro-3-(3-fluorophenyl)-4H-chromen-4-one;
2-((4-amino-3-(3-fluoro-5-methoxyphenyl)-1H-pyrazolo [3,4-d] pyrimidin-1-yl) methyl)-6-fluoro-3-(3-fluorophenyl)-4H-chromen-4-one;
2-((4-amino-3-(3-fluoro-5-hydroxyphenyl)-1H-pyrazolo [3,4-d] pyrimidin-1-yl) methyl)-6-fluoro-3-(3-fluorophenyl)-4H-chromen-4-one;
2-(1-(4-amino-3-(3-methoxyphenyl)-1H-pyrazolo [3,4-d] pyrimidin-1-yl) ethyl)-6-fluoro-3-(3-fluorophenyl)-4H-chromen-4-one;
2-(1-(4-amino-3-(3-hydroxyphenyl)-1H-pyrazolo [3,4-d] pyrimidin-1-yl) ethyl)-6-fluoro-3-(3-fluorophenyl)-4H-chromen-4-one;
2-((9H-purin-6-ylamino) methyl)-6-fluoro-3-(3-fluorophenyl)-4H-chromen-4-one;
2-((9H-purin-6-ylamino) methyl)-6-fluoro-3-phenyl-4H-chromen-4-one;
(R)-2-(1-(9H-purin-6-ylamino) ethyl)-6-fluoro-3-(3-fluorophenyl)-4H-chromen-4-one;
2-((4-amino-3-(1H-pyrazol-4-yl)-1H-pyrazolo [3,4-d] pyrimidin-1-yl) methyl)-6-fluoro-3-phenyl-4H-chromen-4-one;
2-(1-(4-amino-3-(3,5-difluoro-4-methoxyphenyl)-1H-pyrazolo [3,4-d] pyrimidin-1-yl) ethyl)-3-(3-fluorophenyl)-4H-chromen-4-one;
2-(1-(4-amino-3-(3,5-difluoro-4-hydroxyphenyl)-1H-pyrazolo [3,4-d] pyrimidin-1-yl) ethyl)-3-(3-fluorophenyl)-4H-chromen-4-one;
2-((4-amino-3-(3,5-difluoro-4-methoxyphenyl)-1H-pyrazolo [3,4-d] pyrimidin-1-yl) methyl)-6-fluoro-3-(3-fluorophenyl)-4H-chromen-4-one;
2-((4-amino-3-(3,5-difluoro-4-hydroxyphenyl)-1H-pyrazolo [3,4-d] pyrimidin-1-yl) methyl)-6-fluoro-3-(3-fluorophenyl)-4H-chromen-4-one;
(+)-2-(1-(4-amino-3-(3-methyl-1H-indazol-6-yl)-1H-pyrazolo [3,4-d] pyrimidin-1-yl) ethyl)-3-(3-fluorophenyl)-4H-chromen-4-one;
(−)-2-(1-(4-amino-3-(3-methyl-1H-indazol-6-yl)-1H-pyrazolo [3,4-d] pyrimidin-1-yl) ethyl)-3-(3-fluorophenyl)-4H-chromen-4-one;
2-(1-(4-amino-3-(3,5-dimethoxyphenyl)-1H-pyrazolo [3,4-d] pyrimidin-1-yl) ethyl)-3-(3-fluorophenyl)-4H-chromen-4-one;
2-(1-(4-amino-3-(4-methoxy-3,5-dimethylphenyl)-1H-pyrazolo [3,4-d] pyrimidin-1-yl) ethyl)-3-(3-fluorophenyl)-4H-chromen-4-one;
2-(1-(4-amino-3-(2-fluoro-5-isopropoxyphenyl)-1H-pyrazolo [3,4-d] pyrimidin-1-yl) ethyl)-
3-(3-fluorophenyl)-4H-chromen-4-one;
2-(1-(4-amino-3-(2,3-dihydrobenzo[b] [1,4] dioxin-6-yl)-1H-pyrazolo [3,4-d] pyrimidin-1-yl) ethyl)-3-(3-fluorophenyl)-4H-chromen-4-one;
2-(1-(4-amino-3-(1-benzyl-1H-pyrazol-4-yl)-1H-pyrazolo [3,4-d] pyrimidin-1-yl) ethyl)-3-(3-fluorophenyl)-4H-chromen-4-one;
2-(1-(4-amino-3-(2-methylpyridin-4-yl)-1H-pyrazolo [3,4-d] pyrimidin-1-yl) ethyl)-3-(3-fluorophenyl)-4H-chromen-4-one;
2-(1-(4-amino-3-(3,4-dihydro-2H-benzo[b] [1,4] dioxepin-7-yl)-1H-pyrazolo [3,4-d] pyrimidin-1-yl) ethyl)-3-(3-fluorophenyl)-4H-chromen-4-one;
2-(1-(4-amino-3-(6-morpholinopyridin-3-yl)-1H-pyrazolo [3,4-d] pyrimidin-1-yl) ethyl)-3-(3-fluorophenyl)-4H-chromen-4-one;
2-(1-(4-amino-3-(dibenzo [b, d] furan-4-yl)-1H-pyrazolo [3,4-d] pyrimidin-1-yl) ethyl)-3-(3-fluorophenyl)-4H-chromen-4-one;
2-(1-(4-amino-3-(4-phenoxyphenyl)-1H-pyrazolo [3,4-d] pyrimidin-1-yl) ethyl)-3-(3-fluorophenyl)-4H-chromen-4-one;
2-(1-(4-amino-3-(4-(benzyloxy)-3-chlorophenyl)-1H-pyrazolo [3,4-d] pyrimidin-1-yl) ethyl)-3-(3-fluorophenyl)-4H-chromen-4-one;
2-(1-(4-amino-3-(3-chloro-4-isopropoxyphenyl)-1H-pyrazolo [3,4-d] pyrimidin-1-yl) ethyl)-
3-(3-fluorophenyl)-4H-chromen-4-one;
2-(1-(4-amino-3-(3-(dimethylamino) phenyl)-1H-pyrazolo [3,4-d] pyrimidin-1-yl) ethyl)-3-(3-fluorophenyl)-4H-chromen-4-one;
2-(1-(4-amino-3-(4-ethoxy-3-fluorophenyl)-1H-pyrazolo [3,4-d] pyrimidin-1-yl) ethyl)-3-(3-fluorophenyl)-4H-chromen-4-one;
2-(1-(4-amino-3-(4-isopropoxyphenyl)-1H-pyrazolo [3,4-d] pyrimidin-1-yl) ethyl)-3-(3-fluorophenyl)-4H-chromen-4-one;
2-(1-(4-amino-3-(4-(trifluoromethoxy) phenyl)-1H-pyrazolo [3,4-d] pyrimidin-1-yl) ethyl)-3-(3-fluorophenyl)-4H-chromen-4-one;
2-(1-(3-(4-acetylphenyl)-4-amino-1H-pyrazolo [3,4-d] pyrimidin-1-yl) ethyl)-3-(3-fluorophenyl)-4H-chromen-4-one;
2-(1-(4-amino-3-(4-(benzyloxy) phenyl)-1H-pyrazolo [3,4-d] pyrimidin-1-yl) ethyl)-3-(3-fluorophenyl)-4H-chromen-4-one;
2-(1-(4-amino-3-(4-(dimethylamino) phenyl)-1H-pyrazolo [3,4-d] pyrimidin-1-yl) ethyl)-3-(3-fluorophenyl)-4H-chromen-4-one;
2-(1-(4-amino-3-(4-(methylsulfonyl) phenyl)-1H-pyrazolo [3,4-d] pyrimidin-1-yl) ethyl)-3-(3-fluorophenyl)-4H-chromen-4-one;
2-(1-(4-amino-3-(3-ethoxyphenyl)-1 H-pyrazolo [3,4-d] pyrimidin-1-yl) ethyl)-3-(3-fluorophenyl)-4H-chromen-4-one;
2-(1-(4-amino-3-(benzo[b]thiophen-2-yl)-1H-pyrazolo [3,4-d] pyrimidin-1-yl) ethyl)-3-(3-fluorophenyl)-4H-chromen-4-one;
2-(1-(4-amino-3-(5-chlorothiophen-2-yl)-1H-pyrazolo [3,4-d] pyrimidin-1-yl) ethyl)-3-(3-fluorophenyl)-4H-chromen-4-one;
2-(1-(4-amino-3-(3,5-dimethylisoxazol-4-yl)-1H-pyrazolo [3,4-d] pyrimidin-1-yl) ethyl)-3-(3-fluorophenyl)-4H-chromen-4-one;
2-(1-(4-amino-3-(3-propoxyphenyl)-1H-pyrazolo [3,4-d] pyrimidin-1-yl) ethyl)-3-(3-fluorophenyI)-4H-chromen-4-one;
2˜(1-(4-amino-3-(furan-2-yl)-1H-pyrazolo [3,4-d] pyrimidin-1-yl) ethyl)-3-(3-fluorophenyl)-4H-chromen-4-one;
2-(1-(4-amino-3-(4-ethoxyphenyl)-1H-pyrazolo [3,4-d] pyrimidin-1-yl) ethyl)-3-(3-fluorophenyl)-4H-chromen-4-one;
2-(1-(4-amino-3-(3-chloro-4-methoxyphenyl)-1H-pyrazolo [3,4-d] pyrimidin-1-yl) ethyl)-3-(3-fluorophenyl)-4H-chromen-4-one;
2-(1-(4-amino-3-(3-fluoro-4-isopropoxyphenyl)-1H-pyrazolo [3,4-d] pyrimidin-1-yl) ethyl)-
3-(3-fluorophenyl)-4H-chromen-4-one;
2-(1-(4-amino-3-(6-fluoropyridin-3-yl)-1H-pyrazolo [3,4-d] pyrimidin-1-yl) ethyl)-3-(3-fluorophenyl)-4H-chromen-4-one;
2-(1-(4-amino-3-(pyrimidin-5-yl)-1H-pyrazolo [3,4-d] pyrimidin-1-yl) ethyl)-3-(3-fluorophenyl)-4H-chromen-4-one;
2-(1-(4-amino-3-(3-(methoxymethyl) phenyl)-1H-pyrazolo [3,4-d] pyrimidin-1-yl) ethyl)-3-(3-fluorophenyl)-4H-chromen-4-one;
2-(1-(4-amino-3-(6-hydroxynaphthalen-2-yl)-1H-pyrazolo [3,4-d] pyrimidin-1-yl) ethyl)-3-(3-fluorophenyl)-4H-chromen-4-one;
2-(1-(4-amino-3-(3-isopropoxyphenyl)-1H-pyrazolo [3,4-d] pyrimidin-1-yl) ethyl)-3-(3-(3-fluorophenyl)-4H-chromen-4-one;
2-(1-(4-amino-3-(1-methyl-1H-pyrazol-4-yl)-1H-pyrazolo [3,4-d] pyrimidin-1-yl) ethyl)-3-(3-fluorophenyl)-4H-chromen-4-one;
6-Fluoro-3-(3-fluorophenyl)-2-(1-(4-methoxyphenylamino) ethyl)-4H-chromen-4-one;
2-(1-(4-Chloro-7H-pyrrolo [2,3-d] pyrimidin-7-yl) emyl)-3-(3-fluorophenyl)-4H-chromen-4-one; 2-(1-(4-Chloro-1H-pyrazolo [3,4-b] pyridin-1-yl) ethyl)-3-(3-fluorophenyl)-4H-chromen-4-one;
2-(1-(4-Chloro-1H-pyrazolo [3,4-d] pyrimidin-1-yl) ethyl)-3-(3-fluorophenyl)-4H-chromen-4-one;
2-(1-(4-Chloro-5H-pyrrolo [3,2-d] pyrimidin-5-yl) ethyl)-3-(3-fluorophenyl)-4H-chromen-4-one;
2-(1-(4-amino-3-(1,3-dimethyl-1H-indazol-6-yl)-1H-pyrazolo [3,4-d] pyrimidin-1-yl) ethyl)-3-(3-fluorophenyl)-4H-chromen-4-one;
2-(1-(4-amino-3-(2,3-dimethyl-2H-indazol-6-yl)-1H-pyrazolo [3,4-d] pyrimidin-1-yl) ethyl)-3-(3-fluorophenyl)-4H-chromen-4-one;
2-(1-(4-amino-3-(6-methoxynaphthalen-2-yl)-1 H-pyrazolo [3,4-d] pyrimidin-1-yl) ethyl)-3-(3-fluorophenyl)-4H-chromen-4-one;
2-(1-(4-amino-3-(benzo[b]thiophen-3-yl)-1H-pyrazolo [3,4-d] pyrimidin-1-yl) ethyl)-3-(3-fluorophenyl)-4H-chromen-4-one;
2-(1-(4-amino-3-(2,4-dimethoxypyrimidin-5-yl)-1 H-pyrazolo [3,4-d] pyrimidin-1-yl) ethyl)-
3-(3-fluorophenyl)-4H-chromen-4-one;
2-(1-(4-amino-3-(6-ethoxynaphthalen-2-yl)-1 H-pyrazolo [3,4-d] pyrimidin-1-yl) ethyl)-3-(3-fluorophenyl)-4H-chromen-4-one;
3-(4-amino-1-(1-(3-(3-fluorophenyl)-4-oxo-4H-chromen-2-yl) ethyl)-1 H-pyrazolo [3,4-d] pyrimidin-3-yl)-N-cyclopropylbenzamide;
2-(1-(4-amino-3-(3-(morpholine-4-carbonyl) phenyl)-1 H-pyrazolo [3,4-d] pyrimidin-1-yl) ethyl)-3-(3-fluorophenyl)-4H-chromen-4-one;
2-(1-(4-amino-3-(3-(difluoromethoxy) phenyl)-1 H-pyrazolo [3,4-d] pyrimidin-1-yl) ethyl)-3-(3-fluorophenyl)-4H-chromen-4-one;
5-(4-amino-1-(1-(3-(3-fluorophenyl)-4-oxo-4H-chromen-2-yl) ethyl)-1 H-pyrazolo [3,4-d] pyrimidin-3-yl) furan-2-carbaldehyde and pharmaceutically acceptable salts thereof;
   or their enantiomers, mixtures of enantiomers, mixtures of two or more diastereoisomers, isotopic variants, pharmaceutically acceptable salts, solvates, hydrates or pro-drug.

In certain embodiment, wherein said PI3K inhibitor comprise: 2-(1-(9H-purin-6-ylamino) propyl group)-3-(3-fluorophenyl)-4H-chromen-4-one or its enantiomers, mixtures of enantiomers, mixtures of two or more diastereoisomers, isotopic variants, pharmaceutically acceptable salts, solvates, hydrates or prodrugs.

In certain embodiment, wherein said PI3K inhibitor comprise: (S)-2-(1-(9H-purin-6-ylamino) propyl group)-3-(3-fluorophenyl)-4H-chromen-4-one (Tenalisib, also named CN401) or its pharmaceutically acceptable salts, solvates, hydrates or prodrugs.

In certain embodiment, the pharmaceutical combination may include an anti-PD-L1 antibody or its antigen-binding fragment thereof and PI3K inhibitor.

For example, the pharmaceutical combination may include: (i) an anti-PD-L1 antibody or antigen-binding fragment thereof comprising atezolizumab, avelumab, BMS-936559, MPDL3280A or durvalumab or an antigen thereof binding fragment; (ii) PI3Kδ/γ dual inhibitor, said PI3Kδ/γ dual inhibitor comprise 2-(1-(9H-purin-6-ylamino) propyl group)-3-(3-fluorophenyl)-4H-chromen-4-one or its enantiomers, mixtures of enantiomers, mixtures of two or more diastereoisomers, isotopic variants, pharmaceutically acceptable salts, solvates, hydrates or prodrugs.

For another example, said pharmaceutical combination may include: (i) anti-PD-L1 antibody selected from the following group: Atezolizumab, Avelumab, BMS-936559, MPDL3280A, durvalumab or its combination; (ii) 2-(1-(9H-purin-6-ylamino) propyl group)-3-(3-fluorophenyl)-4H-chromen-4-one or its pharmaceutically acceptable salts, solvates, hydrates or prodrugs.

In certain embodiment, said pharmaceutical combination may include TGFβ inhibitor and PI3K inhibitor.

For example, the pharmaceutical combination may include: (i) TGFβ inhibitors, said TGFβ inhibitors include: anti-TGFβ antibodies or antigen-binding fragments thereof, small molecule TGFβ inhibitors or combinations thereof, said TGFβ antibodies or antigen-binding fragments thereof including Fresolimumab or an antigen-binding fragment thereof, the small molecule TGFβ inhibitors include SB525334, SD-208, SB431542, LY2109761, LY2157299 (Galunisertib), GW788388, RepSox, SIS3, LDN-193189, EW-7197, LY364947 or combinations thereof; (ii) PI3Kδ/γ dual inhibitor, said PI3Kδ/γ dual inhibitor comprises 2-(1-(9H-purin-6-ylamino) propyl group)-3-(3-fluorophenyl)-4H-chromen-4-one or its enantiomers, mixtures of enantiomers, mixtures of two or more diastereoisomers, isotopic variants, pharmaceutically acceptable salts, solvates, hydrates or prodrugs.

For another example, the pharmaceutical combination may comprise Fresolimumab or its antigen-binding fragment and (S)-2-(1-(9H-purin-6-ylamino) propyl group)-3-(3-fluorophenyl)-4H-chromen-4-one or a pharmaceutically acceptable salt, solvate, hydrate or prodrug thereof.

For another example, the pharmaceutical combination may comprise Fresolimumab or its antigen-binding fragment, SB431542 and (S)-2-(1-(9H-purin-6-ylamino) propyl group)-3-(3-fluorophenyl)-4H-chromen-4-one or a pharmaceutically acceptable salt, solvate, hydrate or prodrug thereof.

In certain embodiment, the pharmaceutical combination may comprise PD-1 inhibitor, TGF β inhibitor and PI3K inhibitor.

For example, the pharmaceutical combination may comprise (i) an anti-PD-1 antibody or an antigen-binding fragment thereof, the anti-PD-1 antibody or an antigen-binding fragment thereof including Nivolumab, Pembrolizumab, Pidilizumab antibody or AMP-514 or antigen-binding fragment thereof; (ii) PI3Kδ/γ dual inhibitors, said PI3Kδ/γ dual inhibitor comprising 2-(1-(9H-purin-6-ylamino) propyl group)-3-(3-fluorophenyl)-4H-chromen-4-one, or its enantiomer, a mixture of enantiomers, a mixture of two or more diastereoisomers, or isotopic variants; or pharmaceutically acceptable salts, solvates, hydrates or prodrugs thereof; (iii) anti-TGFβ antibodies or antigen-binding fragments thereof, small molecule TGFβ inhibitors or combinations thereof, said TGFβ antibodies or antigens-binding fragments include Fresolimumab or antigen-binding fragments thereof, and said small molecule TGFβ inhibitors include SB525334, SD-208, SB431542, LY2109761, LY2157299 (Galunisertib), GW788388, RepSox, SIS3, LDN-193189, EW-7197, LY364947 or their combination.

For another example, the pharmaceutical combination may include: (i) an anti-PD-1 antibody selected from the group consisting of nivolumab, pembrolizumab, Pidilizumab or AMP-514 or a combination thereof; (ii) (S)-2-(1-(9H-purin-6-ylamino) propyl group)-3-(3-fluorophenyl)-4H-chromen-4-one or a pharmaceutically acceptable salt thereof, a solvate, hydrate or prodrug; (iii) Fresolimumab or an antigen-binding fragment thereof and/or SB431542.

In one embodiment, the pharmaceutical combination may comprise PD-1 inhibitor, TGF β inhibitor and PI3K inhibitor.

For example, the pharmaceutical combination may comprise (i) an anti-PD-L1 antibody or antigen-binding fragment thereof comprising atezolizumab, avelumab, BMS-936559, MPDL3280A or durvalumab or an antigen-binding fragments; (ii) PI3Kδ/γ dual inhibitors comprising 2-(1-(9H-purin-6-ylamino) propyl group)-3-(3-fluorophenyl)-4H-chromen-4-one, or an enantiomer, a mixture of enantiomers, a mixture of two or more diastereoisomers, or an isotopic variant thereof; or a pharmaceutically acceptable salts, solvates, hydrates or prodrugs; (iii) anti-TGFβ antibodies or antigen-binding fragments thereof comprising Fresolimumab or antigen-binding fragments thereof.

For another example, the pharmaceutical combination may include: (i) an anti-PD-L1 antibody selected from the group consisting of Atezolizumab, Avelumab, BMS-936559, MPDL3280A or durvalumab or a combination thereof; (ii) (S)-2-(1-(9H-purin-6-ylamino) propyl group)-3-(3-fluorophenyl)-4H-chromen-4-one or its pharmaceutically acceptable salt, solvate, hydrate or prodrug (iii) Fresolimumab or an antigen-binding fragment thereof.

In one embodiment, the pharmaceutical combination may comprise the PD-L1/TGFβ dual inhibitor and a PI3K inhibitor.

In certain embodiment, the PD-L1/TGFβ dual inhibitors may be those described in PCT/CN2019124535, all of which are incorporated by reference into this application. For example, the PD-L1/TGFβ dual inhibitor may be WBP1126 (also known as CN202), or a variant or biosimilar thereof. The WBP1126 may comprise two polypeptides, the polypeptides sequentially comprise the heavy chain variable region (VH), CH2, CH3 domains of the anti-PD-L1 antibody from the N-terminus to the C-terminus, and TGFβRII or a functionally active fragment thereof. The C-terminus of the CH3 domain is connected to the N-terminus of the TGFβRII or its functionally active fragment through a linker; for example, the polypeptide may have the amino acid sequence shown in Table 1:

TABLE 1

| WBP1126-U15T1.G1- | | | |
|---|---|---|---|
| 1.uIgG1 | | SEQ ID NO | Amino acid sequence |
| VHH | HCDR1 | 1 | GHFSNLAVN |
| | HCDR2 | 2 | GILWSGGSTFYADSVKG |
| | HCDR3 | 3 | GTN |
| | VHH | 4 | EVQLVESGGGLVQPGGSLRLSCAASGHESNLA |
| | | | VNWFRQAPGKERELVAGILWSGGSTFYADSV |
| | | | KGRFTISRGNAENMLYLQMNSLRAEDTAVYY |
| | | | CNTGTNWGQGTLVTVSS |

| | | |
|---|---|---|
| linker | 5 | GGGGSGGGGSGGGGSGGGGS |
| TGFβRII extracellular | 6 | IPPHVQKSVNNDMIVTDNNGAVKFPQLCKFC |
| domain | | DVRFSTCDNQKSCMSNCSITSICEKPQEVCVA |
| | | VWRKNDENITLETVCHDPKLPYHDFILEDAAS |
| | | PKCIMKEKKKPGETFFMCSCSSDECNDNIIFSE |
| | | EYNTSNPD |
| Sequence full | 7 | EVQLVESGGGLVQPGGSLRLSCAASGHESNLA |
| length | | VNWFRQAPGKERELVAGILWSGGSTFYADSV |
| | | KGRFTISRGNAENMLYLQMNSLRAEDTAVYY |
| | | CNTGTNWGQGTLVTVSSEPKSCDKTHTCPPC |
| | | PAPELLGGPSVFLFPPKPKDTLMISRTPEVTCV |
| | | VVDVSHEDPEVKFNWYVDGVEVHNAKTKPR |
| | | EEQYNSTYRVVSVLTVLHQDWLNGKEYKCK |
| | | VSNKALPAPIEKTISKAKGQPREPQVYTLPPSR |
| | | EEMTKNQVSLTCLVKGFYPSDIAVEWESNGQP |
| | | ENNYKTTPPVLDSDGSFFLYSKLTVDKSRWQ |
| | | QGNVFSCSVMHEALHNHYTQKSLSLSPGGGG |
| | | SGGGGSGGGGSGGGGSIPPHVQKSVNNDMIV |
| | | TDNNGAVKFPQLCKFCDVRFSTCDNQKSCMS |
| | | NCSITSICEKPQEVCVAVWRKNDENITLETVC |
| | | HDPKLPYHDFILEDAASPKCIMKEKKKPGETF |
| | | FMCSCSSDECNDNIIFSEEYNTSNPD |

In certain embodiment, wherein the PD-L1/TGFβ dual inhibitor comprises a first polypeptide and a second polypeptide, wherein the first polypeptide comprises at least: (i) a heavy chain of an anti-PD-L1 antibody variable region; and (ii) TGFβRII or a functionally active fragment thereof; wherein the second polypeptide comprises at least the light chain variable region of an anti-PD-L1 antibody; wherein the heavy chain variable region of the first polypeptide and the light chain variable regions of the second polypeptide are capable of specifically binding to PD-L1.

In certain embodiment, wherein PD-L1/TGFβ dual inhibitor further comprises a linker, said linker connects the C-terminus of the heavy chain variable region of the anti-PD-L1 antibody or antigen-binding fragment thereof with the TGFβRII or the N-terminus of its functionally active fragments.

In certain embodiment, wherein the PD-L1/TGFβ dual inhibitor comprises a first polypeptide and a second polypeptide:
wherein the first polypeptide comprises the heavy chain variable region (VH) of the anti-PD-L1 antibody, the CH1 domain, and TGFβRII or its functionally active fragment, and the C-terminus of the CH1 domain is connected to the The TGFβRII or its functionally active fragment N-terminus is connected by a linker; and the second polypeptide comprises a light chain variable region (VL) and a light chain constant region (CL) of an anti-PD-L1 antibody from the N-terminus to the C-terminus.

In certain embodiment, wherein the PD-L1/TGFβ dual inhibitor comprises a first polypeptide and a second polypeptide:

The first polypeptide comprises the heavy chain variable region (VH), CH1, CH2, CH3 domains and TGFβRII or its functionally active fragments of the anti-PD-L1 antibody sequentially from the N-terminus to the C-terminus, and the CH3 domain The C-terminus is connected to the N-terminus of the TGFβRII or its functionally active fragment through a linker; and the second polypeptide comprises a light chain variable region (VL) and a light chain of an anti-PD-L1 antibody from the N-terminus to the C-terminus Constant region (CL).

In certain embodiment, wherein said linker is a peptide linker.

In certain embodiment, wherein the first polypeptide has at least about 80% sequence identity to a polypeptide having the same function as: M7824 or SHR-1701.

In certain embodiment, wherein the second polypeptide has at least about 80% sequence identity to a polypeptide having the same function as: M7824 or SHR-1701.

In certain embodiment, the PD-L1/TGFβ dual inhibitors may be those described in PCT/EP2015052781 and PCT/CN2018086451, all of which are incorporated by reference into this application.

In certain embodiment, wherein the PD-L1/TGFβ dual inhibitor comprises M7824, SHR-1702, or variant or biosimilar or their combination.

In certain embodiment, the PD-L1/TGFβ dual inhibitor and the PI3K inhibitor may not be mixed with each other in the drug combination.

In certain embodiment, the PD-L1/TGFβ dual inhibitor and the PI3K inhibitor can be independently contained in separate containers.

### Pharmaceutical Composition and Uses

In another aspect, the present application provides a pharmaceutical composition, the pharmaceutical combination comprises the aforementioned PI3K inhibitor and a second therapeutic agent, and optionally one or more pharmaceutically acceptable carriers or excipients

In certain embodiment, the second therapeutic agent is the aforementioned PD-L1/TGFβ dual inhibitor.

In certain embodiment, the PI3K inhibitor is the aforementioned PI3Kδ/γ dual inhibitor.

In some embodiment, the pharmaceutical composition comprises a PD-L1/TGFβ dual inhibitor and a PI3Kδ/γ dual inhibitor.

For example, the pharmaceutical composition may comprise:
i) WBP1126 or a variant or biosimilar thereof; and
ii) Tenalisib or a pharmaceutically acceptable salt, solvate, hydrate or prodrug thereof.

In certain embodiment, the second therapeutic agent is the aforementioned anti-PD-L1 antibody, and the PI3K inhibitor is the aforementioned PI3Kδ/γ dual inhibitor.

For example, the pharmaceutical composition may include: i) nivolumab, pembrolizumab, pidilizumab, or combinations thereof; and ii) tenalisib or a pharmaceutically acceptable salt, solvate, or hydrate thereof or prodrug.

In certain embodiment, the second therapeutic agent is the aforementioned PD-L1/TGFβ dual inhibitor, and the PI3K inhibitor is a PI3Kα inhibitor.

For example, the pharmaceutical composition may comprise:
i) WBP1126 or a variant or biosimilar thereof, and
ii) Alplisib or its pharmaceutically acceptable salt, solvate, hydrate or prodrug.

In certain embodiment, wherein said second therapeutic agent and said PI3K inhibitor are present in a single or separate dosage form.

In certain embodiment, wherein the pharmaceutical composition includes, each unit dosage form (unit dosage form) contains 1-1000 mg dose of the first therapeutic agent, such as PI3K inhibitor active substance (such as CN401 (Tenalisib)), For example 1, 2, 3, 4, 5, 6, 7, 8, 9, 10, 11, 12, 13, 14, 15, 16, 17, 18, 19, 20, 25, 30, 40, 50, 60 , 70, 80, 90, 100, 110, 120, 130, 140, 150, 160, 170, 180, 190, 200, 250, 300, 350, 400, 450, 500, 600, 700, 800, 900, 1000 mg or a value between any two of the above values; and, each unit dosage form (unit dosage form) contains a second therapeutic agent at a dose of 1-1000 mg, such as a PD-L1 inhibitor, a TGFβ inhibitor, a PD-L1 inhibitor, PD-L1/TGFβ dual inhibitors (such as CN202 (WBP1126)) or PD-1/TGFβ dual inhibitors or a combination thereof, such as 1, 2, 3, 4, 5, 6, 7, 8, 9, 10, 11, 12, 13, 14, 15, 16, 17, 18, 19, 20, 25, 30, 40, 50, 60, 70, 80, 90, 100, 110, 120, 130, 140, 150, 160, 170, 180, 190, 200, 250, 300, 350, 400, 450, 500, 600, 700, 800, 900, 1000 mg or a value between any two of the above values.

In certain embodiment, the pharmaceutical composition can further include a third active substance.

In certain embodiment, wherein the third active substance may be selected from vincristine, vinblastine, vindesine, etoposide, docetaxel, paclitaxel (such as nab-paclitaxel), irinotecan, vinorelbine, mitoxantrone, vinflunine, topotecan or any combination thereof.

In another aspect, the present application provides a method for treating and/or preventing tumors, which comprises administering to a subject in need: an effective amount of the aforementioned pharmaceutical combination, or the aforementioned pharmaceutical composition.

In certain embodiment, the method includes administering to a subject in need: an effective amount of the aforementioned PI3K inhibitor and the aforementioned immune checkpoint inhibitor.

In certain embodiment, the method comprises administering to a subject in need: an effective amount of the aforementioned PI3K inhibitor and the aforementioned TGFβ inhibitor.

In certain embodiment, the method includes administering to a subject in need: an effective amount of the aforementioned PI3K inhibitor, the aforementioned TGFβ inhibitor, and the aforementioned immune checkpoint inhibitor.

For example, the method may comprise administering to a subject in need: an effective amount of the aforementioned PI3K inhibitor, such as the aforementioned (S)-2-(1-(9H-purin-6-ylamino) propyl group)-3-(3-fluorophenyl)-4H-chromen-4-one or a pharmaceutically acceptable salt thereof and the aforementioned anti-PD-L1 antibody, for example, Atezolizumab, Avelumab, BMS-936559, MPDL3280A, durvalumab, or a combination of them.

For example, the method may comprise administering to a subject in need: an effective amount of the aforementioned PI3K inhibitor, such as the aforementioned (S)-2-(1-(9H-purin-6-ylamino) propyl group)-3-(3-fluorophenyl)-4H-chromen-4-one or a pharmaceutically acceptable salt thereof and the aforementioned TGFβ inhibitors, for example, Fresolimumab, SB525334 or a combination thereof.

For example, the method may comprise administering to a subject in need: an effective amount of the aforementioned PI3K inhibitor, such as the aforementioned (S)-2-(1-(9H-purin-6-ylamino) propyl group)-3-(3-fluorophenyl)-4H-chromen-4-one or a pharmaceutically acceptable salt thereof; the aforementioned TGFβ inhibitors, for example, Fresolimumab, SB525334 or their combination; the aforementioned anti-PD-L1 Antibodies, e.g., Atezolizumab, Avelumab, BMS-936559, MPDL3280A, durvalumab, or combinations thereof.

For example, the method may comprise administering to a subject in need: an effective amount of the aforementioned PI3K inhibitor, such as the aforementioned (S)-2-(1-(9H-purin-6-ylamino) propyl group)-3-(3-fluorophenyl)-4H-chromen-4-one or a pharmaceutically acceptable salt thereof; the aforementioned TGFβ inhibitors, for example, Fresolimumab, SB525334 or their combination; the aforementioned anti-PD-L1 Antibodies, e.g. Nivolumab, pembrolizumab, pidilizumab, or a combination thereof.

In certain embodiment, the method includes administering to a subject in need: an effective amount of the aforementioned PI3K inhibitor and the aforementioned PD-L1/TGFβ dual inhibitor.

For example, the method may comprise administering to a subject in need: an effective amount of the aforementioned PI3K inhibitor, such as the aforementioned (S)-2-(1-(9H-purin-6-ylamino) propyl group)-3-(3-fluorophenyl)-4H-chromen-4-one, and the aforementioned PD-L1/TGFβ dual inhibitors, such as WBP1126, M7824, SHR-1701, or variants or biosimilars thereof, or a combination thereof.

In certain embodiment, the PI3K inhibitor can be administered simultaneously with the PD-L1/TGFβ dual inhibitor.

In certain embodiment, the PI3K inhibitor can be administered after the PD-L1/TGFβ dual inhibitor.

In certain embodiment, the PI3K inhibitor can be administered before the PD-L1/TGFβ dual inhibitor.

In some embodiment, it also includes administering an effective amount of the aforementioned small molecule TGFβ inhibitor to a subject in need.

In certain embodiment, the tumor may include solid tumors and non-solid tumors.

In certain embodiment, the tumor may comprise a tumor with abnormal expression of PI3K.

In some embodiment, the tumor may include a tumor with abnormal expression of PD-L1.

In certain embodiment, the tumor may include a tumor with abnormal expression of TGFβ.

In certain embodiment, the tumor may include a tumor of the gastrointestinal tract, melanoma, or lymphoma.

In certain embodiment, the methods of the present application delay resistance compared to the time at which resistance would normally develop when a subject is treated with either agent or inhibitor alone as a monotherapy. In certain embodiment, resistance is delayed by at least 2 weeks, e.g., at least 2 weeks, 4 weeks, 1 month, 2 months, 3 months, 4 months, 5 months, 6 months, 8 months, 10 months, 12 months, 1 year, 2 years, 4 years, 6 years, 8 years or longer. In certain embodiment, according to the method, the remission (e.g., complete remission or partial remission) is prolonged compared to the time period that remission would typically last if the subject was treated with either agent or inhibitor alone as monotherapy. In certain embodiment, the remission (e.g., complete remission or partial remission) is prolonged by at least 2 weeks, e.g., at least 2 weeks, 4 weeks, 1 month, 2 months, 3 months, 4 months, 5 months, 6 months, 8 months, 10 months, 12 months, 1 year, 2 years, 4 years, 6 years, 8 years or longer.

In certain embodiment, administrating a PI3K inhibitor or a second agent to the treatment regimen increases or restores sensitivity to the agent that the cancer is resistant to. For example, in certain embodiment, adding a second agent to the treatment regimen increases or restores sensitivity to a cancer-resistant PI3K inhibitor.

In certain embodiment, wherein the remission of the cancer in the subject is prolonged.

In certain embodiment, wherein the subject experiences a complete remission of cancer.

In certain embodiment, wherein the level of minimal residual disease (MRD) is reduced.

In certain embodiment, the methods in the present application comprise selecting a subject for treatment with a combination of a PI3K inhibitor and a second agent. In certain embodiment, a subject (e.g., a patient with a cancer, such as a cancer described herein) is selected for treatment with the combination based on the MRD in the subject. In certain embodiment, selection is based on the presence of MRD above a preselected level (e.g., 1 malignant cell in 100 normal cells, 1 malignant cell in 1000 normal cells, or 1 in 10,000 normal cells with 1 malignant tumor cell). Methods for monitoring minimal residual disease negativity (MRD) are known in the art. See, e.g., Zhou, J. et al., Blood, 2007, 110:1607-1611. Such methods include DNA-based tests or RNA-based tests. In certain embodiment, MRD is monitored using flow cytometry, sequencing, or PCR.

In another aspect, the application provides the aforementioned pharmaceutical combination or the aforementioned pharmaceutical composition in the preparation of medicines for treating and/or preventing tumors.

For example, the use of the aforementioned PI3K inhibitors and the aforementioned immune checkpoint inhibitors in the preparation of drugs for treating and/or preventing tumors. For example, the use of the aforementioned PI3K inhibitor and the aforementioned TGFβ inhibitor in the preparation of medicaments for treating and/or preventing tumors. For example, the use of the aforementioned PI3K inhibitors, the aforementioned immune checkpoint inhibitors and the aforementioned TGFβ inhibitors in the preparation of drugs for treating and/or preventing tumors.

In another aspect, the present application provides the aforementioned pharmaceutical combination or the aforementioned pharmaceutical composition for treating and/or preventing tumors.

In another aspect, the present application provides a method for inhibiting tumor growth, the method comprising exposing the tumor to the aforementioned pharmaceutical combination or the aforementioned pharmaceutical composition.

In certain embodiment, the method comprises exposing the tumor to an aforementioned PI3K inhibitor and an aforementioned inhibitor of an immune checkpoint. In certain embodiment, the method comprises contacting the tumor with the aforementioned PI3K inhibitor and the aforementioned TGFβ inhibitor. In certain embodiment, the method comprises contacting the tumor with an aforementioned PI3K inhibitor, an aforementioned inhibitor of an immune checkpoint, and an aforementioned TGFβ inhibitor.

In another aspect, the present application provides a method for inhibiting tumor growth, the method comprising exposing the tumor to the aforementioned PI3K inhibitor and the aforementioned dual PD-L1/TGFβ inhibitor.

In another aspect, the present application provides a kit, which includes: (1) a first container, and the aforementioned PI3K inhibitor located in the first container; (2) a second container, and the aforementioned PD-L1/TGFβ dual inhibitors located in the second container.

In another aspect, the present application provides a kit, which may include: (1) a first container, and the aforementioned PI3K inhibitor located in the first container; (2) a second container, and the aforementioned immune checkpoint inhibitor or the aforementioned TGFβ inhibitor located in the second container.

In certain embodiment, the kit further includes (3) a third container, and the aforementioned immune checkpoint inhibitor or the aforementioned TGFβ inhibitor located in the third container, the agent in the third container different from the medicament in the second container. In certain embodiment, the dosage form of the medicine in the kit can be oral dosage form or injection dosage form

In another aspect, the present application provides a method for inhibiting tumor growth, said method comprising exposing the tumor to the aforementioned PI3K inhibitor and the aforementioned dual PD-L1/TGFβ inhibitor.

In another aspect, the present application provides a kit, which includes: (1) a first container, and the aforementioned PI3K inhibitor located in the first container; (2) a second container, and the aforementioned PD-L1/TGFβ dual inhibitors located in the second containers.

In another aspect, the present application provides a kit, which includes: (1) a first container, and the aforementioned PI3K inhibitor located in the first container; (2) a second container, and the aforementioned TGFβ inhibitors located in the second containers.

In certain embodiment, the kit further includes (3) a third container, and the aforementioned immune checkpoint inhibitor or the aforementioned TGFβ inhibitor located in the third container, the agent in the third container is different from the medicament in the second container.

In certain embodiment, the dosage form of the medicine in the kit can be oral dosage form or injection dosage form.

In certain embodiment, the kit can also contain instructions.

Without intending to be limited by any theory, the following examples are only to illustrate the pharmaceutical combination, preparation method and use of the present application, and not to limit the scope of the invention

### EXAMPLE

### Materials and Method

The animal experiment was carried out in accordance with the requirements of AAALAC, and was approved by Bikai Animal Experiment Center IACUC to conduct. 6-8 week-old Balb/c mice (16-20 grams) were purchased from Beijing Weitong Lihua Experimental Animal Technology Co., Ltd. Each mouse was subcutaneously inoculated with 500,000 A20 cells, and inoculated on the right rear side of the back of the mouse. When the volume of the tumor grew to about 60mm³, the mice were randomly assigned to the control group and the experimental group.

Mice were euthanized whnmorbidity or weightloss>20% was observed. Tumors were measured with a caliper twice weekly until final sacrifice. When a tumor size reached approximately 2000 mm³ or there were animal health issues (20% area of a tumor ulcerated), the animals would be euthanized an date of death recorded. Solid tumor volumes were estimated from two dimensional tumor measurements and calculated according to the following equation:

Tumor volume (mm 3)=[length(mm)×width2 (mm 2)]/2

The percent median regression for a group at a given day was then obtained by taking the median of the individual percent regression calculated for each animal of the group at this day. The day of calculation was determined on the day when ΔT/ΔC (i.e., the ratio of medians of tumor volume changes from baseline between the treated and control groups)was calculated, except when median percent regression was not representative of the activity of the group. In that case, the day was determined on the first day when the median percent regression was maximal. Regressions were defined as partial (PR) if the tumor volume decreased to 50% of the tumor volume at the start of treatment. Complete regression (CR) was considered to have been achieved when tumor volume was below 14 mm³ or could not be recorded.
RTV: Relative Tumor Volume. RTV=Vₜ/V_{0∘} Vₜ: Tumor volume at the end of an experimental period (treatment group). V₀: tumor volume at the beginning of the experiment
TGI: Tumor Growth Inhibition value. TGI=[1−RTV(experimental group)/RTV(control group)]*100%

### Statistical Analysis

A two-way ANOVA type with factors treatment an day(repeated) was performed on tumor volume changes from baseline. In case of significant treatment day interaction or treatment effects, it was followed by a contrast analysis with Bonferroni-Holm correction for multiplicity to compare all treated groups to the control group at each day from day 8 to 27. Tumor volume changes from baseline were calculated for each animal and each day by subtracting the tumor volume on the day of first treatment from the tumor volume on the specified observation day. As heterogeneity of variances was observed between groups, the compound symmetric(CS) covariance structure with group=option was chosen for the ANOVA type model (SAS Institute Inc. (2008) SAS/STAT 9.2User′s Guide by CaryNC). All statistical analyses were performed using SAS version v9.2 software. A probability less than 5% (p<0.05).

### Example 1 Study of the Combination of PI3K Inhibitors With TGFβ/PDL1 Bispecific Antibody With Different Dosage

### Example 1.1 Study on the Combination of CN401 (Tenalisib) and CN202 (WBP1126) (5 mg/kg) in the A20 Mouse B-Cell Lymphoma Model (See Table 2 for the Test Design)

6-8 weeks old balb/c mice (18-20 g) were purchased from Beijing Weitong Lihua Experimental Animal Technology Co., Ltd., and each mouse was subcutaneously inoculated with 500,000 A20 cells on the right rear side of the mouse's back. Treatment was initiated when tumors grew to a volume of approximately 62 mm³. CN401 was orally administered at 150 mg/kg twice a day for 3 weeks.

CN202 was administered by intraperitoneal injection, 3 times a week at a dose of 5 mg/kg for 3 weeks. The control group was treated with vehicle (0.5% MC and PBS) without active product. Tumor size and body weight were measured three times a week. This animal experiment was carried out in accordance with the requirements of AAALAC, and was approved by Bikai Animal Experiment Center IACUC to conduct this animal experiment.

TABLE 2

| Group | Amount | Method of administration |
|---|---|---|
| 1 | 8 | Vehicle control group (PBS i.p. tiw × 3 weeks + |
| | | 0.5% MC p.o. bid × 3 weeks) |
| 2 | 8 | CN401 150 mg/kg, p.o. bid × 3 weeks |
| 3 | 8 | CN202 5 mg/kg, i.p. tiw × 3 weeks |
| 4 | 8 | CN401 150 mg/kg, p.o. bid × 3 weeks + CN202 |
| | | 5 mg/kg, i.p. tiw × 3 weeks |

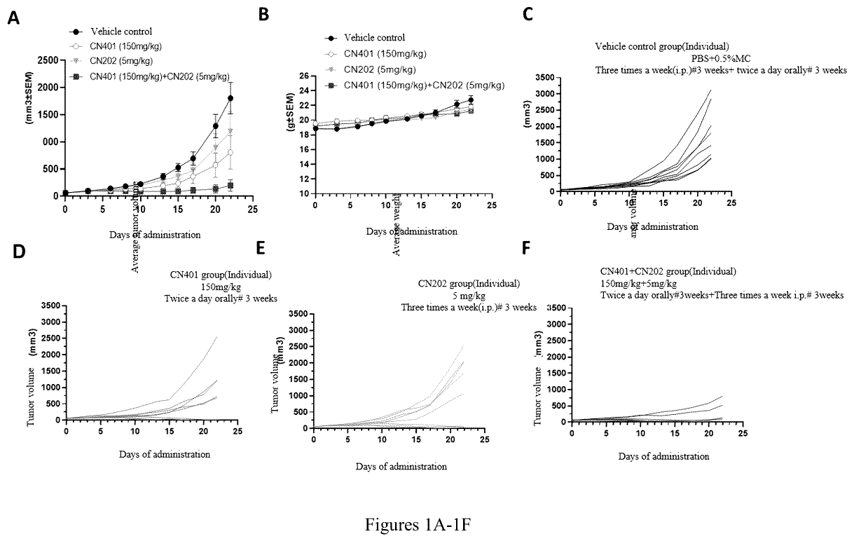

The results of tumor growth curves and body weight growth curves are shown in FIG. 1A-FIG. 1F; □GI, p-value and number of complete responding mice in the treatment group compared with the blank group at day 22 are shown in Table 3.

Result: In this study, the combination of CN401 and CN202 had a synergistic effect on tumor suppression in the A20 mouse B-cell lymphoma model (p=0.0001, TGI=89%, CR= 4/8). The mice showed good tolerance to the dosing regimen.

TABLE 3

| | | | CR (Number of |
|---|---|---|---|
| | | p value | mice with tumor |
| | | (vs vehicle | regression/total |
| | | control | amount of mice |
| Group# | TGI % | group) | in each group) |
| CN401 150 mg/kg | 55 | 0.034 | 2/8 |
| CN202 5 mg/kg | 31 | 0.27 | 1/8 |
| CN401 150 mg/kg + | 89 | 0.00010 | 4/8 |
| CN202 5 mg/kg | | | |

### Example 1.2 Study on the Combination of CN401 and CN202 (15 mg/kg) in the A20 Mouse B-Cell Lymphoma Model (See Table 4 for the Test Design)

Referring to the experimental method of Example 1.1, the difference is that the dosage of CN202 is replaced by 15 mg/kg from 5 mg/kg.

TABLE 4

| Group | Amount | Method of administration |
|---|---|---|
| 1 | 8 | Vehicle control group (PBS i.p. tiw × 3 weeks + |
| | | 0.5% MC p.o. bid × 3 weeks) |
| 2 | 8 | CN401 150 mg/kg, p.o. bid × 3 weeks |
| 3 | 8 | CN202 15 mg/kg, i.p. tiw × 3 weeks |
| 4 | 8 | CN401 150 mg/kg, p.o. bid × 3 weeks + CN202 |
| | | 15 mg/kg, i.p. tiw × 3 weeks |

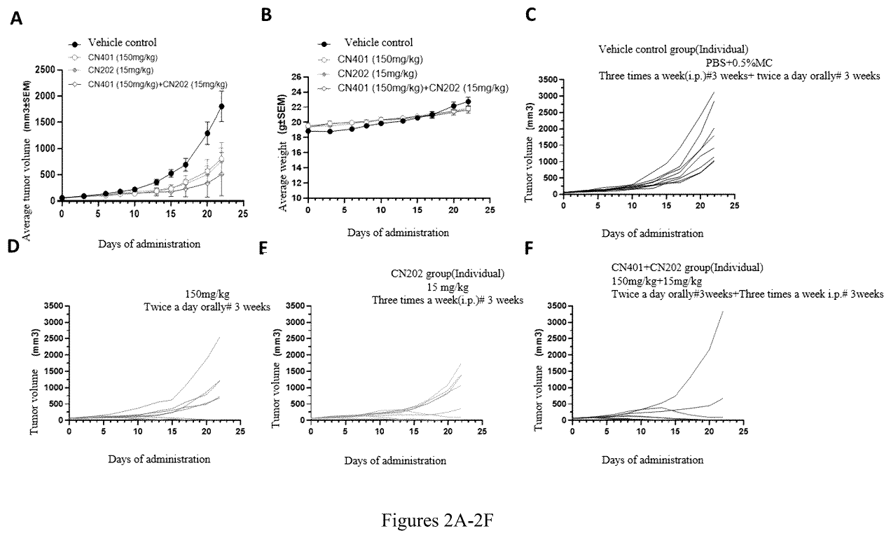

The results of tumor growth curves and body weight growth curves are shown in FIG. 2A-FIG. 2F; □GI, p-value and number of complete responding mice in the treatment group compared with the blank group at day 22 are shown in Table 5.

Result: In this study, the combination of CN401 and CN202 had a synergistic effect on tumor suppression in the A20 mouse B-cell lymphoma model (p=0.023, TGI=71%, CR=⅝). The mice showed good tolerance to the dosing regimen.

TABLE 5

| | | | CR (Number of |
|---|---|---|---|
| | | p value | mice with tumor |
| | | (vs vehicle | regression/total |
| | | control | amount of mice |
| Group# | TGI % | group) | in each group) |
| CN401 150 mg/kg | 55 | 0.034 | 2/8 |
| CN202 15 mg/kg | 58 | 0.014 | 2/8 |
| CN401 150mg/kg + | 71 | 0.023 | 5/8 |
| CN202 15 mg/kg | | | |

### Example 2 Study of the Combination of TGFβ/PDL1 Bispecific Antibody With Different Forms Of PI3K Inhibition

### Example 2.1 Study of the Combination of CN202(WBP1126) With CN401(Tenalisib) (see Table 6 for the Test Design)

6-8 weeks old balb/c mice (18-20 g) were purchased from Beijing Weitong Lihua Experimental Animal Technology Co., Ltd., and each mouse was subcutaneously inoculated with 500,000 A20 cells on the right rear side of the mouse's back. Treatment was initiated when tumors grew to a volume of approximately 80 mm³.

CN202 was administered by intraperitoneal injection, 3 times a week at a dose of 5 mg/kg for 3 weeks. CN401 was orally administered at 150 mg/kg twice a day for 3 weeks. The control group was treated with vehicle (0.5% MC and PBS) without active product. Tumor size and body weight were measured three times a week. This animal experiment was carried out in accordance with the requirements of AAALAC, and was approved by Bikai Animal Experiment Center IACUC to conduct this animal experiment.

TABLE 6

| Group | Amount | Method of administration |
|---|---|---|
| 1 | 7 | Vehicle control group (PBS i.p. tiw × 3 weeks + |
| | | 0.5% MC p.o. bid × 3 weeks) |
| 2 | 7 | CN202 5 mg/kg i.p. tiw × 3 weeks |
| 3 | 7 | CN401 150 mg/kg p.o. bid × 3 weeks |
| 4 | 7 | CN202 5 mg/kg i.p. tiw× 3 weeks + CN401 |
| | | 150 mg/kg p.o. bid × 3 weeks |

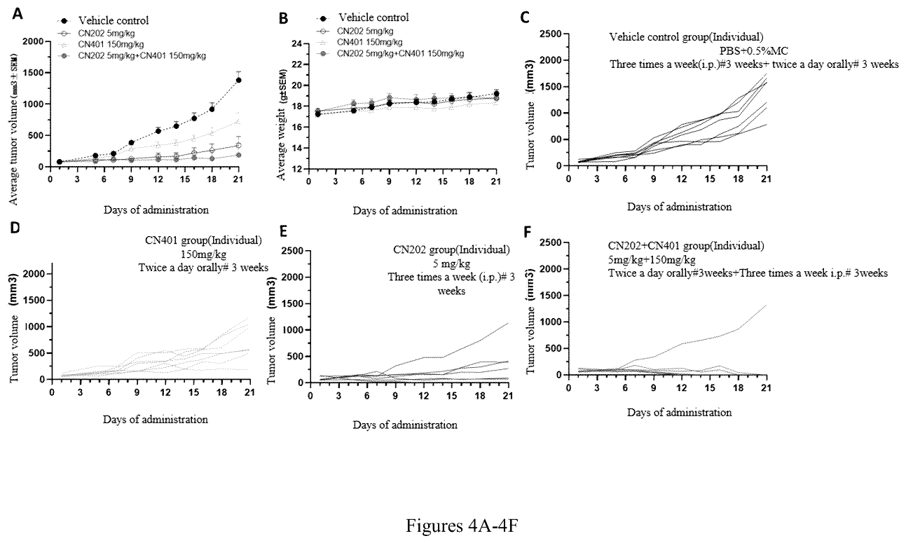

The results of tumor growth curves and body weight growth curves are shown in FIG. 4A-FIG. 4F; □GI, p-value and number of complete responding mice in the treatment group compared with the blank group at day 22 are shown in Table 7.

Result: In this study, the combination of CN202 and CN401 had a synergistic effect on tumor suppression in the A20 mouse B-cell lymphoma model (p=0.0009, TGI=85%, CR= 6/7). The mice showed good tolerance to the dosing regimen.

TABLE 7

| | | | CR(Number of |
|---|---|---|---|
| | | P value | mice with tumor |
| | | (vs vehicle | regression/total |
| | | control | amount of mice |
| Group | TGI % | group) | in each group) |
| CN202 5 mg/kg | 68 | 0.004 | 1/7 |
| CN401 150 mg/kg | 45 | 0.028 | 0/7 |
| CN202 5 mg/kg + | 85 | 0.0009 | 6/7 |
| CN401 150 mg/kg | | | |

### Example 2.2 Study of the Combination of CN202(WBP1126) With Alpelisib (See Table 8 for the Test Design)

Referring to the experimental method of Example 2.1, the difference is that the PI3K inhibitor was replaced by oral administration of CN401 (Tenalisib) at 150 mg/kg twice a day for 3 weeks to Alpelisib at 50 mg/kg once a day for 3 weeks.

TABLE 8

| Group | Amount | Plan for Administration |
|---|---|---|
| 1 | 7 | Vehicle control group (PBS i.p. tiw × 3 weeks + |
| | | 0.5% MC p.o. bid × 3 weeks) |
| 2 | 7 | CN202 5 mg/kg i.p. tiw × 3 weeks |
| 3 | 7 | Alpelisib 50 mg/kg p.o. qd × 3 weeks |
| 4 | 7 | CN202 5 mg/kg i.p. tiw × 3 weeks + Alpelisib |
| | | 50 mg/kg p.o. qd × 3 weeks |

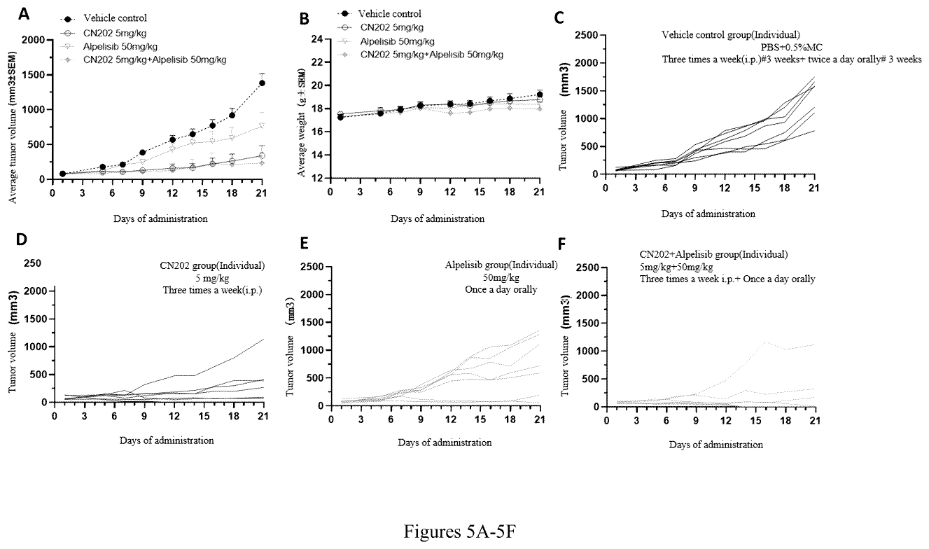

The results of tumor growth curves and body weight growth curves are shown in FIG. 5A-FIG. 5F; □GI, p-value and number of complete responding mice in the treatment group compared with the blank group at day 21 are shown in Table 9.

Result: In this study, the combination of CN202 and Alpelisib had a synergistic effect on tumor suppression in the A20 mouse B-cell lymphoma model (p=0.0002, TGI=85%, CR= 3/7). The mice showed good tolerance to the dosing regimen.

TABLE 9

| | | | CR(Number of |
|---|---|---|---|
| | | p value | mice with tumor |
| | | (vs vehicle | regression/total |
| | | control | amount of mice |
| Group# | TGI % | group) | in each group) |
| CN202 5 mg/kg | 68 | 0.004 | 1/7 |
| Alpelisib 50 mg/kg | 37 | 0.161 | 0/7 |
| CN202 5 mg/kg + | 85 | 0.0002 | 3/7 |
| Alpelisib 50 mg/kg | | | |

### Example 3 Study of the Combination of CN401 With Anti-PD-L1 Antibody in the A20 Mouse B-Cell Lymphoma Model (See Table 8 for the Test Design)

6-8 weeks old balb/c mice (18-20 g) were purchased from Beijing Weitong Lihua Experimental Animal Technology Co., Ltd., and each mouse was subcutaneously inoculated with 500,000 A20 cells on the right rear side of the mouse's back. Treatment was initiated when tumors grew to a volume of approximately 62 mm³.

CN401 was orally administered at 150 mg/kg twice a day for 3 weeks. Anti-PD-L1 antibody(Atezolizumab) was administered by intraperitoneal injection, 3 times a week at a dose of 5 mg/kg for 3 weeks. The control group was treated with vehicle (0.5% MC and PBS) without active product. Tumor size and body weight were measured three times a week. This animal experiment was carried out in accordance with the requirements of AAALAC, and was approved by Bikai Animal Experiment Center IACUC to conduct this animal experiment.

TABLE 10

| Group | Amount | Method of administration |
|---|---|---|
| 1 | 8 | Vehicle control group(PBS i.p. biw × 3 weeks + |
| | | 0.5% MC p.o. bid × 3 weeks) |
| 2 | 8 | CN401 150 mg/kg, p.o. bid × 3 weeks |
| 3 | 8 | Anti-PD-L1 Ab (atezolizumab) 5 mg/kg, i.p. biw × |
| | | 3 weeks |
| 4 | 8 | CN401 150 mg/kg, p.o. bid × 3 weeks + anti-PD-L1 |
| | | Ab (atezolizumab) 5 mg/kg, i.p. biw × 3 weeks |

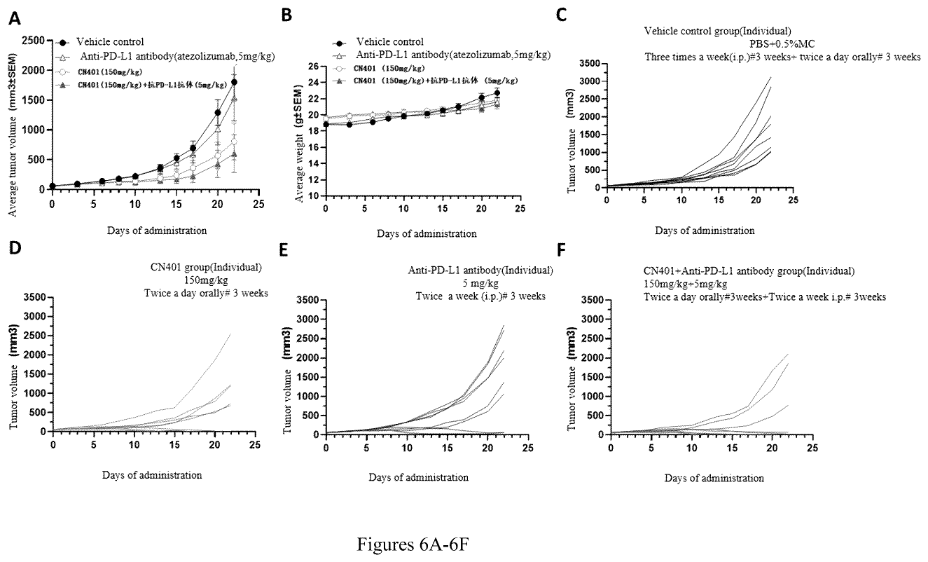

The results of tumor growth curves and body weight growth curves are shown in FIG. 6A-FIG. 6F; □GI, p-value and number of complete responding mice in the treatment group compared with the blank group at day 22 are shown in Table 11.

Result: In this study, the combination of CN401 and anti-PD-L1 antibody had a synergistic effect on tumor suppression in the A20 mouse B-cell lymphoma model (p=0.026, TGI=63%, CR=⅜). The mice showed good tolerance to the dosing regimen.

TABLE 11

| | | | CR(Number of |
|---|---|---|---|
| | | p value | mice with tumor |
| | | (vs vehicle | regression/total |
| | | control | amount of mice |
| Group# | TGI % | group) | in each group) |
| CN401 150 mg/kg | 55 | 0.034 | 2/8 |
| Anti-PD-L1 Ab 5 mg/kg | 15 | 0.57 | 0/8 |
| CN401 150 mg/kg + | 63 | 0.026 | 3/8 |
| anti-PD-L1 Ab 5 mg/kg | | | |

### Example 4 Study of Combination of CN202 and Nab-Pac and the Combination of CN401, CN202 and Nab-Pac in EMT-6 Tumor Model (See Table 12 for Experimental Design)

6-8 weeks old balb/c mice (18-20 g) were purchased from Beijing Weitong Lihua Experimental Animal Technology Co., Ltd., and each mouse was subcutaneously inoculated with 500,000 EMT-6 cells (mouse triple-negative breast cancer cells), inoculated in the 2-3 pairs of mammary gland fat pads on the left side of the mouse chest from top to bottom. CN401 was administered orally at 150 mg/kg twice daily for 4 weeks. CN202 and nab-paclitaxel were administered by intraperitoneal injection. CN202 was administered 3 times a week at a dose of 15 mg/kg for 2 weeks; with the same dose, CN202 was administered once a week for 2 weeks. Nab-paclitaxel was administered twice weekly at a dose of 10 mg/kg for 2 weeks. The control group was treated with vehicle (0.5% MC and PBS) without active product. Tumor size and body weight were measured three times a week. This animal experiment was carried out in accordance with the requirements of AAALAC, and was approved by Bikai Animal Experiment Center IACUC to conduct this animal experiment.

TABLE 12

| Group | Amount | Group | Plan of administration |
|---|---|---|---|
| 1 | 8 | Vehicle | Vehical (PBS i.p., three times a |
| | | control group | week × 2 week + 0.5% MC oral |
| | | | twice a day × 2 week) |
| 2 | 8 | CN202 | CN202 15 mg/kg, i.p., (three |
| | | 15 mg/kg | times a week × 2 week + once a |
| | | | week × 2 week) |
| 3 | 8 | CN401 | CN401 150 mg/kg, oral, twice a |
| | | 150 mg/kg | week × 4 week |
| 4 | 8 | Nab- | Nab-paclitaxel 10 mg/kg, i.p., |
| | | paclitaxel | twice a week × 2 week |
| | | 10 mg/kg | |
| 5 | 8 | CN202 | CN202 15 mg/kg, i.p., (three |
| | | 15 mg/kg + | times a week × 2 week + once a |
| | | CN401 | week × 2 week) + CN401 |
| | | 150 mg/kg | 150 mg/kg, oral, twice a week × |
| | | | 4 week |
| 6 | 8 | CN202 | CN202 15 mg/kg, i.p., (three |
| | | 15 mg/kg + | times a week × 2 week + once a |
| | | Nab- | week × 2 week) + Nab-paclitaxel |
| | | paclitaxel | 10 mg/kg, i.p., twice a week × |
| | | 10 mg/kg | 2 week |
| 7 | 8 | CN202 | CN202 15 mg/kg, i.p., (three |
| | | 15 mg/kg + | times a week × 2 week + once a |
| | | CN401 | week × 2 week) + CN401 |
| | | 150 mg/kg + | 150 mg/kg, oral, twice a day × |
| | | Nab- | 4 week + Nab-paclitaxel 10 mg/kg, |
| | | paclitaxel | i.p., twice a week × 2 week |
| | | 10 mg/kg | |

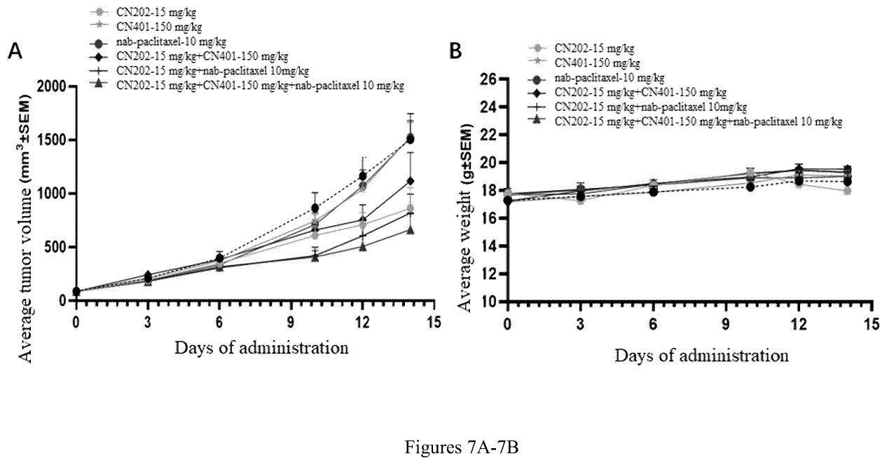

The results of tumor growth curve and body weight growth curve are shown in FIG. 7.

Results: This study found that in the orthotopic model of triple-negative breast cancer in EMT-6 mice, the combination of CN202 and nab-paclitaxel had a certain synergistic effect on tumor suppression (p=0.06, TGI=48% (at 14 days), CR=⅜ (at 80 days)); CN401, CN202 combined with nab-paclitaxel had a good synergistic effect on tumor suppression (p=0.02, TGI=56% (at 14 days), CR= 4/8 (within 80 days)). The dosing regimen was well tolerated by mice. The results of the tumor growth curve and body weight growth curve are shown in FIG. 7A. According to the IACUC regulations of Bikai Animal Experiment Center, the individual tumor volume≥2000 mm³ needs to be euthanized, and the results are shown in FIG. 7B; comparing to the vehicle control group on the 14^{th} day, p-values and the number of complete responding mice (observed up to 80 days) are shown in Table 13.

TABLE 13

| | | | CR(Number of |
|---|---|---|---|
| | | p value | mice with tumor |
| | | (vs vehicle | regression/total |
| | | control | amount of mice |
| Group | TGI % | group) | in each group) |
| CN202 15 mg/kg | 42 | 0.11 | 0/8 |
| CN401 150 mg/kg | 3 | 0.87 | 0/8 |
| Nab-paclitaxel 10 mg/kg | 2 | 0.9 | 0/8 |
| CN202 15 mg/kg + | 28 | 0.3 | 1/8 |
| CN401 150 mg/kg | | | |
| CN202 15 mg/kg + | 48 | 0.06 | 3/8 |
| Nab-paclitaxel 10 mg/kg | | | |
| CN202 15 mg/kg + | 56 | 0.02 | 4/8 |
| CN401 150 mg/kg + | | | |
| Nab-paclitaxel 10 mg/kg | | | |

1-105. (canceled)

106. A pharmaceutical combination, comprising a phosphoinositide 3-kinase (PI3K) inhibitor and a second therapeutic agent, wherein the second therapeutic agent is a PD-L1/TGFβ dual inhibitor.

107. The pharmaceutical combination according to claim 106, wherein the PD-L1/TGFβ dual inhibitor comprises an anti-PD-L1 antibody or an antigen-binding fragment thereof and a TGFβ receptor domain.

108. The pharmaceutical combination according to claim 107, wherein the anti-PD-L1 antibody or antigen-binding fragment thereof comprises a heavy chain variable region of an antibody, wherein the heavy chain variable region comprises HCDR1, HCDR2, HCDR3, the said HCDR1 comprises the amino acid sequence shown in SEQ ID NO: 1 or an amino acid sequence obtained by amino acid addition, elimination or substitution reaction with no more than 2 amino acid differences from the amino acid sequence shown in SEQ ID NO: 1; HCDR2 comprises the amino acid sequence shown in SEQ ID NO: 2 or an amino acid sequence obtained by amino acid addition, elimination or substitution reaction with no more than 2 amino acid differences from the amino acid sequence shown in SEQ ID NO: 2; the HCDR3 comprising the amino acid sequence shown in SEQ ID NO: 3 or an amino acid sequence obtained by amino acid addition, elimination or substitution reaction and having no more than 2 amino acid differences from the amino acid sequence shown in SEQ ID NO: 3.

109. The pharmaceutical combination according to claim 108, wherein the anti-PD-L1 antibody or antigen-binding fragment thereof comprises an antibody heavy chain variable region, wherein the heavy chain variable region comprises an amino acid sequence selected from the group consisting of: (a) the amino acid sequence set forth in SEQ ID NO: 4; (b) an amino acid sequence at least about 85%, 90%, 95% or 99% sequence identity to the amino acid sequence set forth in SEQ ID NO: 4; and (c) an amino acid sequence having 1 or more differences from the amino acid sequence shown in SEQ ID NO: 4 obtained by addition, elimination or substitution reaction.

110. The pharmaceutical combination of claim 107, wherein the PD-L1/TGFβ dual inhibitor comprises a polypeptide, wherein the polypeptide comprises at least: (i) a heavy chain variable region of the anti-PD-L1 antibody; and (ii) TGFβRII or a functionally active fragment thereof.

111. The pharmaceutical combination of claim 110, wherein the TGFβRII or a functionally active fragment thereof comprises:
(a) the amino acid sequence of SEQ ID NO: 6;
(b) an amino acid sequence having at least about 85% sequence identity to the amino acid sequence set forth in SEQ ID NO: 6; or
(c) an amino acid sequence in which one or more amino acids are added, deleted and/or substituted as compared to the amino acid sequence shown in SEQ ID NO: 6.

112. The pharmaceutical combination of claim 110, wherein the polypeptide further comprises a linker that links the C-terminus of the heavy chain variable region of the anti-PD-L1 antibody or antigen-binding fragment thereof to the N-terminus of the TGFβRII or functionally active fragment thereof.

113. The pharmaceutical combination according to claim 110, said polypeptide further comprising CH2, CH3 domains, said polypeptide comprising the heavy chain variable region (VH) of anti-PD-L1 antibody, CH2, CH3 domains and TGFβRII or a functionally active fragment thereof in sequence from N-terminus to C-terminus, said CH3 domain having its C-terminus connected to the N-terminus of said TGFβRII or a functionally active fragment thereof by a linker.

114. The pharmaceutical combination of claim 113, wherein the linker is a peptide linker.

115. The pharmaceutical combination of claim 114, wherein the amino acid sequence of the peptide linker is (G₄S)ₓ, wherein x is any integer from 3 to 6.

116. The pharmaceutical combination of claim 114, wherein the peptide linker comprises an amino acid sequence selected from the group consisting of: (a) the amino acid sequence set forth in SEQ ID NO: 5; (b) an amino acid sequence having at least about 85%, 90%, 95%, or 99% sequence identity to the amino acid sequence set forth in SEQ ID NO: 5; and (c) an amino acid sequence having 1 or more differences from the amino acid sequence shown in SEQ ID NO: 5 obtained by addition, elimination or substitution reaction.

117. The pharmaceutical combination of claim 110, wherein the polypeptide comprises an amino acid sequence selected from the group consisting of: (a) the amino acid sequence set forth in SEQ ID NO: 7; (b) an amino acid sequence having at least about 85%, 90%, 95%, or 99% sequence identity to the amino acid sequence set forth in SEQ ID NO: 7; (c) an amino acid sequence having 1 or more differences from the amino acid sequence shown in SEQ ID NO: 7 obtained by addition, elimination or substitution reactions.

118. The pharmaceutical combination according to claim 106, wherein the PI3K inhibitor is a dual PI3K δ/γ inhibitor.

119. The pharmaceutical combination according to claim 106, wherein said PI3K inhibitor comprise: (S)-2-(1-(9H-purin-6-ylamino) propyl group)-3-(3-fluorophenyl)-4H-chromen-4-one or its pharmaceutically acceptable salts, solvates, hydrates or prodrugs.

120. The pharmaceutical composition according to claim 106, said pharmaceutical combination further comprises a third active substance, wherein the third active substance is paclitaxel (such as albumin bound paclitaxel).


SEQUENCE LISTING (7 sequences)

SEQ ID NO: 1 - protein, 9 aa, Artificial Sequence
  HCDR1
GHFSNLAVN

SEQ ID NO: 2 - protein, 17 aa, Artificial Sequence
  HCDR2
GILWSGGSTFYADSVKG

SEQ ID NO: 3 - protein, 3 aa, Artificial Sequence
  HCDR3
GTN

SEQ ID NO: 4 - protein, 111 aa, Artificial Sequence
  VHH
EVQLVESGGGLVQPGGSLRLSCAASGHFSNLAVNWFRQAPGKERELVAGILWSGGSTFYADSVKGRFTISRGNAENMLYLQMNSLRAEDTAVYYCNTGTNWGQGTLVTVSS

SEQ ID NO: 5 - protein, 20 aa, Artificial Sequence
  peptide linker
GGGGSGGGGSGGGGSGGGGS

SEQ ID NO: 6 - protein, 136 aa, Artificial Sequence
  TGFRII extracellular domain
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 7 - protein, 497 aa, Artificial Sequence
  polypeptide
EVQLVESGGGLVQPGGSLRLSCAASGHFSNLAVNWFRQAPGKERELVAGILWSGGSTFYADSVKGRFTISRGNAENMLYLQMNSLRAEDTAVYYCNTGTNWGQGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD